SEQUENCE LISTING (4 sequences)

SEQ ID NO: 1 - DNA, 39 bp, Artificial Sequence
  Primer for cDNA of cysteinyl proteinase (Falcipain 2)
cgcggatccgccaccatggaattaaacagatttgccgat

SEQ ID NO: 2 - DNA, 57 bp, Artificial Sequence
  Primer for cDNA of cysteinyl proteinase (Falcipain 2)
cgcgtcgacttaatgatgatgatgatgatgttcaattaatggaatgaatgcatcagt

SEQ ID NO: 3 - DNA, 886 bp, Artificial Sequence
  PCR product from amplification using primers for the cDNA sequence of cysteinyl proteinase (Falcipain 2)
ccatggaattaaacagatttgccgatttaacttatcatgaatttaaaaacaaatatcttagtttaagatcttcaaaaccattaaagaattctaaatatttattagatcaaatgaattatgaagaagttataaaaaaatatagaggagaagaaaatttcgatcatgcagcttacgactggagattacacagtggtgtaacacctgtaaaggatcaaaaaaattgtggatcttgctgggcctttagtagtataggttccgtagaatcacaatatgctatcagaaaaaataaattaataaccttaagtgaacaagaattagtagattgttcatttaaaaattatggttgtaatggaggtctcattaataatgcctttgaggatatgattgaacttggaggtatatgtccagatggtgattatccatatgtgagtgatgctccaaatttatgtaacatagatagatgtactgaaaaatatggaatcaaaaattatttatccgtaccagataataaattaaaagaagcacttagattcttgggacctattagtattagtgtagccgtatcagatgattttgctttttacaaagaaggtattttcgatggagaatgtggtgatgaattaaatcatgccgttatgcttgtaggttttggtatgaaagaaattgttaatccattaaccaagaaaggagaaaaacattattattatataattaagaactcatggggacaacaatggggagaaagaggtttcataaatattgaaacagatgaatcaggattaatgagaaaatgtggattaggtactgatgcattcattccattaattgaacatcatcatcatcatcattaagtcgacgcgatcgaattcctgcagcccggggatcc

SEQ ID NO: 4 - protein, 282 aa, Artificial Sequence
  PCR product from amplification using primers for the cDNA sequence of cysteinyl proteinase (Falcipain 2)
MELNRFADLTYHEFKNKYLSLRSSKPLKNSKYLLDQMNYEEVIKKYRGEENFDHAAYDWRLHSGVTPVKDQKNCGSCWAFSSIGSVESQYAIRKNKLITLSEQELVDCSFKNYGCNGGLINNAFEDMIELGGICPDGDYPYVSDAPNLCNIDRCTEKYGIKNYLSVPDNKLKEALRFLGPISISVAVSDDFAFYKEGIFDGECGDELNHAVMLVGFGMKEIVNPLTKKGEKHYYYIIKNSWGQQWGERGFINIETDESGLMRKCGLGTDAFIPLIEHHHHHH

(19) United States
(12) Patent Application Publication
(10) Pub. No.: US 2004/0229915 A1
(43) Pub. Date: Nov. 18, 2004

(54) Cysteine protease inhibitors

(75) Inventors: Martin Quibell, Cambridge (GB); Steven Taylor, Cambridge (GB); Urszula Grabowska, Cambridge (GB); Magnus Nilsson, Cambridge (GB); Veronique Morrison, Cambridge (GB)

Correspondence Address: BIRCH STEWART KOLASCH & BIRCH, PO BOX 747, FALLS CHURCH, VA, 22040-0747, US

(73) Assignee: MEDIVIR AB, Huddinge (SE)

(21) Appl. No.: 10/853,408

(22) Filed: May 24, 2004

Related U.S. Application Data
(63) Continuation of application No. 10/042,565, filed on Nov. 16, 2001.
Application No. 10/042,565 is a continuation-in-part of application No. 10/015,186, filed on Nov. 16, 2001.
Application No. 10/015,186 is a continuation-in-part of application No. PCT/GB00/01894, filed on May 18, 2000.
(60) Provisional application No. 60/252,802, filed on Nov. 17, 2000.
(60) Provisional application No. 60/252,840, filed on Nov. 17, 2000.

(30) Foreign Application Priority Data
May 18, 1999 (GB) 9911417.5

(51) Int. Cl. A61K 31/452; A61K 31/4025; A61K 31/366; C 07D 4 5/02

(52) U.S. Cl. 514/326; 514/336; 514/422; 514/460; 546/207; 546/282.1; 548/517; 549/293

(57) ABSTRACT

of the formula (IV):

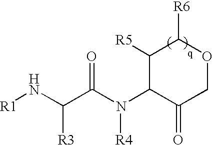

where:

R1=R′C(O), R′SO2,

R′=a bicyclic, saturated or unsaturated, 8-12 membered ring system containing 0-4 hetero atoms selected from S, O and N, which is optionally substituted with up to four substituents independently selected from groups a), b) and c) below; or

R′=a monocyclic, saturated or unsaturated, 5-7 membered ring containing 0-3 hetero atoms selected from S, O and N, which monocyclic ring bears at least one substituent selected from group a) and/or c) and which may optionally bear one or two further substituents selected from group b);

R4=H, C1-7-alkyl, Ar—C1-7-alkyl, Ar, C3-7-cycloalkyl; C2-7alkenyl,;

R3=C1-7-alkyl, C2-C7 alkenyl, C2-C7 alkenyl, C3-7-cycloalkyl, Ar—C1-7-alkyl, Ar;

R5=C1-7-alkyl, halogen,Ar—C1-7-alkyl, C0-3-alkyl-CONR3R4 or a bulky amine

R6 is H, C1-7-alkyl, Ar—C1-7-alkyl, C1-3-alkyl-SO2-R^{ix}, C1-3-alkyl-C(O)—NHR^{ix} or CH₂XAr

q is 0 or 1

have utility as inhibitors of cysteine proteases such as cathepsin K and falcipain.

This application is a Continuation of co-pending application Ser. No. 10/042,565, filed on Nov. 16, 2001, which is a Continuation-In-Part of U.S. application Ser. No. 10/015,186 filed on Nov. 16, 2001, which is a Continuation-In-Part of PCT International Application NO. PCT/GB00/01894 filed on May 18, 2000, which was published in English and which designated the United States, which priority is claimed under 35 U.S.C. § 120, the entire contents of which are hereby incorporated by reference. This Continuation Application claims priority under 35 U.S.C. § 119(e) on U.S. Provisional Application Nos. 60/252,802 and 60/252,840, both filed on Nov. 17, 2000, the entire contents of which are hereby incorporated by reference.

### FIELD OF THE INVENTION

This invention relates to inhibitors of cysteine proteases, especially those of the papain superfamily. The invention provides novel compounds useful in the prophylaxis or treatment of disorders stemming from misbalance of physiological proteases such as cathepsin K, or pathogenic proteases such as malarial falcipain.

### DESCRIPTION OF THE RELATED ART

The papain superfamily of cysteine proteases is widely distributed in diverse species including mammals, invertebrates, protozoa, plants and bacteria. A number of mammalian cathepsin enzymes, including cathepsins B, F, H, K, L, N and S, have been ascribed to this superfamily, and inappropriate regulation of their activity has been implicated in a number of metabolic disorders including arthritis, muscular dystrophy, inflammation, glomerulonephritis and tumour invasion. Pathogenic cathepsin like enzymes include the bacterial gingipains, the malarial falcipains I, II, III et seq and cysteine proteases from Pneumocystis carinii, Trypanosoma cruzei and brucei, Crithidia fusiculata, Schistosoma spp.

The inappropriate regulation of cathepsin K has been implicated in a number of disorders including osteoporosis, gingival diseases such as gingivitis and periodontitis, Paget's disease, hypercalcaemia of malignancy and metabolic bone disease. In view of its elevated levels in chondroclasts of osteoarthritic synovium, cathepsin K is implicated in diseases characterised by excessive cartilege or matrix degradation, such as osteoarthritis and rheumatoid arthritis. Metastatic neoplastic cells typically express high levels of proteolytic enzymes that degrade the surrounding matrix and inhibition of cathepsin K may thus assist in treating neoplasias.

WO 98/50533 describes the use of compounds according to the formula (I).

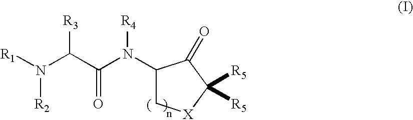

It is suggested the compounds of this formula, are useful as inhibitors to proteases, in particular the papain superfamily; specifically those of the Cathepsin family; and particularly Cathepsin K. The ketone bearing ring structure in these compounds has a tendency to spontaneously racemise, limiting their clinical utility. Other SKB applications describing ketone cathepsin K inhibitors include WO 98 46582, WO9964399, WO0029408, WO0038687 and WO0049011. However, none of these applications disclose α-ring substituents adjacent the linkage to the peptidomimetic chain.

Shenai et al, J Biol. Chem. 275 37 29000-29010 describes the isolation of a major cysteine protease, denoted falcipain 2 from trophozoites of Plasmodium falciparium. The enzyme appears inter alia to hydrolyse erythrocyte haemoglobin in acidic food vacuoles. This publication also describes the isolation of the corresponding gene using an N-terminus tag, which is autocatalytically removed during folding.

SmithKline Beecham's WO 99/53039 describes the cysteine protease inhibitory activity of a diverse range of peptidomimetics on a trophozoite preparation from Plasmodium falciparium. No guidance is provided as to which cysteine protease in being inhibited. Although most of the peptidomimetics are linear structures, one compound (R,S)-3-[N-(3-benzyloxybenzoyl)-L-leucinylamino]tetrahydrofuran-4-one belongs to the furanones of formula I depicted above. As would be expected of such structures, the ketone bearing ring is racemic.

Our copending PCT application WO00/69855 published after the present priority date, discloses cathepsin S inhibitors comprising a monocyclic P3 filling group.

### SUMMARY OF THE INVENTION

A first aspect of the invention provides compounds of the formula (IV):

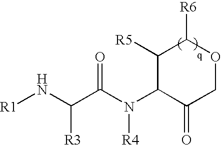

where:

R1=R′C(O)R′SO2,

R′=a bicyclic, saturated or unsaturated, 8-12 membered ring system containing 0-4 hetero atoms selected from S, O and N, which ring system is optionally substituted with up to four substituents independently selected from groups a), b) and c) below; or

R′=a monocyclic, saturated or unsaturated, 5-7 membered ring containing 0-3 hetero atoms selected from S, O and N, which monocyclic ring bears at least one substituent selected from group a) and/or c), and which may optionally bear one or two further substituents selected from group b);

a) a cyclic group which may be linked direct to the R′ ring or via an alkyl, alkylether, alkylthioether, alkylamine, alkylamide, alkylsulphonamide, alkylsulphone, alkylurea, alkylketone or alkylester linker; or

b) H, C1-7alkyl, C3-6cycloalkyl, OH, SH, NH₂, NHC1-3alkyl, N(C1-3alkyl)₂, halogen; or

c) O—C1-4alkyl, S—C1-4alkyl, SOC1-4alkyl, SO₂C1-4alkyl, CO2C0-4alkyl, NHCOC0-4alkyl, CONHC0-4alkyl, COC0-4alkyl, NHC(═NH)NH2;

R4=H, C1-7-alkyl, Ar—C1-7 alkyl, Ar, C3-7-cycloalkyl; C2-7alkenyl;

R3=C1-7-alkyl, C2-C7 alkenyl, C2-C7 alkenyl, C3-7-cycloalkyl, Ar—C1-7-alkyl, Ar;

R5=C1-7-alkyl, halogen, Ar—C1-7-alkyl, C0-3-alkyl-CONR3R4 or R^{iv};

R^{iv}=

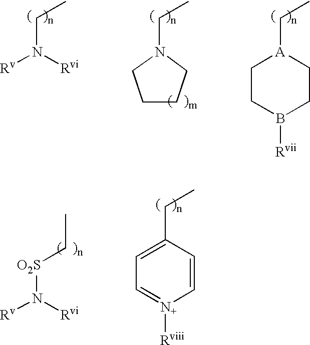

where n=1-3, m=1-3;

R^{v}, R^{vi}═H, C1-7-alkyl;

A=N, CH; B═N, O, S, CH;

R^{vii}=absent when B═O, S; or R^{vii}═H, C1-7-alkyl when B═N, CH;

R^{viii}═O, C1-7-alkyl;

R6=H, C1-7-alkyl, Ar—C1-7-alkyl, C1-3-alkyl-SO2-R^{ix}, C1-3-alkyl-C(O)—NHR^{ix} or CH₂XAr;

R^{ix} is C1-7-alkyl, C3-C6-cycloalkyl or Ar—C1-7-alkyl;

q is zero (ie —C(R6)- is a bond) or 1

and pharmaceutically acceptable salts thereof.

Compounds of the invention have utility in the treatment or prophylaxis of various disorders characterised by the presence or inappropriate activity of cysteine proteases of the papain superfamily, such as cathepsins B, F, L, S and especially cathepsin K or falcipain.

‘C1-7-alkyl’ as applied herein is meant to include straight and branched chain aliphatic carbon chains such as methyl, ethyl, n-propyl, isopropyl, n-butyl, isobutyl, t-butyl, pentyl, isopentyl, hexyl, heptyl and any simple isomers thereof. Additionally, any C1-7-alkyl may optionally be substituted by one or two halogens and/or a heteroatom S, O, NH. If the heteroatom is located at a chain terminus then it is appropriately substituted with one or 2 hydrogen atoms, for example hydroxymethyl. Sulphur heteroatoms may further be oxidised to sulphones.

‘C1-3-alkyl’ as applied herein includes methyl, ethyl, propyl, isopropyl, cyclopropyl, any of which may be optionally substituted as described in the paragraph above.

‘Amine’ includes NH2, NHC1-3-alkyl or N(C1-3-alkyl)2.

‘Halogen’ as applied herein is meant to include F, Cl, Br, I, particularly chloro and preferably fluoro.

‘C3-6-cycloalkyl’ (or C3-7-cycloalkyl) as applied herein is meant to include any variation of ‘C1-7-alkyl’ which additionally contains a C3-6 (or C3-7) carbocyclic ring such as cyclopropyl, cyclobutyl, cyclopentyl, cyclohexyl. Alternatively the C3-6 or C3-7 cycloalkyl may be spiro bound to the adjacent carbon without an intervening C1-C7 alkyl.

‘Ar—C1-7-alkyl’ as applied herein is meant to include a phenyl, pyrazolyl, pyridyl, imidazolyl, oxazolyl, isoxazolyl, thiazinolyl, isothiazinolyl, thiazolyl, oxadiazolyl, 1,2,3-triazolyl, 1,2,4-triazolyl, furanyl or thienyl aromatic ring (Ar) attached through a ‘C1-7-alkyl’ (defined above) to the dihydro-(3H)-furanone ring system or in the case of R2, R3 or R4 linked directly to the molecule backbone. Optionally, the aromatic ring Ar may be substituted with halogen, C1-3-alkyl, OH, OC1-3-alkyl, SH, SC1-3-alkyl, amine and the like.

The cyclic substituent a) to R′ may be saturated, unsaturated or aromatic and have 0 to 4 hetero atoms including monocyclic rings such as phenyl, cycloalkenyl, such as cyclohexenyl or cyclopentenyl, furyl, thienyl, pyranyl, pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, pyrazinyl, piperazinyl, pyrimidinyl, pyridazinyl, oxazolyl, oxazolidinyl, isoxazolyl, isoxazolidinyl, morpholinyl, thiazolyl, thiazolidinyl, isothiazolyl, isothiazolidinyl, and the like or bicyclic rings such as napthyl and especially any of the above fused to a phenyl ring such as indolyl, quinolinyl, isoquinolinyl, benzimidazolyl, benzothiazolyl, benzoxazolyl, benzothienyl etc. The carbo or heterocyclic ring substituent may be bonded via a carbon or via a hetero atom, typically a nitrogen atom, such as N-piperidyl, N-morpholinyl etc. The ring substituent a) may itself be substituted with substituents as for Ar above. The ring substituent a) excludes cycloalkyl as defined in subgroup b).

The optional alkyl, alkylether, alkylthioether, alkylamine, alkylamide, alkylsulphonamide, alkylsulphone, alkylurea, alkyketone or alkylester linkage between R′ and ring substituent a) may comprise up to 6 carbon atoms, typically up to four carbon atoms, for instance 1 or 2. If present, the ether, thioether, amine, amide, sulphonamide, sulphone, urea, ketone or ester component may be located adjacent the R′ ring, (for example a morpholinoethoxy substituent) adjacent the substituent ring (for example a phenylsulphonethyl substituent) or intermediate two alkyl groups, (for example a benzoyloxymethyl substituent).

‘C1-3-alkyl-CONR″’, R^{iv}′ as applied herein is meant to include straight or branched carbon chain substituted with a 1°, 2° or 3° carboxamide wherein R′″, R^{iv} includes H and Me.

‘C1-3-alkyl-SO₂—R^{ix}, as applied herein is meant to include straight or branched carbon chain substituted with a sulphone wherein R^{ix} includes ‘C1-7-alkyl’, ‘Ar—C1-7-alkyl’, ‘C3-6-cycloalkyl’.

‘C1-3-alkyl-C(O)—NHR^{ix}, as applied herein is meant to include straight or branched carbon chain substituted with a secondary carboxamide wherein R^{ix} includes ‘C1-7-alkyl’, ‘Ar—C1-7-alkyl’, ‘C3-6-cycloalkyl’.

Preferred R′ groups include bicyclic rings such as napthyl, quinoloyl, benzofuranyl, benzothienyl, indolyl and indolinyl, particularly where the linkage is to the 2 position of the R′ ring.

Additional bicyclic groups include naphthalenyl, especially naphthylen-2-yl; benzo[1,3]dioxolyl, especially benzo[1,3]dioxol-5-yl, benzofuranyl, especially benzofuran-2-yl, and especially C1-6 alkoxy substituted benzofuranyl, more especially 5-(2-piperazin-4-carboxylic acid tert-butyl ester-ethoxy)benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl; 7-methoxy-benzofuran-2-yl, 5-methoxy-benzofuran-2-yl, 5,6-dimethoxy-benzofuran-2-yl, especially halogen substituted benzofuranyl, more especially 5-fluoro-benzofuran-2-yl, 5,6-difluoro-benzofuran-2-yl, especially C 1-6alkyl substituted benzofuranyl, most especially 3-methyl-benzofuran-2-yl; benzo[b]thiophenyl, especially benzo[b]thiophen-2-yl; especially C1-6alkoxy substituted benzo[b]thiopheny], more especially 5,6-dimethoxy-benzo[b]thiophen-2-yl quinolinyl, especially quinolin-2-yl, quinolin-3-yl, quinolin-4-yl, quinolin-6-yl, and quinolin-S-yl; quinoxalinyl, especially quinoxalin-2-yl; 1,8 naphthyridinyl, especially 1,8 naphthyridin-2-yl; indolyl, especially indol-2-yl, especially indol-6-yl, indol-5-yl, especially C1-6alkyl substituted indolyl, more especially N-methylindol-2-yl; furo[3,2-b]pyridinyl, especially furo[3,2-b]pyridin-2-yl, and C1-6alkyl substituted furo[3,2-b]pyridinyl, especially 3-methyl-furo[3,2-b]pyridin-2-yl; thieno[3,2-b]thiophene, especially thieno[3,2-b]thiophene-2-yl, more especially Cl 6alkyl substituted thieno[3,2-b]thiophene-2-yl, more especially 5-tert-buty]-3-methylthieno[3,2-b]thiophene-2-yl.

Monocyclic R′groups include substituted pyridyl, substitute pyrimidyl, substituted phenyl, particularly phenyl substituted with a cyclic group such as pyrrolidine-1-yl, piperidine-1-yl, morpholin-4-yl, 4-methylpiperazin-1-yl, 2-morpholin-4-yl-ethylamino, and piperazin-1-yl. A phenyl R′ is conveniently substituted at the 3 or 4 position with such a cyclic group.

If a chiral centre is present, all isomeric forms are intended to be covered. Both (R) and (S) stereochemistries at the position corresponding to the furan 5-position (ie R5 adjacent the linkage to the peptidomimetic chain) are encompassed by the invention with (R) being convenient in some cases, for instance in cathepsin K or falcipain inhibitors, particularly in conjunction with R stereochemistry at the pyranone 4 bond. Alternatively R5 and the furanone/pyranone C4-bond conveniently both have the S stereochemistry.

The compounds of the invention are cysteine protease inhibitors, notably against cathepsins or cathepsin-like proteases of the papain superfamily. Ideally the compound displays selective inhibition of a single protease in the complex mixture of proteolytic enzymes characterising the physiological environment, for example a greater than 10-fold selectivity, preferably greater than 100 fold. Most preferably inhibitory specificity is exhibited over other members of the same enzyme class or family, such as the Cathepsin family, which have a high degree of homology, as incorrect regulation of proteolytic activity can lead to unwanted pathological conditions such as hypertension, blood clotting or worse. This is especially desirable for disorders such as autoimmune disorders where administration of the drug is likely to be protracted.

However, compounds can be useful notwithstanding that they exhibit a degree of promiscuity in relation to inhibition of physiological proteases. For example the physiological functions of many cathepsins are redundant, that is inhibition of a particular cysteine protease can be compensated by the presence or upregulation of other non-inhibited proteases or alternative metabolic routes.

Alternatively, treatments of short duration can result only in transient toxicity or other side effects.

The cross-specificity of cysteine proteases for a given putative inhibitor (ie the selectivity of the inhibitor) is readily ascertained with conventional enzyme and cell culture assays performed in parallel with the respective enzymes.

A further aspect of the invention comprises a method employing the compounds of formula IV for the treatment of diseases wherein cathepsin K is a factor, ie diseases or conditions alleviated or modified by inhibition of cathepsin K, preferably without substantial concomitant inhibition of other human members of the papain superfamily.

The invention further provides the use of the compounds of formula IV in therapy and in the manufacture of a medicament for the treatment of diseases or conditions alleviated or moderated by inhibition of cathepsin K

A further aspect of the invention provides methods for the treatment or prophylaxis of a parasitical infection such as a protozoal or bacterial infection comprising the administration of a compound of formula IV, to a mammal in need thereof. A still further aspect provides a method for the control of protozoal parasites comprising the administration of a compound of formula IV but without the proviso, to an invertebrate vector and/or to a locus prone to infestation of such a vector.

Conveniently the protozoal or bacterial parasite is a Plasmodium, Leishmania, Schistosoma, Giardia, Entamoeba, Trypansoma, Crithidia, Pneumocystis or Porphyromonas species.

Suitably, the treatment or prophylaxis of Plasmodium falciparium comprises inhibition of a falcipain II enzyme.

Preferred R3 groups for parasite treatment and prophylaxis include 2-methylpropen-1-yl; or isobutyl or benzyl, especially with the stereochemistry corresponding to the side chain of L-leucine and L-phenylalanine.

Preferred R3 groups for cathepsin K inhibition include the sidechain corresponding to L-leucine.

The R5 substituent confers many beneficial qualities to molecules of general formula (II) including improvements in potency and offers the potential to append inhibitor molecules with a basic functionality to improve solubility and pharmacokinetic properties. It should be remembered that many cathepsins such as cathepsin K and falcipain are active in acidic vacuoles or physiological microenvironments which may favour basic functionality at this position Additionally, molecules of formula (IV) where R5 is alkyl or other substituent and not simply hydrogen tend to show good chiral stability at the furanone (or corresponding for the pyranone) α-carbon (denoted ring position 4 or C4 herein, unless the context requires otherwise). By chirally stable is meant that the compounds of the invention exist as a predominant stereoisomer rather than an equal mixture of stereoisomers differing in stereochemistry at C4. Preferably the compounds of the invention are at least 90% diastereomically pure.

Note particularly the presence of the substituent R5 in formula (II) in comparison with the absence of any substituent in the same position in formula (I) according to WO 98/50533, WO 98/46582, WO99/64399, WO00/29408, WO00/38687 and WO00/49011.

Interesting compounds of formula II, particularly in the context of cathepsin K inhibition include:

N-[3-Methyl-1S-(2R-methyl-4-oxo-tetrahydro-furan-3S-ylcarbamoyl)-butyl]-2-phenethyl-benzamide

Benzofuran-2-carboxylic-acid-[3-methyl-1S-(2R-methyl-4-oxo-tetrahydro-furan-3S-ylcarbamoyl)-pentyl]-amide

Benzofuran-2-carboxylic acid [1S-(2R-methyl-4-oxo-tetrahydro-furan-3S-ylcarbamoyl)-cyclohexyl]-amide

Benzofuran-2-carboxylic-acid-[1S-(2R-methyl-4-oxo-tetrahydro-furan-3S-ylcarbamoyl)-cyclopentyl]-amide

Naphthalene-2-carboxylic-acid-[1S-(2R-methyl4-oxo-tetrahydro-furan-3S-ylcarbamoyl)-cyclohexyl-amide

Benzofuran-2-carboxylic-acid-[2-cyclopropyl-1S-(2R-methyl-4-oxo-tetrahydro-furan-3S-ylcarbamoyl)-ethyl]-amide

N-[3-Methyl-1S-(2R-methyl-4-oxo-tetrahydro-furan-3S-ylcarbamoyl)-butyl]-4-pyrrol-1-yl-benzamide

N-[3-Methyl-1S-(2R-methyl-4-oxo-tetrahydro-furan-3S-ylcarbamoyl)-butyl]-4-piperidin-1-yl-benzamide

N-[3-Methyl-1S-(2R-methyl-4-oxo-tetrahydro-furan-3S-ylcarbamoyl)-butyl]-4-morpholin-4-yl-benzamide

N-[3-Methyl-1S-(2R-methyl-4-oxo-tetrahydro-furan-3S-ylcarbamoyl)-butyl]-4-piperazin-1-yl-benzamide

N-[3-Methyl-1S-(2R-methyl-4-oxo-tetrahydro-furan-3S-ylcarbamoyl)-butyl]-4-(4-methyl-piperazin-1-yl)-benzamide

N-[3-Methyl-1S-(2R-methyl-4-oxo-tetrahydro-furan-3S-ylcarbamoyl)-butyl]-4-pyrrolidin-1-yl-benzamide

4-(3,3-Dimethyl-piperazin-1-yl)-N-[3-methyl-1S-(2R-methyl-4-oxo-tetrahydro-furan-3S-ylcarbamoyl)-butyl]-benzamide

4-(2,2-Dimethyl-piperazin-1-yl)-N-[3-methyl-1S-(2R-methyl-4-oxo-tetrahydro-furan-3S-ylcarbamoyl)-butyl]-benzamide

4-(4-Allyl-piperazin-1-yl)-N-[3-methyl-1S-(2R-methyl-4-oxo-tetrahydro-furan-3S-ylcarbamoyl)-butyl]-benzamide

4-(4-Cyclopropylmethyl-piperazin-1-yl)-N-[3-methyl-1S-(2R-methyl-4-oxo-tetrahydro-furan-3S-ylcarbamoyl)-butyl]-benzamide

1,2,3,4-Tetrahydro-quinoline-6-carboxylic-acid-[3-methyl-1S-(2R-methyl-4-oxo-tetrahydro-furan-3S-ylcarbamoyl)-butyl]-amide

Benzothiazole-5-carboxylic-acid-[3-methyl-1S-(2R-methyl-4-oxo-tetrahydro-furan-3S-ylcarbamoyl)-butyl]-amide

4-Azepan-1-yl-N-[3-methyl-1S-(2R-methyl-4-oxo-tetrahydro-furan-3S-ylcarbamoyl)-butyl]-benzamide

4-[1,4]Diazepan-1-yl-N-[3-methyl-1S-(2R-methyl-4-oxo-tetrahydro-furan-3S-ylcarbamoyl)-butyl]-benzamide

4-(2-Methylamino-ethylamino)-N-[3-methyl-1S-(2R-methyl-4-oxo-tetrahydro-furan-3S-ylcarbamoyl)-butyl]-benzamide

Naphthalene-1-carboxylic-acid-[3-methyl-1S-(2R-methyl-4-oxo-tetrahydro-furan-3S-ylcarbamoyl)-butyl]-amide

Benzofuran-2-carboxylic-acid-[3-methyl-1S-(2R-methyl-4-oxo-tetrahydro-furan-3S-ylcarbamoyl)-butyl]-amide

Benzo[b]thiophene-2-carboxylic-acid-[3-methyl-1S-(2R-methyl-4-oxo-tetrahydro-furan-3S-ylcarbamoyl)-butyl]-amide

5-Methoxy-benzofuran-2-carboxylic-acid-[3-methyl-1S-(2R-methyl-4-oxo-tetrahydro-furan-3S-ylcarbamoyl)-butyl]-amide

5-Methoxy-benzofuran-2-carboxylic-acid-[3-methyl-1S-(2R-methyl-4-oxo-tetrahydro-furan-3-ylcarbamoyl)-but-3S-enyl]-amide

4-Acetylamino-N-[3-methyl-1S-(2R-methyl-4-oxo-tetrahydro-furan-3S-ylcarbamoyl)-butyl]-benzamide

N-[3-Methyl-1S-(2R-methyl-4-oxo-tetrahydro-furan-3S-ylcarbamoyl)-butyl]-4-morpholin-4-ylmethyl-benzamide

N-[3-Methyl-1S-(2R-methyl-4-oxo-tetrahydro-furan-3S-ylcarbamoyl)-butyl]-4-piperidin-1-ylmethyl-benzamide

Piperidine-1-carboxylic-acid-{4-[3-methyl-1S-(2R-methyl-4-oxo-tetrahydro-furan-3S-ylcarbamoyl)-butylcarbamoyl]-phenyl}-amide

N-[3-Methyl-1S-(2R-methyl-4-oxo-tetrahydro-furan-3S-ylcarbamoyl)-but-3-enyl]-N′-phenyl-terephthalamide

N-[3-Methyl-1S-(2R-methyl-4-oxo-tetrahydro-furan-3S-ylcarbamoyl)-butyl]-N′-phenyl-terephthalamide

N-Ethyl-N′-[3-methyl-1S-(2R-methyl-4-oxo-tetrahydro-furan-3S-ylcarbamoyl)-but-3-enyl]-terephthalamide

N-Ethyl-N′-[3-methyl-1S-(2R-methyl-4-oxo-tetrahydro-furan-3S-ylcarbamoyl)-butyl]-terephthalamide

4-Hydroxy-N-[3-methyl-1S-(2R-methyl-4-oxo-tetrahydro-furan-3S-ylcarbamoyl)-butyl]-3-morpholin-4-ylmethyl-benzamide

4-Hydroxy-N-[3-methyl-1S-(2R-methyl-4-oxo-tetrahydro-furan-3S-ylcarbamoyl)-but-3-enyl]-3-morpholin-4-ylmethyl-benzamide

Biphenyl-4-carboxylic-acid-[3-methyl-1S-(2R-methyl-4-oxo-tetrahydro-furan-3S-ylcarbamoyl)-butyl]-amide

4-tert-Butyl-N-[3-methyl-1S-(2R-methyl4-oxo-tetrahydro-furan-3S-ylcarbamoyl)-butyl]-benzamide

4-tert-Butyl-N-[3-methyl-1S-(2R-methyl-4-oxo-tetrahydro-furan-3S-ylcarbamoyl)-but-3-enyl]-benzamide

4-Guanidino-N-[3-methyl-1S-(2R-methyl-4-oxo-tetrahydro-furan-3S-ylcarbamoyl)-butyl]-benzamide

4-Guanidino-N-[3-methyl-1S-(2R-methyl-4-oxo-tetrahydro-furan-3S-ylcarbamoyl)-but-3-enyl]-benzamide

5-(2-Morpholin-4-yl-ethoxy)-benzofuran-2-carboxylic-acid-[3-methyl-1S-(2R-methyl-4-oxo-tetrahydro-furan-3S-ylcarbamoyl)-butyl]-amide

5-(2-Morpholin-4-yl-ethoxy)-benzofuran-2-carboxylicacid-[3-methyl-1S-(2R-methyl-4-oxo-tetrahydro-furan-3S-ylcarbamoyl)-but-3-enyl]-amide

Naphthalene-2-carboxylic-acid-[3-methyl-1S-(2R-methyl-4-oxo-tetrahydro-furan-3S-ylcarbamoyl)-butyl]-amide

4-Benzenesulfonylamino-N-[3-methyl-1S-(2R-methyl-4-oxo-tetrahydro-furan-3S-ylcarbamoyl)-butyl]-benzamide

3,4,5,6-Tetrahydro-2H-[1,4′]bipyridinyl-4-carboxylic-acid-[3-methyl-1S-(2R-methyl-4-oxo-tetrahydro-furan-3S-ylcarbamoyl)-butyl]-amide

4-(1-Methyl-4,5-dihydro-1H-imidazol-2-yl)-N-[3-methyl-1S-(2R-methyl-4-oxo-tetrahydro-furan-3S-ylcarbamoyl)-butyl]-benzamide

4-(Benzyl-methyl-amino)-N-[3-methyl-1S-(2R-methyl-4-oxo-tetrahydro-furan-3S-ylcarbamoyl)-butyl]-benzamide

N-[3-Methyl-1S-(2R-methyl-4-oxo-tetrahydro-furan-3S-ylcarbamoyl)-butyl]-4-phenylamino-benzamide

4-Benzylamino-N-[3-methyl-1S-(2R-methyl-4-oxo-tetrahydro-furan-3S-ylcarbamoyl)-butyl]-benzamide

1-Methyl-1,2,3,4-tetrahydro-quinoline-6-carboxylic-acid-[3-methyl-1S-(2R-methyl-4-oxo-tetrahydro-furan-3S-ylcarbamoyl)-butyl]-amide

and the corresponding R5 hydroxymethyl compounds;

and pharmaceutically acceptable salts thereof

Additional preferred compounds include

N-[3-Methyl-1S-(3S-methyl-5-oxo-tetrahydro-pyran-4S-ylcarbamoyl)-butyl]-4-pyrrolidin-1-yl-benzamide

N-[3-Methyl-1S-(3S-methyl-5-oxo-tetrahydro-pyran-4S-ylcarbamoyl)-butyl]-4-piperidin-1-yl-benzamide

N-[3-Methyl-1S-(3S-methyl-5-oxo-tetrahydro-pyran-4S-ylcarbamoyl)-butyl]-4-morpholin-4-yl-benzamide

N-[3-Methyl-1S-(3S-methyl-5-oxo-tetrahydro-pyran-4S-ylcarbamoyl)-butyl]-4-(4-methyl-piperazin-1-yl )-benzamide

N-[3-Methyl-1S-(3S-methyl-5-oxo-tetrahydro-pyran-4S-ylcarbamoyl)-butyl]-4-(2-morpholin-4-yl-ethylamino)-benzamide

N-[3-Methyl-1S-(3S-methyl-5-oxo-tetrahydro-pyran-4S-ylcarbamoyl)-butyl]-4-piperazin-1-yl-benzamide

N-[3-Methyl-1S-(3S-methyl-5-oxo-tetrahydro-pyran-4S-ylcarbamoyl)-butyl]-4-[(piperidin-4-ylmethyl)-amino]-benzamide

4-Hydroxy-N-[3-methyl-1S-(3S-methyl-5-oxo-tetrahydro-pyran-4S-ylcarbamoyl)-butyl]-3-morpholin-4-ylmethyl-benzamide

N-[3-Methyl-1S-(3S-methyl-5-oxo-tetrahydro-pyran-4S-ylcarbamoyl)-butyl]-3-pyrrolidin-1-yl-benzamide

N-[3-Methyl-1S-(3S-methyl-5-oxo-tetrahydro-pyran-4S-ylcarbamoyl)-butyl]-3-piperidin-1-yl-benzamide

N-[3-Methyl-1S-(3S-methyl-5-oxo-tetrahydro-pyran-4S-ylcarbamoyl)-butyl]-3-morpholin-4-yl-benzamide

N-[3-Methyl-1S-(3S-methyl-5-oxo-tetrahydro-pyran-4S-ylcarbamoyl)-butyl]-3-(4-methyl-piperazin-1-yl)-benzamide

N-[3-Methyl-1S-(3S-methyl-5-oxo-tetrahydro-pyran-4S-ylcarbamoyl)-butyl]-3-(2-morpholin-4-yl-ethylamino)-benzamide

N-[3-Methyl-1S-(3S-methyl-5-oxo-tetrahydro-pyran-4S-ylcarbamoyl)-butyl]-3-piperazin-1-yl-benzamide

N-[3-Methyl-1S-(3S-methyl-5-oxo-tetrahydro-pyran-4S-ylcarbamoyl)-butyl]-3-[(piperidin-4-ylmethyl)-amino]-benzamide

N-[1S-(3S-Ethyl-5-oxo-tetrahydro-pyran-4S-ylcarbamoyl)-3-methyl-butyl]-4-pyrrolidin-1-yl-benzamide

N-[1S-(3S-Ethyl-5-oxo-tetrahydro-pyran-4S-ylcarbamoyl)-3-methyl-butyl]-4-piperidin-1-yl-benzamide

N-[1S-(3S-Ethyl-5-oxo-tetrahydro-pyran-4S-ylcarbamoyl)-3-methyl-butyl]-4-morpholin-4-yl-benzamide

N-[1S-(3S-Ethyl-5-oxo-tetrahydro-pyran-4S-ylcarbamoyl)-3-methyl-butyl]-4-(4-methyl-piperazin-1-yl)-benzamide

N-[1S-(3S-Ethyl-5-oxo-tetrahydro-pyran-4S-ylcarbamoyl)-3-methyl-butyl]-4-(2-morpholin-4-yl-ethylamino)-benzamide

N-[1S-(3S-Ethyl-5-oxo-tetrahydro-pyran-4S-ylcarbamoyl)-3-methyl-butyl]-4-piperazin-1-yl-benzamide

N-[1S-(3S-Ethyl-5-oxo-tetrahydro-pyran-4S-ylcarbamoyl)-3-methyl-butyl]-4-[(piperidin-4-ylmethyl)-amino]-benzamide

N-[1S-(3S-Ethyl-5-oxo-tetrahydro-pyran-4S-ylcarbamoyl)-3-methyl-butyl]-4-hydroxy-3-morpholin-4-ylmethyl-benzamide

N-[1S-(3S-Ethyl-5-oxo-tetrahydro-pyran-4S-ylcarbamoyl)-3-methyl-butyl]-3-pyrrolidin-1-yl-benzamide

N-[1S-(3S-Ethyl-5-oxo-tetrahydro-pyran-4S-ylcarbamoyl)-3-methyl-butyl]-3-piperidin-1-yl-benzamide

N-[1S-(3S-Ethyl-5-oxo-tetrahydro-pyran-4S-ylcarbamoyl)-3-methyl-butyl]-3-morpholin-4-yl-benzamide

N-[1S-(3S-Ethyl-5-oxo-tetrahydro-pyran-4S-ylcarbamoyl)-3-methyl-butyl]-3-(4-methyl-piperazin-1-yl)-benzamide

N-[1S-(3S-Ethyl-5-oxo-tetrahydro-pyran-4S-ylcarbamoyl)-3-methyl-butyl]-3-(2-morpholin-4-yl-ethylamino)-benzamide

N-[1S-(3S-Ethyl-5-oxo-tetrahydro-pyran-4S-ylcarbamoyl)-3-methyl-butyl]-3-piperazin-1-yl-benzamide

N-[1S-(3S-Ethyl-5-oxo-tetrahydro-pyran-4S-ylcarbamoyl)-3-methyl-butyl]-3-[(piperidin-4-ylmethyl)-amino]-benzamide

N-[3-Methyl-1S-(3S-oxo-5-propyl-tetrahydro-pyran-4S-ylcarbamoyl)-butyl]-4-pyrrolidin-1-yl-benzamide

N-[3-Methyl-1S-(3S-oxo-5-propyl-tetrahydro-pyran-4S-ylcarbamoyl)-butyl]-4-piperidin-1-yl-benzamide

N-[3-Methyl-1S-(3S-oxo-5-propyl-tetrahydro-pyran-4S-ylcarbamoyl)-butyl]-4-morpholin-4-yl-benzamide

N-[3-Methyl-1S-(3S-oxo-5-propyl-tetrahydro-pyran-4S-ylcarbamoyl)-butyl]-4-(4-methyl-piperazin-1-yl)-benzamide

N-[3-Methyl-1S-(3S-oxo-5-propyl-tetrahydro-pyran-4S-ylcarbamoyl)-butyl]-4-(2-morpholin-4-yl-ethylamino)-benzamide

N-[3-Methyl-1S-(3S-oxo-5-propyl-tetrahydro-pyran-4S-ylcarbamoyl)-butyl]-4-piperazin-1-yl-benzamide

N-[3-Methyl-1S-(3S-oxo-5-propyl-tetrahydro-pyran-4S-ylcarbamoyl)-butyl]-4-[(piperidin-4-ylmethyl)-amino]-benzamide

4-Hydroxy-N-[3-methyl-1S-(3S-oxo-5-propyl-tetrahydro-pyran-4S-ylcarbamoyl)-butyl]-3-morpholin-4-ylmethyl-benzamide

N-[3-Methyl-1S-(3S-oxo-5-propyl-tetrahydro-pyran-4S-ylcarbamoyl)-butyl]-3-pyrrolidin-1-yl-benzamide

N-[3-Methyl-1S-(3S-oxo-5-propyl-tetrahydro-pyran-4S-ylcarbamoyl)-butyl]-3-piperidin-1-yl-benzamide

N-[3-Methyl-1S-(3S-oxo-5-propyl-tetrahydro-pyran-4S-ylcarbamoyl)-butyl]-3-morpholin-4-yl-benzamide

N-[3-Methyl-1S-(3S-oxo-5-propyl-tetrahydro-pyran-4S-ylcarbamoyl)-butyl]-3-(4-methyl-piperazin-1-yl)-benzamide

N-[3-Methyl-1S-(3S-oxo-5-propyl-tetrahydro-pyran-4S-ylcarbamoyl)-butyl]-3-(2-morpholin-4-yl-ethylamino)-benzamide

N-[3-Methyl-1S-(3S-oxo-5-propyl-tetrahydro-pyran-4S-ylcarbamoyl)-butyl]-3-piperazin-1-yl-benzamide

N-[3-Methyl-1S-(3S-oxo-5-propyl-tetrahydro-pyran-4S-ylcarbamoyl)-butyl]-3-[(piperidin-4-ylmethyl)-amino]-benzamide;

the corresponding 3R,4R stereoisomers of the respective compounds enumerated above;

and pharmaceutically acceptable salts thereof

The compounds of the invention can form salts which form an additional aspect of the invention. Appropriate pharmaceutically acceptable salts of the compounds of Formula II include salts of organic acids, especially carboxylic acids, including but not limited to acetate, trifluoroacetate, lactate, gluconate, citrate, tartrate, maleate, malate, pantothenate, isethionate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-napthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids. The compounds of Formula II may in some cases be isolated as the hydrate.

It will be appreciated that the invention extends to prodrugs solvates, complexes and other forms releasing a compound of formula II in vivo.

While it is possible for the active agent to be administered alone, it is preferable to present it as part of a pharmaceutical formulation. Such a formulation will comprise the above defined active agent together with one or more acceptable carriers/excipients and optionally other therapeutic ingredients. The carrier(s) must be acceptable in the sense of being compatible with the other ingredients of the formulation and not deleterious to the recipient.

The formulations include those suitable for rectal, nasal, topical (including buccal and sublingual), vaginal or parenteral (including subcutaneous, intramuscular, intravenous and intradermal) administration, but preferably the formulation is an orally administered formulation. The formulations may conveniently be presented in unit dosage form, e.g. tablets and sustained release capsules, and may be prepared by any methods well known in the art of pharmacy.

Such methods include the step of bringing into association the above defined active agent with the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of Formula II or its pharmaceutically acceptable salt in conjunction or association with a pharmaceutically acceptable carrier or vehicle. If the manufacture of pharmaceutical formulations involves intimate mixing of pharmaceutical excipients and the active ingredient in salt form, then it is often preferred to use excipients which are non-basic in nature, i.e. either acidic or neutral.

Formulations for oral administration in the present invention may be presented as discrete units such as capsules, cachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion and as a bolus etc.

With regard to compositions for oral administration (e.g. tablets and capsules), the term suitable carrier includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring or the like can also be used.

It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

The appropriate dosage for the compounds or formulations of the invention will depend upon the indication and the patient and is readily determined by conventional animal trials. Dosages providing intracellular (for inhibition of physiological proteases of the papain superamily) concentrations of the order 0.01-100 μM, more preferably 0.01-10 μM, such as 0.1-25μM are typically desirable and achievable. Ex vivo or topical administration against parasites will typically involve higher concentrations.

The term “N-protecting group” or “N-protected” and the like as used herein refers to those groups intended to protect the N-terminus of an amino acid or peptide or to protect an amino group against undesirable reactions during synthetic procedures. Commonly used N-protecting groups are disclosed in Greene, “Protective Groups in Organic Synthesis” (John Wiley & Sons, New York, 1981), which is hereby incorporated by reference. N-protecting groups include acyl groups such as formyl, acetyl, propionyl, pivaloyl, t-butylacetyl, 2-chloroacetyl, 2-bromoacetyl, trifluoracetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl, and the like, carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl )-1-methylethoxycarbonyl, α,α-dimethyl-3,5-dimethoxybenzyloxycarbonyl, benzhydryloxycarbonyl, t-butoxycarbonyl, diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl, and the like; alkyl gropus such as benzyl, triphenylmethyl, benzyloxymethyl and the like; and silyl groups such as trimethylsilyl and the like. Favoured N-protecting groups include formyl, acetyl, allyl, Fmoc, benzoyl, pivaloyl, t-butylacetyl, phenylsulfonyl, benzyl, t-butoxycarbonyl (Boc) and benzyloxycarbonyl (Cbz).

Hydroxy and/or carboxy protecting groups are also extensively reviewed in Greene ibid and include ethers such as methyl, substituted methyl ethers such as methoxymethyl, methylthiomethyl, benzyloxymethyl, t-butoxymethyl, 2-methoxyethoxymethyl and the like, silyl ethers such as trimethylsilyl (TMS), t-butyldimethylsilyl (TBDMS) tribenzylsilyl, triphenylsilyl, t-butyldiphenylsilyl (TBDPS), triisopropyl silyl and the like, substituted ethyl ethers such as 1-ethoxymethyl, 1-methyl-1-methoxyethyl, t-butyl, allyl, benzyl, p-methoxybenzyl, dipehenylmethyl, triphenylmethyl and the like, aralkyl groups such as trityl, and pixyl(9-hydroxy-9-phenylxanthene derivatives, especially the chloride). Ester hydroxy protecting groups include esters such as formate, benzylformate, chloroacetate, methoxyacetate, phenoxyacetate, pivaloate, adamantoate, mesitoate, benzoate and the like. Carbonate hydroxy protecting groups include methyl vinyl, allyl, cinnamyl, benzyl and the like.

Compounds of the invention are synthesised by a combination of chemistries, performed either in solution or on the solid phase. The synthesis methodology described in schemes 1-8 of our copending PCT/GB00/01894 but employing the appropriate R′ capping group is convenient for the compounds of the invention.

### Scheme 1. Preparation of dihydro-2(3H)-5-alkyl Furanone Ring System

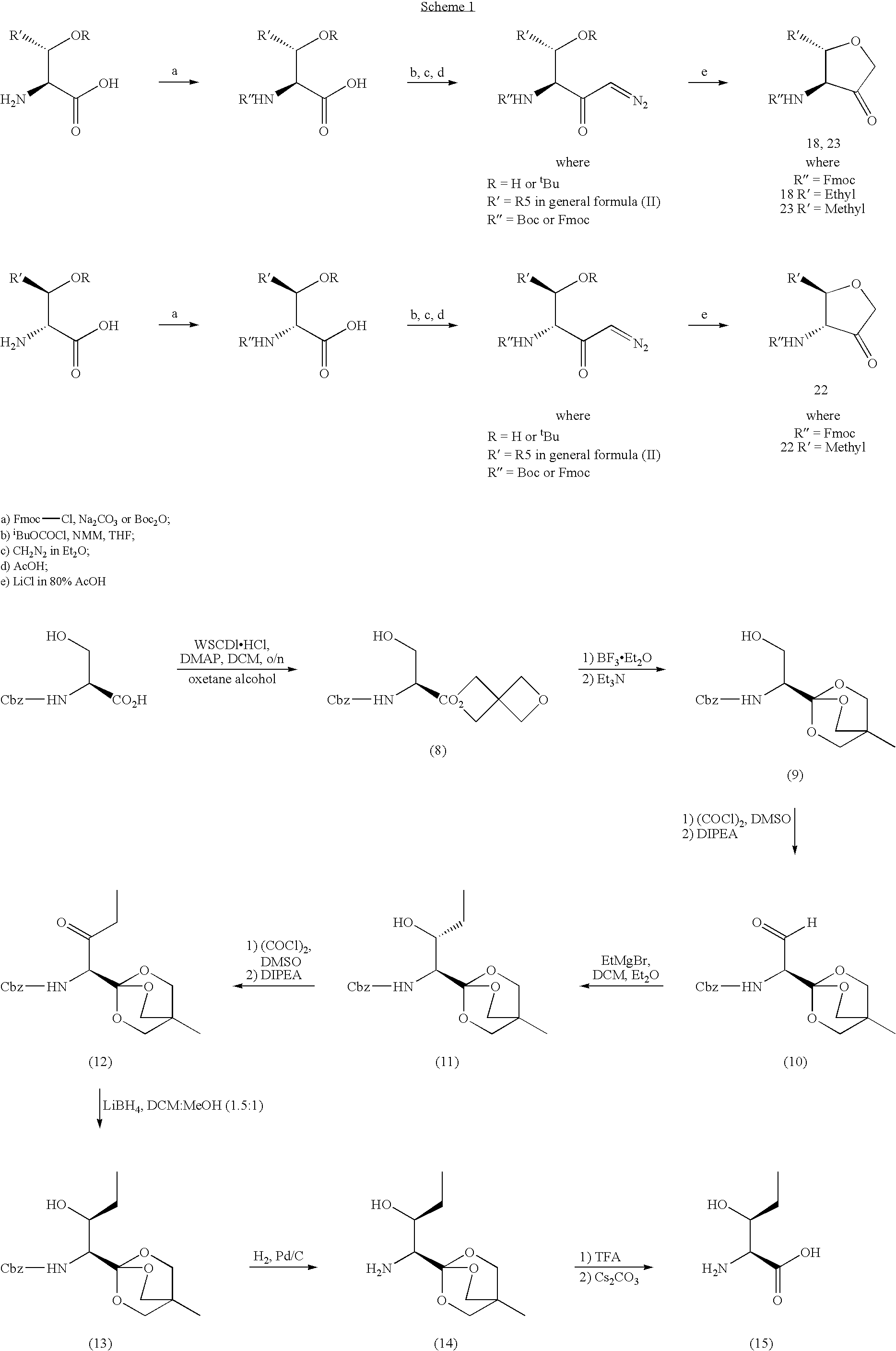

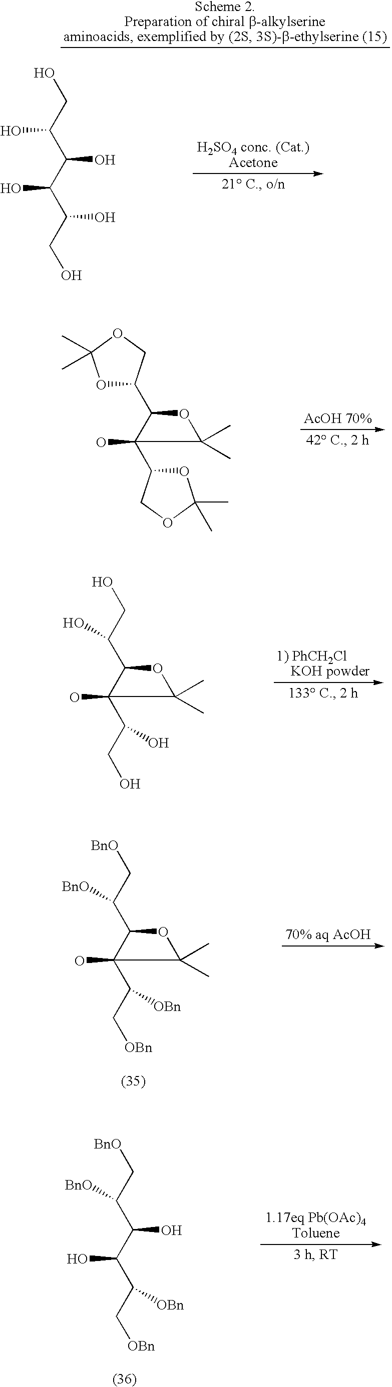

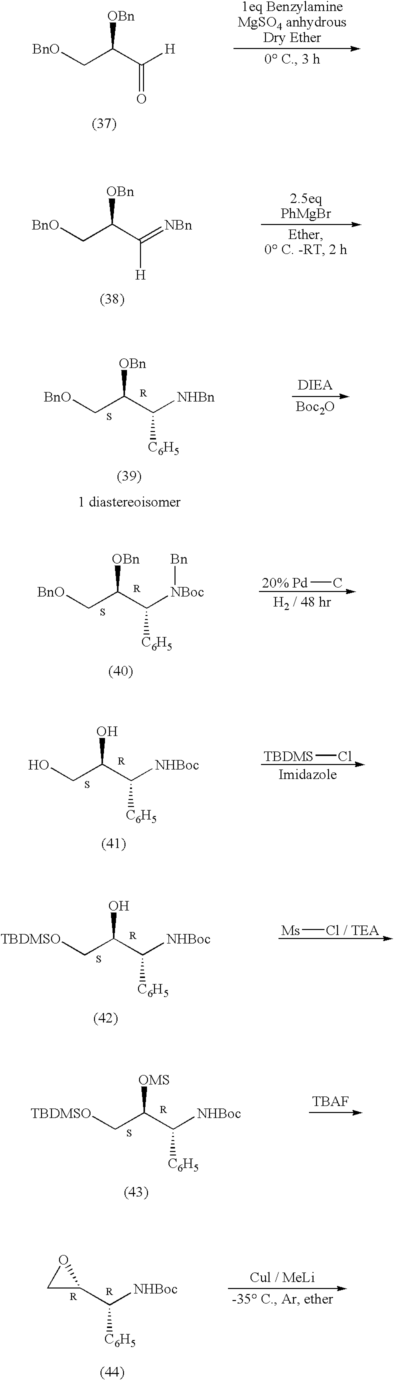

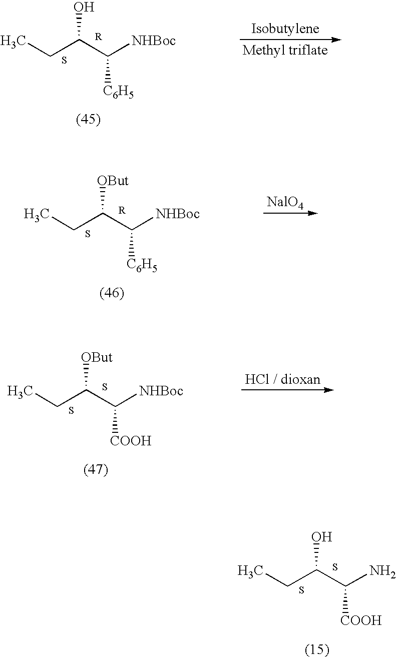

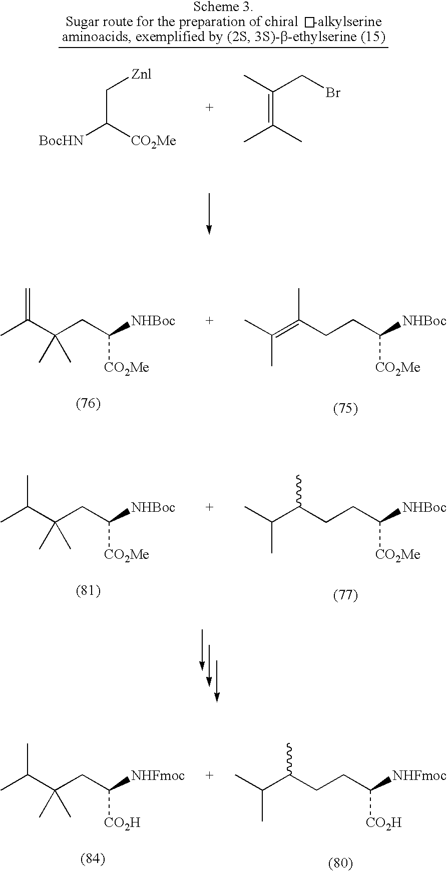

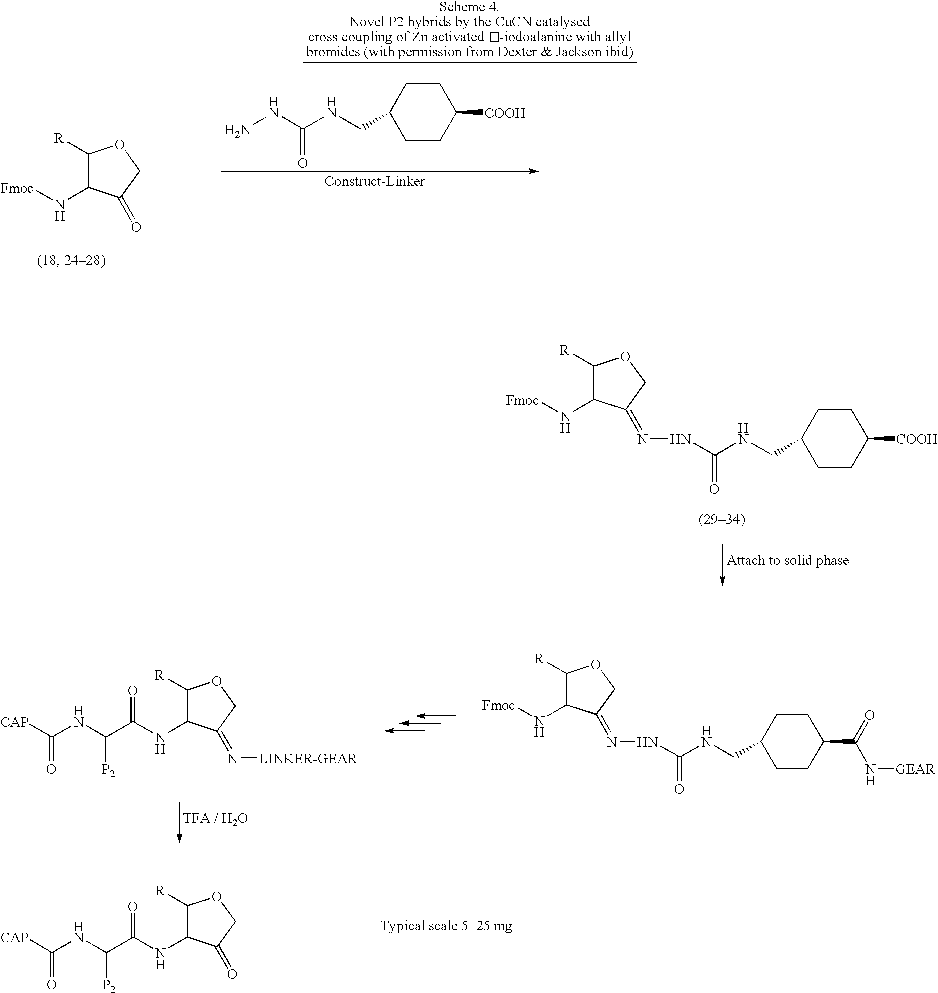

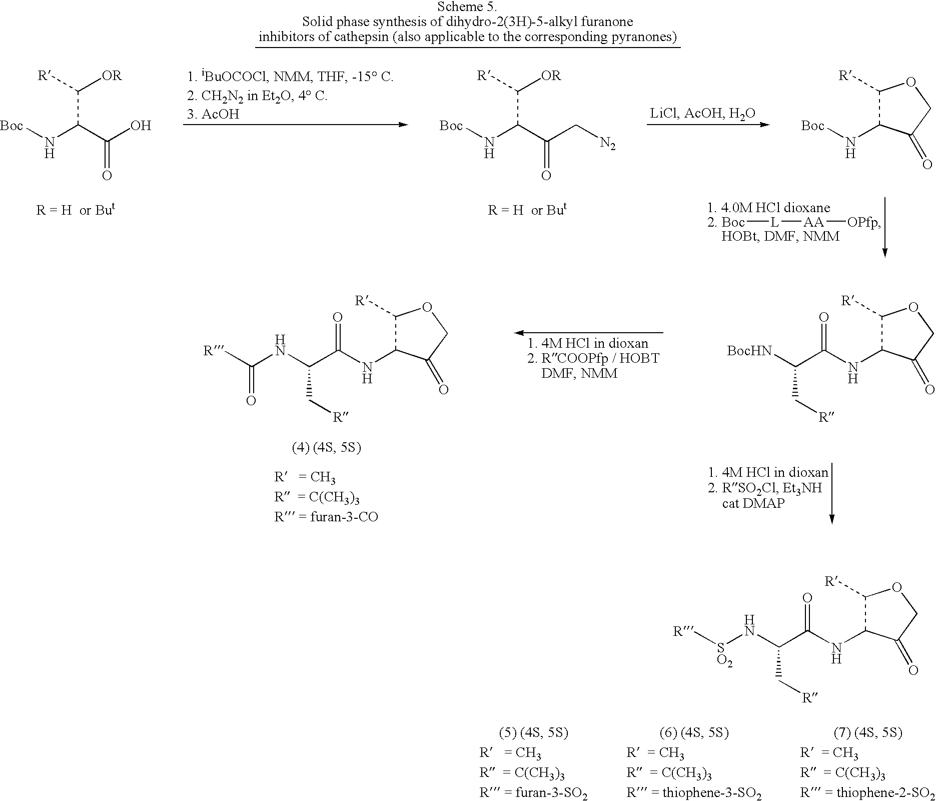

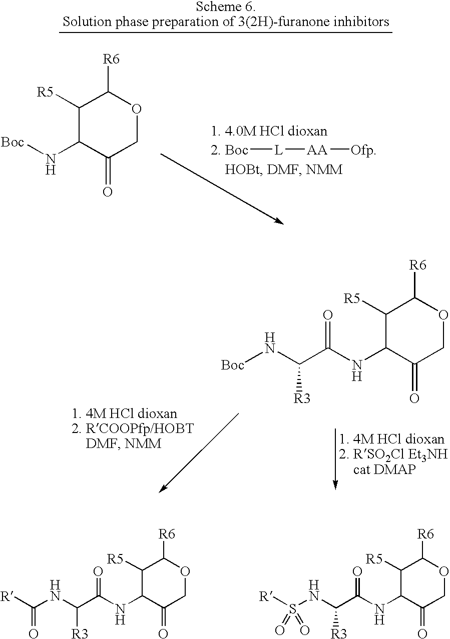

### Scheme 6A solution phase N-terminal extension and capping of a pyranone building block

Additional routes to building blocks include:

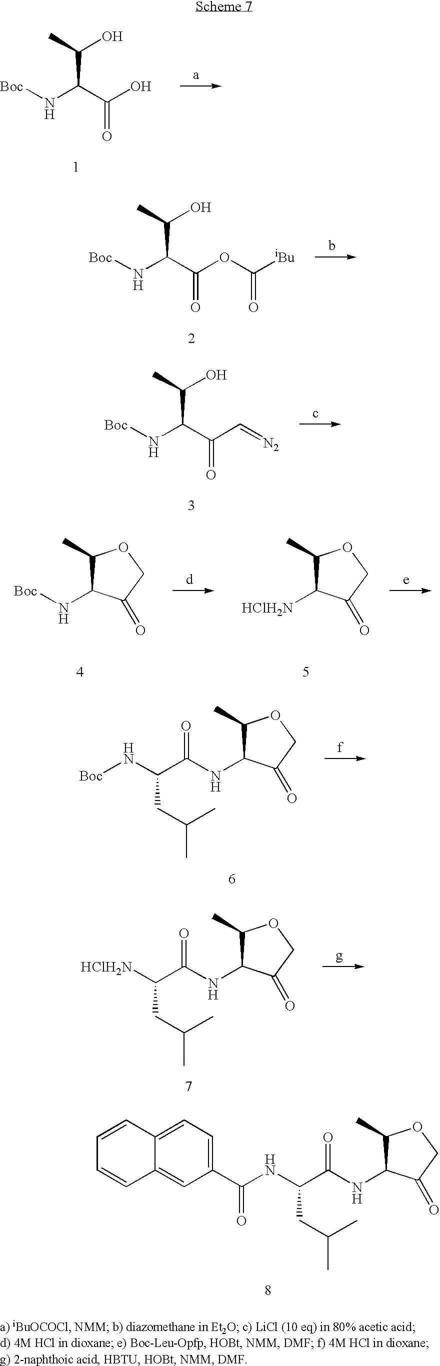

Compounds of the general formula (IV), wherein q=0, are prepared by methods shown in Scheme 7. Activation of the known Boc-aminoacid 1-Scheme-7 with isobutyl chloroformate and 4-methylmorpholine provides 2-Scheme-7. Subsequent treatment of 2-Scheme-7 with diazomethane provides the diazoketone 3-Scheme-7. Cyclization of diazoketone 3-Scheme-7 can be effected by lithium chloride/aqueous acetic acid to give the dihydro-3(2H)-furanone 4-Scheme-7. The tert-butoxycarbonyl group may be removed from 4-Scheme-7, by treatment with acid, and provides the amine salt 5-Scheme-7. The amine salt 5-Scheme-7 may be coupled with a carboxylic acid by methods that are known in the art, such as coupling with a pentafluorophenol derivative in the presence of HOBT and NMM, to provide the amide 6-Scheme-7. The tert-butoxycarbonyl group may be removed from 6-Scheme-7 by treatment with an acid, such as hydrogen chloride in dioxane, to provide the amine salt 7-Scheme-7. The amine salt 7-Scheme-7 may be coupled with a carboxylic acid by methods that are known in the art, such as coupling with an acid in the presence of HBTU and HOBT, to provide the amide 8-Scheme-7.

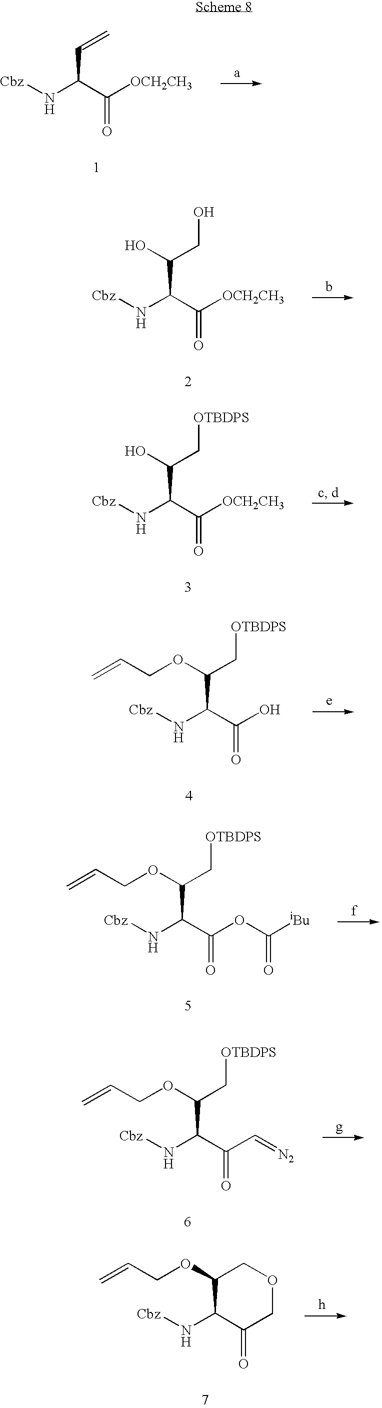

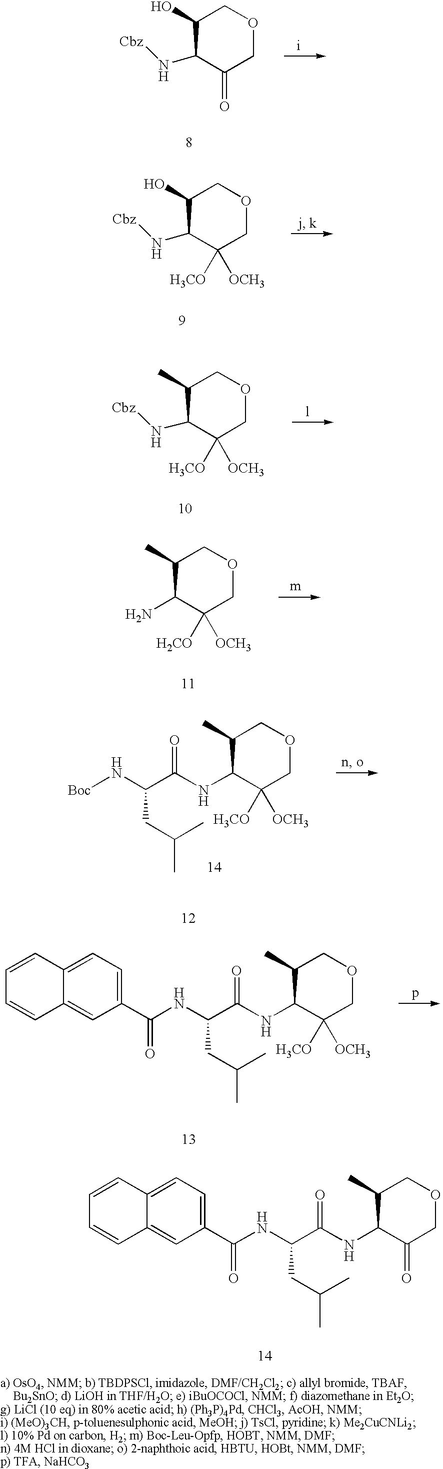

Compounds of the general formula (IV), wherein q=1, are prepared by methods shown in Scheme 8. Treatment of the known Cbz-ethyl ester 1-Scheme-8 with osmium tetroxide and 4-methylmorpholine provides the diol 2-Scheme-8. Protection of the primary alcohol may be effected with tert-butyldiphenylsilylchloride and imidazole to provide 3-Scheme-8. Protection of the secondary alcohol 3-Scheme-8 may be achieved with allyl bromide and subsequent base hydrolysis of the ethyl ester provides 4-Scheme-8. Activation of the acid 4-Scheme-8 may be achieved with isobutyl chloroformate and 4-methylmorpholine to provide 5-Scheme-8. Subsequent treatment of 5-Scheme-8 with diazomethane provides the diazoketone 6-Scheme-8. Cyclization of diazoketone 6-Scheme-8 can be effected by lithium chloride/aqueous acetic acid to give the 3-pyranone 7-Scheme-8. The allyl protection may be removed from 7-Scheme-8, by treatment with palladium(0) and acid, to provide alcohol 8-Scheme-8. Ketal formation from ketone 8-Scheme-8 may be effected by treatment with trimethylorthoformate and p-toluenesulphonic acid to provide 9-Scheme-8. Conversion of the alcohol 9-Scheme-8 to the methyl derivative 10-Scheme 8 can be achieved utilising methods that are known in the art, such as tosylation with tosylchloride and pyridine, with subsequent reaction with the higher order cuprate prepared from methyl lithium. Removal of the Cbz protecting group from 10-Scheme 8 may be achieved with 10% Pd on carbon in the presence of hydrogen to provide 11-Scheme-8. The amine 11-Scheme-8 can be coupled with a carboxylic acid by methods that are known in the art, such as coupling with a pentafluorophenol derivative in the presence of HOBT and NMM, to provide the amide 12-Scheme-8. The tert-butoxycarbonyl group may be removed by treatment with an acid, such as hydrogen chloride in dioxane and the amine salt subsequently coupled with a carboxylic acid by methods that are known in the art, such as coupling with an acid in the presence of HBTU and HOBT, to provide the amide 13-Scheme-8. Removal of the ketal functionality from 13-Scheme-8 may be achieved with trifluoroacetic acid in the presence of sodium hydrogen carbonate to provide 14-Scheme-8.

Compounds of the general formula II wherein q=1 can alternatively be prepared by the methods shown in Scheme 9.

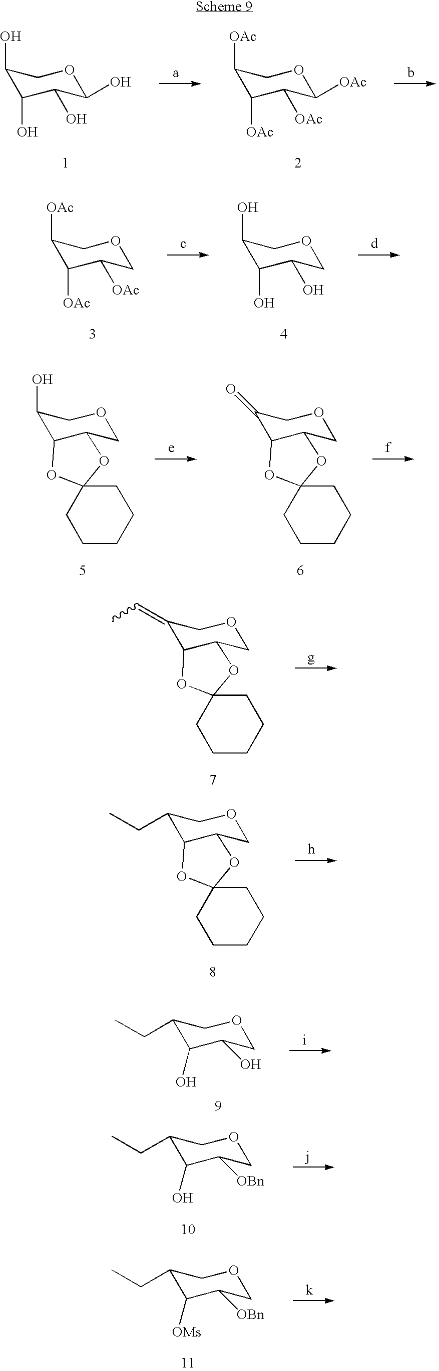

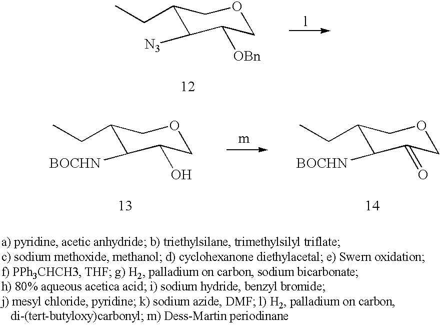

Lyxose 1-scheme-9 can be peracetylated to give 2-scheme-9 with acetic anhydride in pyridine at room temperature overnight. Reduction at the anomeric centre to afford 3-scheme-9 may be achieved using triethylsilane in the presence of trimethylsilyl triflate. Hydrolysis of the triacetate 3-scheme-9 affords 4-scheme-9 whereupon the vicinal diol can be protected as the cyclohexanone acetal 5-scheme-9. Swern oxidation of the unprotected alcohol functionality gives 6-scheme-9, a key intermediate for the introduction of the required C5 pyranone substitution. Ethyl substitution is achieved here by treatment with ethyl triphenylphosphonium bromide with potassium tert-butoxide in THF at 0° C. to produce 7-scheme-9. Hydrogenation of 7-scheme-9 in ethyl acetate with sodium bicarbonate gives the ethyl derivative 8-scheme-9 with the stereochemistry shown. Deprotection of the cyclohexanone acetal 8-scheme-9 can be achieved with aqueous acetic acid overnight to afford the diol 9-scheme-9. Selective benzylation of the equatorial hydroxyl group gives 10-scheme-9, which can then be mesylated using mesyl chloride in pyridine at 50° C. to produce 11-scheme-9. Azide displacement of mesylate anion using sodium azide in DMF at 80° C. affords 12-scheme-9, from which the pyranol 13-scheme-9 can be obtained by hydrogenolysis in the presence of BOC-anhydride. Oxidation to the pyranone 14-scheme-1 is achieved using the Dess-Martin periodinane.

In scheme 9, the C5 substitution is introduced using Wittig chemistry followed by hydrogenation, and hence compound 6-scheme-9 is converted to the C5 ethyl derivative 8-scheme-9. Alternative C5 substitution can be achieved using this route. For example, alternative Wittig or Horner-Emmons chemistry will lead to different alkyl substituents. In an analogous manner, the C5 hydroxymethyl group can be prepared and this itself can be further derivatised to other groups such as halogen, amino and other basic groups and sulfhydryl.

A general methodology starting from L-lyxose has been established for the preparation of various 5-substituted 4-amino 3-hydroxy pyranols with all four possible combinations of configuration at position 4 and 5 i.e. 4S,5S; 4S,5R; 4R,5S and 4R,5R. This methodology is exemplified in Scheme 9A. The pyranols can then be N-extended and capped as described herein and subsequently oxidised to the keto compounds, for example by Dess Martin periodination.

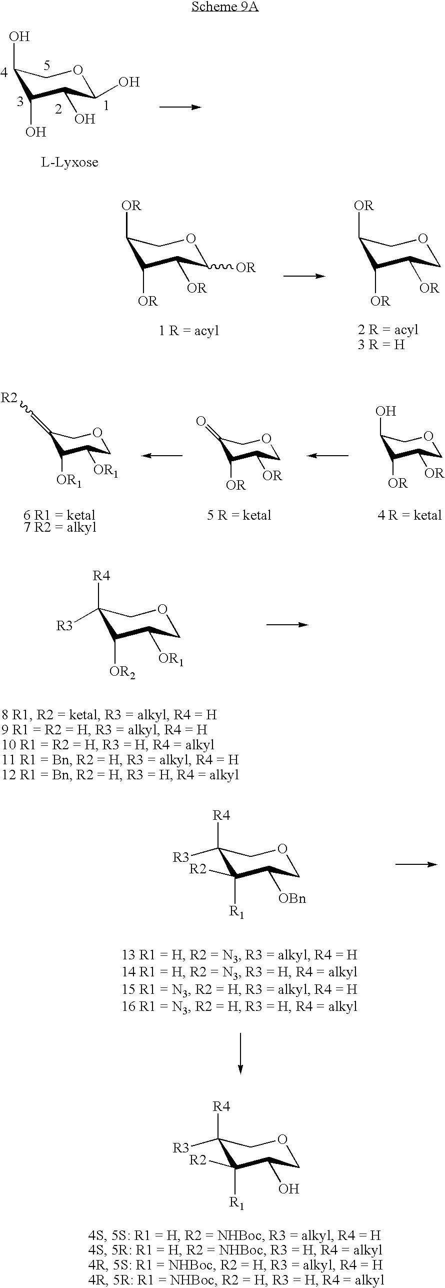

L-Lyxose can be acylated with a suitable acylating agent such as acid anhydride, acyl halide in an organic solvent like pyridine or other mixed organic solvents, to give the peracylated compound 1-scheme-9A. This compound can then be subjected to anomeric reduction with a trialkyl silane together with a Lewis acid such as triethyl silane and trimethylsilyl trifluormethanesulphonate. Transforming the compound into the corresponding halo-, sulpho- or thiocarbo-glycoside followed by a radical reduction, using known methodology, can also bring about the anomeric reduction. Deacylation under basic condition provides the triol 3-scheme-9A, which can be selectively protected on the 2,3-hydroxylgroups forming a ketal 4-scheme 9A by using standard protecting group methodology. Oxidation of the 4-OH group into the keto function 5-scheme-9A can be performed with the Swern procedure, Dess-Martin or any other suitable oxidation method. Various 4-substituted alkenes 6-scheme-9A can be achieved by using appropriate Wittig reagents for example triphenylalkylphosphonium halide or triphenylalkylarylphosphonium halide together with a base. Catalytic hydrogenation of the Wittig product in the presence of a buffer provides predominantly compound 8-scheme-9A. Alternatively, the compound with the other configuration at this position 10-scheme-9A can be obtained by removal of the ketal protecting group prior to the hydrogenation. The alkene compound can also be subjected to hydroboration, which will introduce a hydroxyl group, suitable for further modifications.

Another possibility to achieve the 4-alkyl compounds is to transform the 4-OH group into a leaving group for example a sulphonate followed by displacement by a cuprous or Grignard reagent of the desired alkylgroup.

The ketal protecting group can be removed under acidic conditions such as 1 M HCl/THF 1:1 at room temperature or heating to 80° C. in aqueous acetic acid which will give the diol 8-scheme-9A. Selective protection of the 2-OH group with an alkylating agent such as benzyl halide or any other similar reagent in the presence of a base can give exclusively or predominantly the 2-O-protected compound 11,12-scheme-9A. The 3-OH can be converted to a suitable leaving group such as a sulphonate, which subsequently can be displaced by an azide 13,14-scheme-9A. Alternatively, a Mitsunobu reaction can be used to produce the azide-substituted compound. Hydrogenation of the azide-compound in the presence of a carbamoylating agent like di-tert-butyl dicarbonate provides the desired 1,5-anhydro-3-[(tert-butoxycarbonyl)amino]-3,4-dideoxy-4-ethyl-D-xylitol and 1,5-anhydro-3-[(tert-butoxycarbonyl)amino]-2,3-dideoxy-2-ethyl-L-arabinitol.

The series of compounds with the other configuration at carbon 3 can be prepared by inversion of the configuration of the 3-OH in compound 11,12-scheme-9A by methods that are known in the art, followed by the above procedure i.e. putting on a leaving group and azide displacement. They can also be prepared by the following sequence. Oxidation of the 3-OH into a ketone, using the oxidation reagents previously described, transformation of the ketone into an oxime, utilising reagents such as benzyloxyamine halide and finally reduction of the oxime into the aminofunction. This will provide a mixture of the compounds with the two different configurations, which can be separated using known methodology. Boc-protection of the aminogroup and reductive removal of the benzyl protecting group provides the compounds with the remaining two configurations 4R,5S and 4R,5R.

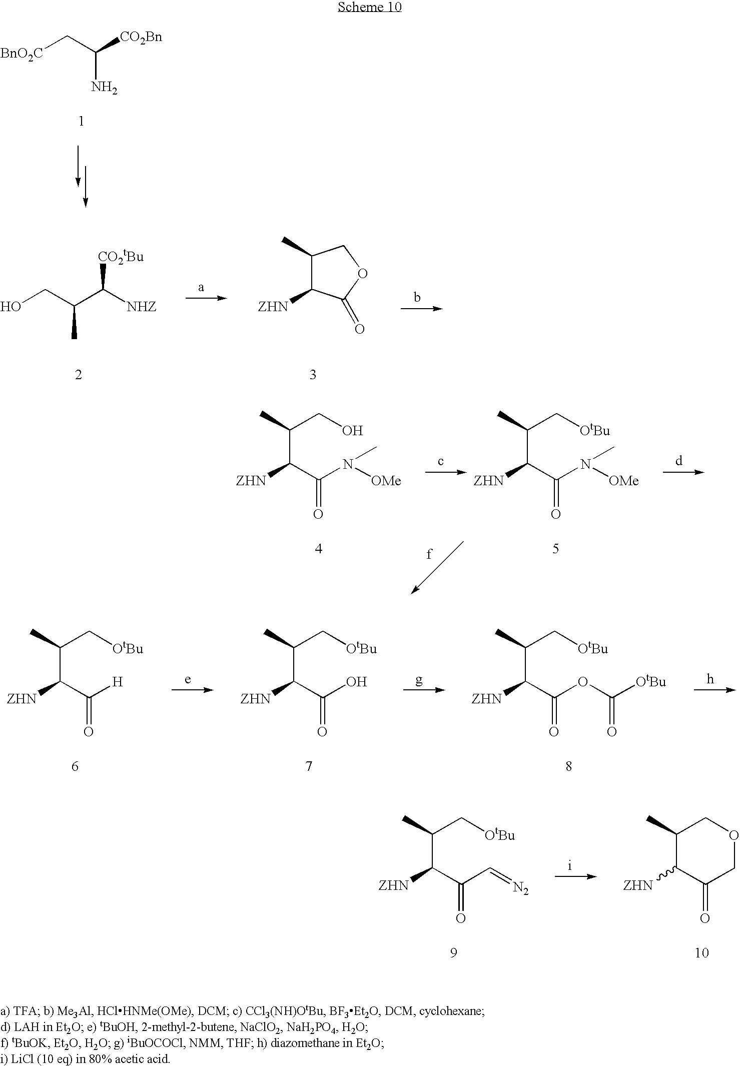

Compounds of the general formula (IV), wherein q is 1 are alternatively prepared by methods shown in Scheme 10. Alcohol 2-Scheme-10 can be prepared following the literature procedure reported by J. E. Baldwin et al (Tetrahedron, 1995, 51 (43), 11581). Removal of the ester functionality from 2-Scheme-10 can be achieved with trifluoroacetic acid to provide the lactone 3-Scheme-10. Lactone 3-Scheme-10 can be ring opened by MeONHMe in the presence of Me₃Al to provide the alcohol 4-Scheme-10. The tert-butoxycarbonyl group may be introduced onto alcohol 4-Scheme-10 to provide 5-Scheme-10. The Weinreb amide 5-Scheme-10 can then be treated with lithium aluminum hydride to provide the aldehyde 6-Scheme-10. Oxidation of the aldehyde 6-Scheme-10 can be effected by sodium chlorite to provide the acid 7-Scheme-2. Alternatively, the Weinreb amide 5-Scheme-10 can then be treated with potassium-tert-butoxide to directly provide the acid 7-Scheme-10. Activation of the acid 7-Scheme-10 with isobutyl chloroformate and 4-methylmorpholine provides 8-Scheme-10. Subsequent treatment of 8-Scheme-10 with diazomethane provides the diazoketone 9-Scheme-10. Cyclization of diazoketone 9-Scheme-10 can be effected by lithium chloride/aqueous acetic acid to give the dihydro-3(2H)-furanone 10-Scheme-10.

In Scheme 10, the C5 substitution of the pyranone is introduced via stereoselective alkylation of a beta-lactam derived from aspartic acid, as outlined by J. E. Balwin et al (Tetrahedron 1995, 51, 11581). Alternative substitution of the pyranone using this methodology can be achieved by varying the electrophilic component in the alkylation step. Hence, various alkyl or aryl-C1-7 alkyl substitutions can be made in this manner.

An alternative ring closing route to compound of Formula II wherein q is 1 is depicted in scheme II below with respect to a model compound wherein the R5 functionality is duplicated:

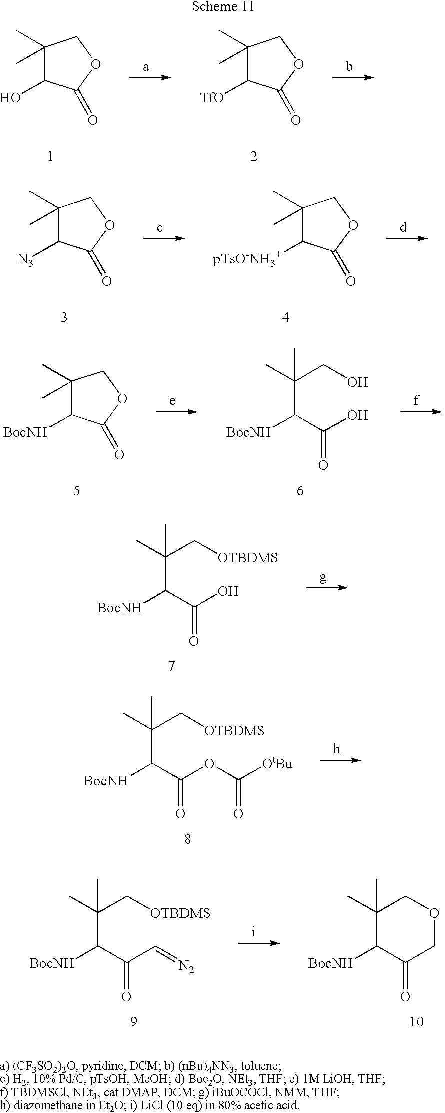

Pantolactone 1-Scheme-11 is commercially available and is first converted to the triflate 2-Scheme-11. The triflate 2-Scheme-11 may be displaced with tetrabutylammonium azide to provide the corresponding azide 3-Scheme-11. Azide 3-Scheme-11 may be reduced to provide the amine salt 4-Scheme-11. Protection of the amine salt 4-Scheme-11 provides 5-Scheme-11. Ring opening of the lactone 5-Scheme-11 with lithium hydroxide provides the acid 6-Scheme-11. Protection of the primary alcohol 6-Scheme-11 with tetrabutyldimethylsilyl chloride in the presence of base provides acid 7-Scheme-11. Activation of the acid 7-Scheme-11 with isobutyl chloroformate and 4-methylmorpholine provides 8-Scheme-11. Subsequent treatment of 8-Scheme-11 with diazomethane provides the diazoketone 9-Scheme-11. Cyclization of diazoketone 9-Scheme-11 can be effected by lithium chloride/aqueous acetic acid to give the model dihydro-3(2H)-pyranone 10-Scheme-11. The ring closing methodology demonstrated in this example is also applicable to compounds of formula II with various R5 functionalities.

to 5-methyl and ethyl furanones as building blocks toward inhibitors or as intermediates to access other R5 functionalities are as shown in schemes 12 and 13.

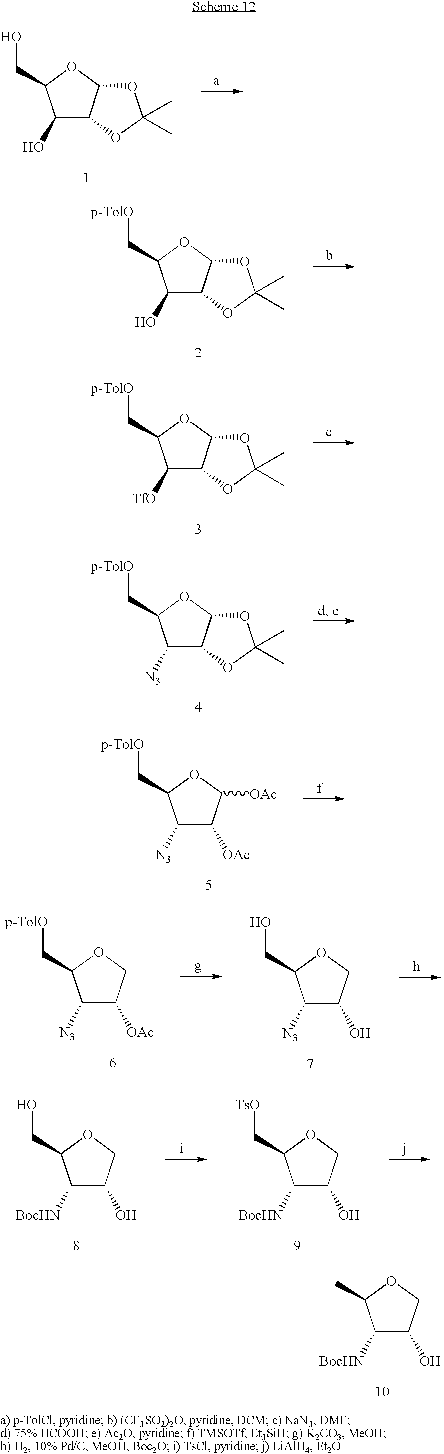

An alternative synthesis of methyl furanones is shown in Scheme 12. 1,2-Isopropylidene-D-xylofuranoside 1-Scheme-12 is first converted to the p-toluoyl ester 2-Scheme-12 with p-toluoyl chloride and pyridine. The secondary alcohol 3-Scheme-12 may be converted to the triflate 3-Scheme-12. The triflate 3-Scheme-12 may be displaced with sodium azide to provide the corresponding azide 4-Scheme-12. Deprotection of the 1,2-isopropylidene of 4-Scheme-12 and subsequent acetylation of the residue provides diacetate 5-Scheme-12. Reduction of the anomeric centre of 5-Scheme-12 with trimethylsilyl triflate and triethylsilane provides monoacetate 6-Scheme-12. Removal of the two ester groups from 6-Scheme-12 with potassium carbonate affords alcohol 7-Scheme-12. Reduction of the azide 7-Scheme-12 in the presence of Boc anhydride affords the key intermediate furanol 8-Scheme-12. Furanol 8-Scheme-12 can be transformed to the methyl furanol 10-Scheme-12 by converting the primary alcohol functionality of 8-Scheme-12 to the tosylate 9-Scheme-12. which in turn can be reduced with lithium aluminium hydride to provide the methyl furanol 10-Scheme-12. As described herein, furanol 10-Scheme-12 can be used to build up inhibitors of the invention in solution or on solid phase. Solid phase chemistry would typically require conversion of the Boc protection to Fmoc chemistry. The ultimate synthetic step involves oxidation of the furanol functionality to the corresponding furanone using an oxidant such as Dess-Martin periodinane. Alternatively, the oxidation may be carried out prior to subsequent modifications at the N-terminus. Importantly, furanol 8-Scheme-12 also provides an opportunity for introduction of diverse functionality at C-5 as the hydroxmethylene can be used for subsequent transformations known to those skilled in the art.

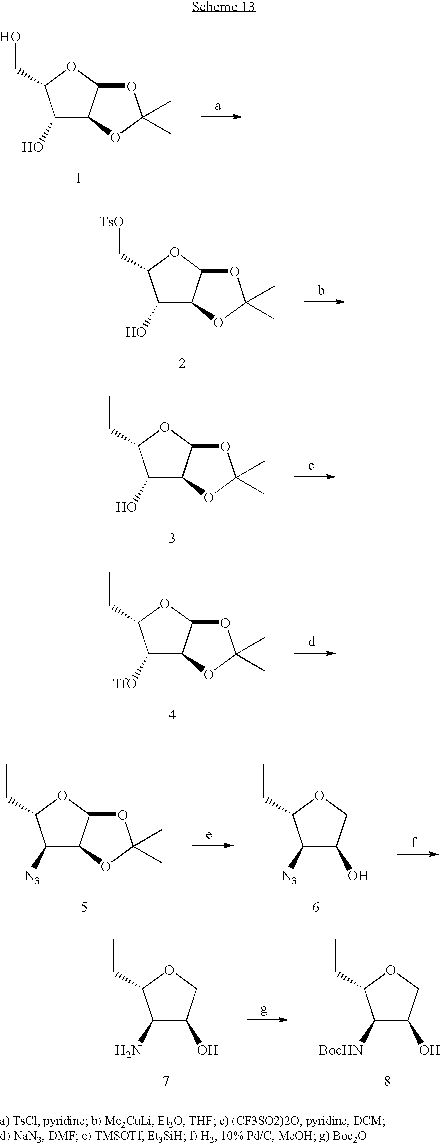

An alternative synthesis of ethyl furanones is shown in Scheme 13. 1,2-Isopropylidene-L-xylofuranoside 1-Scheme-13 is used as the starting material and is first converted to the tosylate 2-Scheme-13. The tosylate 2-Scheme-13 is readily displaced using cuprate chemistry to provide the ethyl furanoside 3-Scheme-13. The secondary alcohol 3-Scheme-13 may be converted to the triflate 4-Scheme-13 using triflic anhydride and pyridine. The triflate 4-Scheme-13 may be displaced with sodium azide to provide the corresponding azide 5-Scheme-13. Reduction of the anomeric centre of 5-Scheme-13 with trimethylsilyl triflate and triethylsilane provides alcohol 6-Scheme-13. Reduction of the azide 6-Scheme-13 with hydrogen in the presence of 10% palladium on carbon provides amine 7-Scheme-13. Protection of the amine 7-Scheme-13 with Boc anhydride provides the ethyl furanol 8-Scheme-13. As described previously, furanol 8-Scheme-13 can be used to build up potential inhibitors in solution or on solid phase. Solid phase chemistry would require conversion of the Boc protection to Fmoc chemistry. The ultimate synthetic step involves oxidation of the furanol functionality to the corresponding furanone using an oxidant such as Dess-Martin periodinane. Alternatively, the oxidation may be carried out prior to subsequent modifications at the N-terminus.

Many R3 groups are accessed from commercially available amino acid residues such as L-leucine, L-norleucine, L-phenylalanine etc. Other branched and unsaturated amino acid building blocks are as shown in Medivir UK's PCT/GB01/02162 claiming priority from British patent application GB 00025386-4 filed 17 May 2000 the contents of which are incorporated by reference.

Access to sulphonyl bearing C1-C7alkyl or ArC1-C7alkyl R3 groups, for instance arylalkylC0-2sulphonylmethyl functionalities can come from the suitably protected amino acid cysteine. Mitsunobu coupling of the cysteinyl thiol with aryl alcohols such as phenol yield the protected amino acid containing the phenylthiomethyl R3 sidechain that is readily oxidised using m-chloroperbenzoic acid to provide the R3 sidechain phenylsulphonylmethyl. The benzylsulphonylmethyl and phenethylsulphonylmethyl R3 sidechain containing amino acids can be prepared by nucleophilic substitution of the cysteinyl thiol with benzyl bromide and phenethyl bromide respectively.

Oxidation of the resulting sulphides with m-chloroperbenzoic acid provides the suitably protected amino acids with the benzylsulphonylmethyl and phenethylsulphonylmethyl R3 sidechain.

### DETAILED DESCRIPTION OF THE EMBODIMENTS

### Solution Phase Chemistry

### EXAMPLE 1

### Following General Chemistry Scheme 14

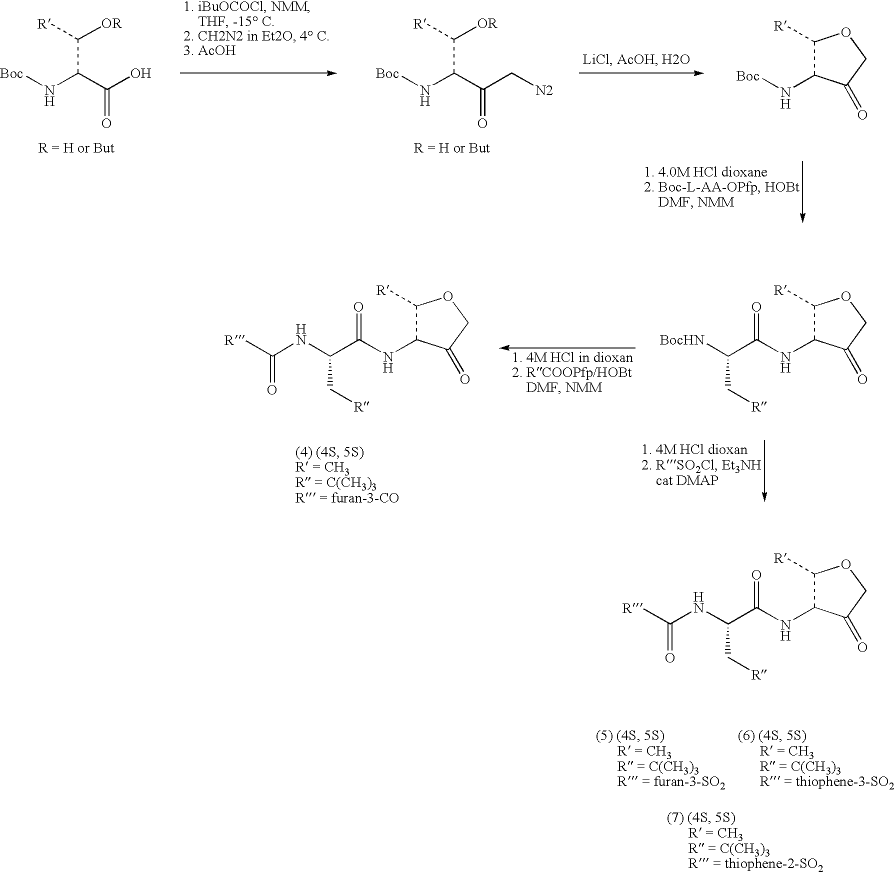

### (a) General Method for the Synthesis of N-Boc Protected Diazoketones, Exemplified by (2S,3S)-N-Boc-O-t-butyl-L-threonyldiazomethane (1)

(2S,3S)-N-Boc-O-t-butyl-L-threonine (1.2 g, 4.2 mmol) was dissolved in dry DCM (20 mL) and N-methylmorpholine (1 mL, 2.2 eq) added. The reaction mixture was cooled to −15° C. and stirred under an atmosphere of argon. Isobutyl chloroformate (0.56 mL, 4.3 mmol) was added and the mixture stirred for 10 mins at −15° C. A solution of diazomethane in diethyl ether (45 mL, approx 40 mmol) was added and the reaction allowed to warm to room temperature over 1 hr, then acetic acid was added dropwise until effervescence had ceased. The reaction mixture was diluted with DCM (100 mL) and washed successively with saturated aqueous sodium bicarbonate (2×75 mL), water (75 mL) and brine (75 mL) and dried over sodium sulphate. The solvent was removed in vacuo to give crude (2S,3S)-N-Boc-O-t-butyl-L-threonyidiazomethane (1.2 g, ˜100%) as a pale yellow oil. The above synthesis was repeated 9 times and the total crude product pooled (12 g) and used without purification for the next stage.

### (b) General Method for the Synthesis of Boc-3(2H)-furanones, Exemplified by dihydro-(4S-amino-[N-Boc])-5S-methyl-3(2H)-furanone (2)

A solution of lithium chloride (13.6 g, 320 mmol) in 80% aqueous acetic acid (400 mL) was cooled to 5° C. and added to crude (2S,3S)-N-Boc-O-t-butyl-L-threonyldiazomethane (1) (9.6 g) with stirring. The oil dissolved over 10 mins and stirring continued for a further 1 hr slowly warming to room temperature, with evolution of gas. The solvents were removed in vacuo and the residue taken into EtOAc (250 mL) and washed successively with water (250 mL), saturated aqueous sodium bicarbonate (2×100 mL) and brine (75 mL), then dried over sodium sulphate. The solvent was removed in vacuo and the crude product purified by flash chromatography over silica gel (150 g) eluting with EtOAc/heptane (1:2, v/v). Two fractions were pooled and the quicker eluting fraction reduced in vacuo to approx 50 mL heptane and left to crystallise to give dihydro-(4S-amino-[N-Boc])-5S-methyl-3(2H)-furanone (2) as a white solid, yield 4.05 g, 18.8 mmol, 58%. Electrospray-MS m/z 216 (MH⁺), 160 (MH⁺−56), elemental analysis C₁₀H₁₇O₄N (req) % C 55.80, % H 7.96, % N 6.51, (fnd) % C 55.82, % H 7.86, % N 6.44.

δ_{H}(⁵⁰⁰ MHz; CDCl₃); 1.41 (9H, s, C(CH₃)₃), 1.49 (3H, d, J 6, 5S—CH₃), 3.72 (1H, bm, furanone CHα), 3.90-4.02 (2H, 5S—H+1×furanone COCH₂O), 4.22 (1H, d, J 17.4, 1×furanone COCH₂O), 4.85 (1H, bs, furanone, NH). δ_{C} (125 MHz; CDCl₃); 19.34 (5S—CH₃), 28.45 (C(CH₃)₃), 62.79 (furanone CHα), 71.06 (furanone COCH₂O), 77.96 (5S—CHCH₃), 80.88 (C(CH₃)₃), 155.6 ((CH₃)₃CO—CO), 212.6 (furanone CO).

### (c) General Method for N-Terminal Extension, Exemplified by dihydro-(4S-amino-[N-Boc-L-tert-butylalanyl])-5S-methyl-3(2H )-furanone (3)

Dihydro-(4S-amino-[N-Boc])-5S-methyl-3(2H)-furanone (2) (1.0 g, 4.6 mmol) was treated with a solution of 4.0 M HCl in dioxan (25 mL) at room temperature for 1 hr. The solvents were removed in vacuo and the residue azeotroped with 2× toluene to give the hydrochloride salt as a white solid. Boc-L-tert-butylalanine pentafluorophenyl ester (2.0 g, 1.05 eq) and 1-hydroxybenzotriazole hydrate (0.735 g, 1.05 eq) were dissolved in DMF (20 mL) and after 5 mins added to the above salt. The clear solution was then treated with N-methylmorpholine (0.51 g, 0.56 mL, 1.1 eq) and left at room temperature for 2 hrs. The solvents were removed in vacuo and the crude product purified by flash chromatography over silica gel (50 g) eluting with EtOAc/heptane (1:3, v/v), then EtOAc/heptane (1:2, v/v). Fractions were pooled and reduced in vacuo to give dihydro-(4S-amino-[N-Boc-L-tert-butylalanyl])-5S-methyl-3(2H)-furanone (3) as a white solid, yield 1.31 g, 3.82 mmol, 83%.

Electrospray-MS m/z 343 (MH⁺), 287 (MH⁺−56).

This methodology is readily applicable to the corresponding N-Boc-protected pyranone or (1,5-anhydro-3-[(tert-butoxycarbonyl)amino]-3,4-dideoxy-4-ethyl-D-xylitol

### (d) General Method for Addition of Capping Group, Exemplified by benzofuran-2-carboxylic acid [3,3-dimethyl-1S-(2S-methyl-4-oxo-tetrahydrofuran-3S-ylcarbamoyl)butyl]amide (4)

Dihydro-(4S-amino-[N-Boc-L-tert-butylalanyl])-5S-methyl-3(2H)-furanone (3) (1.03 g, 3.0 mmol)) was treated with a solution of 4.0M HCl in dioxan (25 mL) at room temperature for 1 hr. The solvents were removed in vacuo and the residue azeotroped with 2× toluene to give the hydrochloride salt as a white solid.

Benzofuran-2-carboxypentafluorophenyl ester (1.05eq) and 1-hydroxybenzotriazole hydrate (1.05 eq) are dissolved in DMF (15 mL) and after 5 mins added to the above salt. The clear solution was then treated with N-methylmorpholine (1.1 eq) and left at room temperature for 2 hrs. The solvents are removed in vacuo and the crude product purified by flash chromatography over silica gel (50 g) eluting with EtOAc/heptane (3:2, v/v). Fractions are pooled and reduced in vacuo to give the title compound.

### EXAMPLE 2

### 4,4-Dimethyl-2S-(benzofuran-2-sulfonylamino)pentanoic acid (2S-methyl-4-oxo-tetrahydrofuran-3S-yl)amide (5)

### (a) General Method for Addition of Sulphonyl Capping Group, Exemplified by 4,4-Dimethyl-2S-(benzofuran-2-sulfonylamino)pentanoic acid (2S-methyl-4-oxo-tetrahydrofuran-3S-yl)amide (5)

Dihydro-(4S-amino-[N-Boc-L-tert-butylalanyl])-5S-methyl-3(2H)-furanone (3) (34 mg, 0.1 mmol) was treated with a solution of 4.0M HCl in dioxan (5 mL) at room temperature for 1 hr. The solvents were removed in vacuo and the residue azeotroped with 2× toluene to give the hydrochloride salt as a white solid.

Hydrochloride salt was dissolved in dry DCM (2 mL) and benzofuran-2-sulphonylchloride added followed by diisopropylethylamine (3 eq) and catalytic N,N-dimethylaminopyridine (2 mg). After 2 hr at room temperature, the solution was diluted with DCM (15 mL) and washed successively with 0.1N HCl (25 mL), water (2×25 mL) and brine (25 mL), then dried over sodium sulphate. The solvent was removed in vacuo and the crude product purified by flash chromatography over silica gel (15 g) eluting with EtOAc/heptane (1:1, v/v). Fractions were pooled and reduced in vacuo to give the title compound.

### General Synthesis of Chiral β-alkyl serine aminoacids

Adapted from Blaskovich, M. A., Evinder, G., Rose, N. G. W., Wilkinson, S., Luo, Y. and Lajoie, G. A. J. Org. Chem, 63, 3631-3646, 1998. (Following scheme 2).

### EXAMPLE 5

### (2S,3S)β-hydroxynorvaline (15)

### (a) N-Benzyloxycarbonyl-L-serine 3-methyl-3-(hydroxymethyl)oxetane ester (8)

N-Cbz-L-serine (10 g, 41.8 mmol) was dissolved in DCM (450 mL) and DMF (14 mL) and added dropwise over 2.5 h to a stirred solution of WSC. HCl (12 g, 62.7 mmol), N′N-dimethylaminopyridine (260 mg, 2.1 mmol) and 3-methyl-3-oxetane methanol (84 mL, 0.84 mmol) cooled to 0° C. The reaction was warmed to room temperature and allowed to stir overnight. The mixture was washed with 0.1M HCl (200 mL), water (200 mL), 10% Na₂CO₃ (200 mL×2) and water (200 mL×2), dried (Na₂SO₄) and the solvent evaporated in vacuo to afford a pale yellow oil. Purification by column chromatography (4:1, EtOAc:heptane) and subsequent recrystallisation (1:1, EtOAc:heptane) yielded the target intermediate as a white crystalline solid, 8.07 g, 60%; TLC (4:1, EtOAc:heptane), Rf=0.28, electrospray-MS m/z 324.1 (MH⁺).

δ_{□} (400 MHz; CDCl₃) 1.28 (3H, s, CH₃), 3.04 (1H, t, J 6.2, CHNH), 3.90-3.91 (1H, br m, OH), 4.10-4.13 (2H, m, CH₂OH), 4.41-4.55 (6H, m, 3×CH₂), 5.13 (2H, s, OCH₂), 5.82 (1H, d, J 7.7, NH), 7.35-7.36 (5H, m, C₆H₅). δ_{C} (100 MHz; CDCl₃)□ 20.75 (CH₃), 39.67 (CH₂OH), 56.39 (CHNH), 63.37 (CH₂), 67.19 (CH₂), 68.94 (CH₂), 79.50 (OCH₂), 128.16 (C₆H₅), 128.27 (C₆H₅), 128.58 (C₆H₅), 136.14 (C₆H₅), 156.25 (CO₂NH), 170.74 (CO₂).

### (b) 1-[N-Benzyloxycarbonyl-(1S)-1-amino-2-hydroxyethyl]-4-methyl-2,6,7-trioxabicyclo[2.2.2]oxetane (9)

Compound (8) (10.23 g, 28.6 mmol) was dissolved in anhydrous DCM (150 mL) and cooled to 0° C. under N₂. A solution of boron trifluoride etherate (0.10 mL, 0.77 mmol) in anhydrous DCM (10 mL) was added and the mixture stirred for 30 minutes at 0° C., then at room temperature overnight. Triethylamine (1.2 mL, 8.30 mmol) was added and the reaction mixture stirred for 30 minutes before being concentrated to a thick colourless oil. Purification by column chromatography (4:1, EtOAc:heptane) and subsequent recrystallisation (1:1, EtOAc:heptane) yielded (9) as a white crystalline solid, 8.06 g, 80%; TLC (4:1, EtOAc:heptane) Rf=0.27, electrospray-MS m/z 324.1 (MH⁺).

δ_{□} (400 MHz; CDCl₃)□ 0.78 (3H, s, CH₃), 2.67 (1H, m, CHNH), 3.64-3.69 (1H, m, CH₂OH), 3.80-3.83 (1H, m, CH₂OH), 3.88 (6H, s, CH₂×3), 5.09 (2H, dd, J 18.9, 12.3, OCH₂), 5.38 (1H, d, J 8.7, NH), 7.26-7.34 (5H, m, C₆H₅): δ_{C} (100 MHz; CDCl₃)□ 14.11 (CH₃), 30.39 (CCH₃), 55.26 (CHNH), 61.75 (CH₂OH), 66.76 (CH₂O), 72.53 (CH₂×3), 108.29 (CO₃), 127.98 (C₆H₅), 128.32 (C₆H₅), 136.31 (C₆H₅), 156.31 (CO₂NH).

### (c) 1-[N-Benzyloxycarbonyl-(1S)-1-amino-2-oxoethyl]-4-methyl-2,6,7-trioxabicyclo[2.2.2]oxetane (10)

Compound (9) (6.45 g, 20.0 mmol) was dissolved in anhydrous DCM (55 mL) under N₂ and cooled to −78° C. in flask 1. Oxalyl chloride (2.8 mL, 31.9 mmol) was added to anhydrous DCM (85 mL) in a separate flask (flask 2) under N₂ and cooled to −78° C. Anhydrous dimethylsulphoxide (4.7 mL, 65.8 mmol) was added to the oxalyl chloride solution and the mixture stirred at −78° C. for 15 minutes. The alcohol solution was transferred over 20 minutes by cannula to flask 2 and rinsed with anhydrous DCM (35 mL). The resulting cloudy, white mixture was stirred for 1.5 hours at −78° C.

Diisopropylethylamine (17.4 mL, 99.7 mmol) was added and the solution stirred for 30 minutes at −78° C. and 10 minutes at 0° C. Ice-cold DCM (140 mL) was added and the solution washed with ice-cold NH₄Cl (20% saturated solution; 3×140 mL) and saturated NaCl (140 mL), dried (MgSO₄) and the solvent evaporated in vacuo to afford a yellow solid (10), 5.08 g, 79%; TLC (3:1, EtOAc:heptane), Rf=0.56, electrospray-MS m/z 322.1 (40%) (MH⁺), 340.2 (100%) (MH⁺+H₂O).

δ_{H} (400 MHz; CDCl₃); 0.82 (3H, s, CH₃), 3.93 (6H, s, CH₂×3), 4.60 (1H, d, J 8.8, CHNH), 5.12 (2H, dd, J 14.9, 12.4, OCH₂), 5.35 (1H, br d, J 8.0, NH), 7.30-7.36 (5H, m, C₆H₅), 9.68 (1H, s, HCO). δ_{C} (100 MHz; CDCl₃)□ 14.25 (CH₃), 30.86 (CCH₃), 63.25 (CHNH), 67.22 (CH₂O), 72.88 (CH₂×3), 107.16 (CO₃), 128.13 (C₆H₅), 128.46 (C₆H₅), 136.13 (C₆H₅), 156.17 (CO₂NH), 195.66 (CHO).

### (d) 1-[N-(Benzyloxycarbonyl)-(1S,2R)-1-amino-2-hydroxybutyl]-4-methyl-2,6,7-trioxabicyclo[2.2.2]oxetane (11)

Compound (10) (2 g, 5.73 mmol) was dissolved in anhydrous DCM:Et₂O (1:1) under N₂. A solution of EtMgBr (3M solution in Et₂O; 7.6 mL, 22.9 mmol) was added quickly at −78° C. and stirred vigorously. After 30 minutes the reaction was quenched by pouring into 5% NH₄Cl (500 mL). DCM (500 mL) was added, the organic layer separated and washed with 3% NH₄Cl (500 mL) and brine (500 mL), dried (Na₂SO₄) and the solvent evaporated in vacuo to afford a yellow oil. Purification by column chromatography (1:10, EtOAc:DCM) and subsequent recrystallisation (EtOAc:heptane) yielded a white crystalline solid (11), 1.32 g, 60%; TLC (1:10, EtOAc:DCM) Rf=0.25, electrospray-MS m/z 352.2 (20%) (MH⁺), 370.3 (100%) (MH⁺+H₂O).

δ_{□} (400 MHz; CDCl₃)□ 0.82 (3H, s, CH₃), 0.94 (3H, t, J 7.4, CH₂CH₃), 1.36-1.53 (2H, m, CH₂CH₃), 3.85 (1H, d, J 10.3, CH), 3.93 (6H, s, CH₂×3), 4.05 (1H, t, J 6.8, CH), 5.13-5.14 (2H, dd, J 16.4, 12.7, OCH₂), 5.32 (1H, d, J 10.2, NH), 7.30-7.36 (5H, m, C₆H₅). δ_{C} (100 MHz; CDCl₃) [symbol] 10.11 (CH₂CH₃), 14.34 (CH₃), 25.98 (CH₂CH₃), 30.65 (CCH₃), 56.05 (CHOH), 66.83 (CH₂O), 70.81 (CHNH), 72.76 (CH₂×3), 108.93 (CO₃), 127.65 (C₆H₅), 128.45 (C₆H₅), 136.65 (C₆H₅), 156.83 (CO₂NH).

### (e) 1-[N-Benzyloxycarbonyl-(1S)-1-amino-2-oxobutyl]-4-methyl-2,6,7-trioxabicyclo[2.2.2]oxetane (12)

Compound (11) (1.32 g, 3.8 mmol) was dissolved in anhydrous DCM (10 mL) under N₂ and cooled to −78° C. in flask 1. Oxalyl chloride (2M solution in DCM; 3 mL, 6.0 mmol) was diluted with anhydrous DCM (10 mL) in a separate flask (flask 2) under N₂ and cooled to −78° C. Anhydrous dimethylsulphoxide (0.88 mL, 12.4 mmol) was added to the oxalyl chloride solution and the mixture stirred at −78° C. for 15 minutes. The alcohol solution was transferred over 20 minutes by cannula to flask 2 and rinsed with anhydrous DCM (10 mL). The resulting cloudy, white mixture was stirred for 2 hr 15 min at −78° C. DIPEA (3.3 mL, 18.8 mmol) was added and the solution stirred for 30 minutes at −78° C. and 10 minutes at 0° C. Ice-cold DCM (25 mL) was added and the solution washed with ice-cold NH₄Cl (5% saturated solution; 3×25 mL) and saturated NaCl (25 mL), dried (Na₂SO₄) and the solvent evaporated in vacuo to afford an orange oil. Purification by column chromatography (2:3, EtOAc:heptane) yielded a colourless oil (12), 556 mg, 45%; TLC (2:3, EtOAc:heptane) Rf=0.25, electrospray-MS m/z 350.2 (60%) (MH⁺), 368.2 (100%) (MH⁺+H₂O).

δ_{□} (400 MHz; CDCl₃)□ 0.80 (3H, s, CH₃), 1.06 (3H, t, J 7.2, CH₂CH₃), 2.48-2.56 (1H, m, CHCH₃), 2.80-2.88 (1H, m, CHCH₃), 3.90 (6H, s, CH₂×3), 4.60 (1H, d, J 8.8, CHNH), 5.09 (2H, s, OCH₂), 5.66 (1H, d, J 8.5, NH), 7.30-7.35 (5H, m, C₆H₅). δ_{C} (100 MHz; CDCl₃)□ 7.53 (CH₂CH₃), 14.25 (CH₃), 30.57 (CCH₃), 35.74 (CH₂CH₃), 62.33 (CHNH), 67.05 (CH₂O), 72.94 (CH₂×3), 106.98 (CO₃), 128.10 (C₆H₅), 128.46 (C₆H₅), 136.31 (C₆H₅), 155.99(CO₂NH).

### (f) 1-[N-(Benzyloxycarbonyl)-(1S,2S)-1-amino-2-hydroxybutyl]-4-methyl-2,6,7-trioxabicyclo[2.2.2]oxetane (13)

Compound (12) (2.77 g, 7.9 mmol) and LiBH₄ (1.73 g, 79 mmol) were cooled to −78° C. under N₂. A solution of DCM:CH₃OH (1.5:1; 332 mL cooled to −78° C.) was added and the solution stirred at −78° C. overnight. After being warmed to room temperature, the solution was poured into 5% NH₄Cl solution (500 mL) and DCM (300 mL) added. The organic layer was separated, washed with 5% NH₄Cl solution (500 mL) and brine (400 mL), dried (Na₂SO₄) and the solvent evaporated in vacuo to afford a white solid (13), 2.51 g, 90%; TLC (1:1, EtOAc:heptane) Rf=0.23, electrospray-MS m/z 352.2 (40%) (MH⁺), 370.3 (100%) (MH⁺+H₂O).

δ_{□} (400 MHz; CDCl₃)□ 0.82 (3H, s, CH₃), 0.97 (3H, t, J 7.4, CH₂CH₃), 1.44-1.45 (1H, m, CHCH₃), 1.63-1.68 (1H, m, CHCH₃), 3.44 (1H, d, J 4.0, CHOH), 3.66-3.69 (1H, m, CHNH), 3.92 (6H, s, CH₂×3), 5.04 (1H, d, J 9.8, NH), 5.16 (2H, d, J 6.1, OCH₂), 7.36 (5H, d, J 4.3, C₆H₅), δ_{C} (100 MHz; CDCl₃)□ 9.79 (CH₂CH₃), 14.27 (CH₃), 26.10 (CH₂CH₃), 30.56 (CCH₃), 57.57 (CHOH), 66.94 (CH₂O), 69.80 (CHNH), 72.66 (CH₂×3), 108.89 (CO₃), 128.06 (C₆H₅), 128.47 (C₆H₅), 136.51 (C₆H₅), 156.49 (CO₂NH).

### (g) (1S,2S)-(1-amino-2-hydroxybutyl)-4-methyl-2,6,7-trioxabicyclo[2.2.2]oxetane (14)

Compound (13) (2.51 g, 7.1 mmol) was dissolved in ethanol (220 mL) and 10% Pd/C (218 mg) added. The reaction mixture was stirred overnight in the presence of H₂. The catalyst was removed by filtration through celite and the solvent evaporated in vacuo to afford a thick oil. Purification by column chromatography (20:1, DCM:MeOH) yielded a pale yellow oil (14), 1.24 g, 92%, which crystallised on standing; TLC (5:1, DCM:MeOH) Rf=0.51, electrospray-MS m/z 218.1 (MH⁺).

□_{□} (400 MHz; CDCl₃)□ 0.83 (3H, s, CH₃), 0.98 (3H, t, J 7.4, CH₂CH₃), 1.38-1.48 (1H, m, CHCH₃), 1.71-1.78 (1H, m, CHCH₃), 2.77 (1H, d, J 7.1, CHNH₂), 3.62-3.66 (1H, m, CHOH), 3.93 (6H, s, CH₂×3). □_{C} (100 MHz; CDCl₃)□ 9.57 (CH₂CH₃), 14.37 (CH₃), 26.01 (CH₂CH₃), 30.52 (CCH₃), 58.52 (CHOH), 72.59 (CHNH₂), 72.67 (CH₂×3), 109.62 (CO₃).

### (h) (2S,3S)β-hydroxynorvaline (15)

Compound (14) (1.24 g, 5.5 mmol) was dissolved in DCM (68 mL) and trifluoroacetic acid (1.58 mL) and H₂O (1.13 mL) added. The resulting cloudy, white solution was stirred at room temperature for 30 minutes and the solvent evaporated in vacuo. The colourless residue was dissolved in MeOH (66 mL) and H₂O (17 mL) and 10% Cs₂CO₃ (9.2 g in 92 mL H₂O) added. After stirring overnight at room temperature, the solution was acidified with 2 M HCl (˜35 mL) to pH<3. The solution was loaded onto a cation exchange column (Bio-Rad AG 50W-X8 100-200 mesh, hydrogen form, 4.5×20 cm) washed with 0.01 M HCl (500 mL) and H₂O (500 mL) and eluted with 2M NH₄OH (2 L) then lyopholised to afford a pale yellow solid. The solid was washed with MeOH to yield an off-white solid (15), 227 mg, 30%; TLC (4:1:1, butan-2-ol:AcOH:H₂O) Rf=0.26, electrospray-MS m/z 134.1 (MH⁺), elemental analysis C₅H₁₁O₃N (req) % C 45.10, % H 8.33, % N 10.52, (fnd) % C 44.67, % H 8.03, % N 9.92.

δ_{□} (400 MHz; CDCl₃)□ 92:8 erythro (2S, 3S): threo (2S,3R), 1.00 (3H, t, J 7.4, CH₃), 1.40-1.54 (2H, m, CH₂), 3.41 (0.08H, d, J 4.2, CH), 3.61 (0.92H, d, J 4.2, CH), 3.60-3.65 (0.08H, m, CH), 3.66-3.69 (0.92H, m, CH). δ_{C} (100 MHz; CDCl₃)□ 11.02 (CH₂CH₃), 25.26 (CH₂CH₃), 61.26 (CHOH), 72.09 (CHNH₂), 172.03 (CO₂H).

### General Method for the Synthesis of Fmoc-3(2H)-furanones

Exemplified by dihydro-(4S-amino-[N-Fmoc])-5S-ethyl-3(2H)-furanone (18), following the general chemistry detailed in scheme 1.

### (a) Preparation of Fmoc-(2S,3S)-β-ethylserine (16)

(2S,3S)□-hydroxynorvaline (15) (277 mg, 2.07 mmol) and sodium carbonate (2.1 eq, 460 mg) were dissolved with stirring and ice-cooling in water (25 mL) and THF (10 mL). 9-Fluorenylmethyl chloroformate (1.05 eq, 560 mg) in THF (15 mL) was added over 45 mins and the mixture stirred for a further 1 hr at room temperature. Chloroform (100 mL) and water (50 mL) were added and the mixture acidified to pH2 with 0.1 N HCl. The organic layer was collected and the aqueous washed with a further 2×100 mL chloroform. The combined organics were backwashed with brine (1×300 mL) and dried over magnesium sulphate. The chloroform was reduced in vacuo to yield a fine white solid. The solid was dissolved in tert-butyl methylether (25 mL) with heating and heptane (75 mL) added to give a cloudy solution. The mixture was cooled to −20° C. and each 30 mins further heptane (75 mL) added for 4 cycles. The precipitate was filtered off and dried in vacuo to a fine white solid (16) 590 mg, 80.6%; TLC (CHCl₃; MeOH 3:1) Rf=0.40, electrospray-MS m/z 356.2 (MH⁺).

### (b) Preparation of (2S, 3S)-N-Fmoc-β-ethylserinydiazomethane (17)

Following the general method detailed in example 1.(a) for compound (1), Fmoc-(2S,3S)-ethylserine (16) (560 mg) was converted to a yellow solid (600 mg) (17) used without purification.

### (c) Preparation of dihydro-(4S-amino-[N-Fmoc])-5S-ethyl-3(2H)-furanone (18)

A solution of lithium chloride (1.0 g, 23.5 mmol) in 80% aqueous acetic acid (10 mL) was cooled to 5° C. and added to crude (2S,3S)-N-Fmoc-□-ethylserinydiazomethane (17) (0.6 g) with stirring. The oil dissolved over 10 mins and stirring continued for a further 1 hr slowly warming to room temperature, with evolution of gas. The solvents were removed in vacuo and the residue taken into EtOAc (50 mL) and washed successively with water (50 mL), saturated aqueous sodium bicarbonate (2×100 mL) and brine (75 mL), then dried over sodium sulphate. The solvent was removed in vacuo and the crude product purified by flash chromatography over silica gel (25 g) eluting with EtOAc/heptane (1:3, v/v). Desired fractions were pooled and reduced in vacuo to give dihydro-(4S-amino-[N-Fmoc])-5S-ethyl-3(2H)-furanone (18) as a white solid, yield 320 mg, 0.91 mmol, 58%. Electrospray-MS m/z 352 (MH⁺), HRMS C₂₁H₂₁O₄NNa requires M, 374.1368, found: MNa⁺, 374.1368. ( [symbol] −1.49 ppm), analytical HPLC Rt=13.61 mins (98.4%), elemental analysis C₂₁H₂₁O₄N (req) % C 71.78, % H 6.02, % N 3.99, (fnd) % C 70.95, % H 6.22, % N 3.81.

δ_{□} (500 MHz; CDCl₃); 1.05 (3H, m, CH₂CH₃), 1.76, 1.94 (2H, bm, CH₂CH₃), 3.83 (1H, bm, furanone CHβ), 3.88 (1H, bm, furanone CHα), 4.02 (1H, d, J 17.3, 1×furanone COCH₂O), 4.23 (2H, m, 1×furanone COCH₂O+Fmoc CHCH₂O), 4.42 (2H, b, Fmoc CHCH₂O), 5.05 (1H, b, furanone, NH), 7.35 (2H, t, J 7.4, Fmoc aromatic), 7.42 (2H, t, J 7.3, Fmoc aromatic), 7.58 (2H, t, J 7.4, Fmoc aromatic), 7.77 (2H, t, J 7.4, Fmoc aromatic).

δ_{C} (125 MHz; CDCl₃); 8.90 (5S—CH₂CH₃), 26.14 (5S—CH₂CH₃), 46.90 (Fmoc CHCH₂O), 60.50 (furanone CHα), 66.99 (Fmoc CHCH₂O), 70.43 (furanone COCH₂O), 81.65 (furanone CHβ), 119.76 (Fmoc aromatic), 124.72 (Fmoc aromatic), 126.85 (Fmoc aromatic), 127.53 (Fmoc aromatic), 141.09 (Fmoc aromatic), 143.37 (Fmoc aromatic), 155.76 (OCONH), 211.72 (furanone CO).

### (d) Preparation of (2S,3R)-N-Fmoc-O-t-butyl-L-threonyldiazomethane (19)

Following the general method detailed in example 1. (a) for compound (1), Fmoc-(2S,3R)-O-t-butyl-L-threonine (1.99 g, 5 mmol) was converted to (2S,3R)-N-Fmoc-O-t-butyl-L-threonyldiazomethane (19) (2.11 g, 100%) as a pale yellow immobile oil. This compound was carried through to the next stage without further purification. Electrospray-MS m/z 444 (MNa⁺, 20%), 394 (MH⁺—N₂, 70%) and 338 (MH⁺-tbutyl-N₂, 100%).

### (e) Preparation of (2R,3S)-N-Fmoc-O-t-butyl-D-threonyldiazomethane (20)

Following the general method detailed in example 1. (a) for compound (1), Fmoc-(2R,3S)-O-t-butyl-D-threonine (0.4 g, 1 mmol) was converted to (2S,3R)-N-Fmoc-O-t-butyl-L-threonyidiazomethane (20) (0.48 g, 111%) as a pale yellow immobile oil. This compound was carried through to the next stage without further purification. Electrospray-MS m/z 394 (MH⁺—N₂, 60%) and 338 (MH⁺-tbutyl-N₂, 100%).

### (f) Preparation of (2S,3S)-N-Fmoc-O-t-butyl-L-allo-threonyldiazomethane (21)

Following the general method detailed in example 1.(a) for compound (1), Fmoc-(2S,3S)-O-t-butyl-L-allo-threonine (0.4 g, 1 mmol) was converted to (2S,3S)-N-Fmoc-O-t-butyl-L-allo-threonyldiazomethane (21) (0.53 g, 123%) as a pale yellow immobile oil. Electrospray-MS m/z 394 (MH⁺—N₂, 90%) and 338 (MH⁺-tbutyl-N₂, 60%).

### (i) Preparation of dihydro-(4R-amino-[N-Fmoc])-5R-methyl-3(2H)-furanone (22)

Following the general method detailed for cyclisation of (17) to (18), diazoketone (19) cyclised to give dihydro-(4R-amino-[N-Fmoc])-5R-methyl-3(2H)-furanone (22) isolated as a white solid, yield 69%, electrospray-MS m/z 338 (MH⁺, 100%), analytical HPLC Rt=14.59 mins (97.7%).

δ_{□} (500 MHz; CDCl₃); 1.50 (3H, brd, CH₃), 3.80 (1H, brt, furanone CHα), 3.97 (1H, brm, furanone CH□), 3.99 (1H, d, J 17.7, 1×furanone COCH₂O), 4.22 (1H, t, J 6.7, Fmoc CHCH₂O), 4.25 (1H, d, J 17.7, 1×furanone COCH₂O), 4.44 (2H, b, Fmoc CHCH₂O), 5.11 (1H, b, NH), 7.32 (2H, t, J 7.4, Fmoc aromatic), 7.41 (2H, t, J 7.4, Fmoc aromatic), 7.58 (2H, t, J 7.4, Fmoc aromatic), 7.76 (2H, t, J 7.4, Fmoc aromatic). δ_{C} (125 MHz; CDCl₃); 19.1 (CH₃), 47.2 (Fmoc CHCH₂O), 62.7 (furanone CHα), 67.3 (Fmoc CHCH₂O), 70.8 (furanone COCH₂O), 77.4 (furanone CHβ), 120.1 (Fmoc aromatic), 125.0 (Fmoc aromatic), 127.1 (Fmoc aromatic), 127.8 (Fmoc aromatic), 141.4 (Fmoc aromatic), 143.7 (Fmoc aromatic), 156.1 (OCONH), 211.8 (furanone CO).

### (j) Preparation of dihydro-(4S-amino-[N-Fmoc])-5S-methyl-3(2H)-furanone (23)

Following the general method detailed for cyclisation of (17) to (18), diazoketone (20) cyclised to give dihydro-(4S-amino-[N-Fmoc])-5S-methyl-3(2H)-furanone (23) isolated as a white solid, yield 70%, electrospray-MS m/z 338 (MH⁺, 75%), analytical HPLC Rt=14.62 mins (98.9%).

### (k) Preparation of dihydro-(4S-amino-[N-Fmoc])-5S-methyl-3(2H)-furanone (23)

Following the general method detailed for cyclisation of (17) to (18), diazoketone (21) cyclised to give dihydro-(4R-amino-[N-Fmoc])-5R-methyl-3(2H)-furanone (23) isolated as a white solid, yield 64%, electrospray-MS m/z 338 (MH⁺, 100%), analytical HPLC Rt=14.68 mins (97.5%).

### General Method for Preparation of N-protected-4-aminopyranol Building Blocks

This route is exemplified by the 4S 5S enantiomer, but is applicable to other configurations as discussed above.

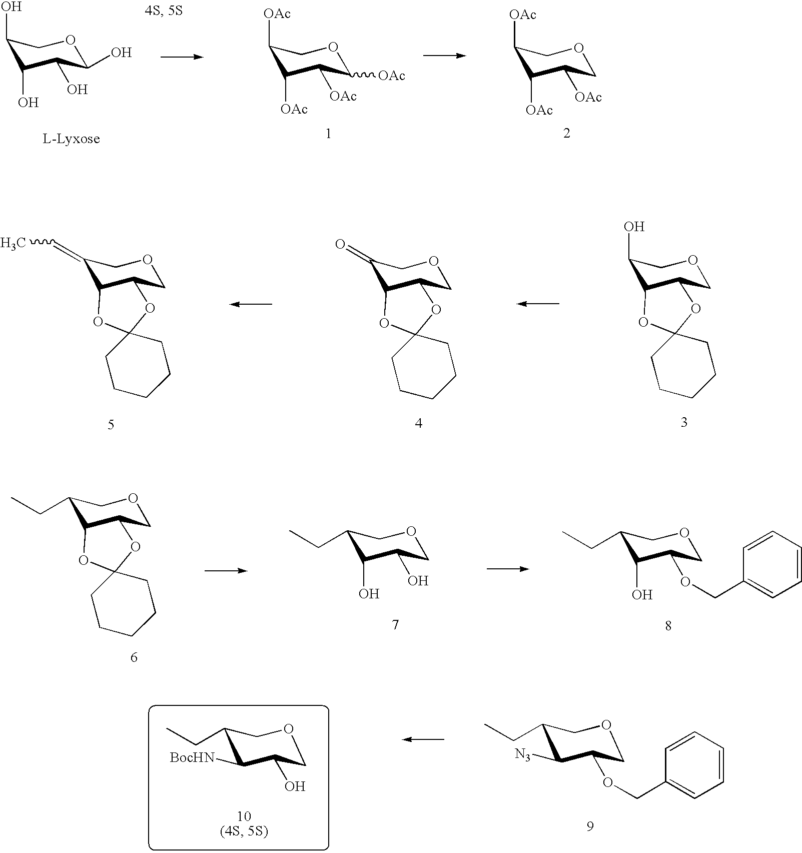

### 1,2,3,4-Tetra-O-acetyl-L-lyxopyroanose (1)

L-Lyxopyroanose (25.0 g, 166 mmol) was dissolved in pyridine (150 ml) and cooled on an icebath, acetic anhydride (75 ml) was added and the solution was stirred at room temperature. After 2 hours tic (pentane:ethyl acetate 1:1) indicated complete conversion of the starting material into a higher migrating spot. The solution was concentrated and co-evaporated three times with toluene which gave a pale yellow syrup.

NMR data 400 MHz (CDCl₃): ¹H, δ 2.06 (s, 3H), 2.08 (s, 3H), 2.14 (s, 3H), 2.16 (s, 3H), 3.71 (dd, 1H), 4.01 (dd, J=5.0, 11.7 Hz, 1H), 5.17-5.26 (m, 2H), 5.37 (dd, J=3.5, 8.8 Hz, 1H), 6.0 (d, J=3.2 Hz, 1H). ¹³C, δ 20.9, 20.9, 21.0, 21.0, 62.2, 66.7, 68.4, 68.4, 90.8, 168.8, 169.9, 170.0, 170.1.

### 2,3,4-Tri-O-acetyl-1,5-anhydro-L-arabinitol (2)

Trimethylsilyl trifluormethanesulphonate (60 ml, 333 mmol) was added to a solution of crude 1,2,3,4-tetra-O-acetyl-L-lyxopyroanose constituting the yiled from the step above in acetonitrile (200 ml), the solution was cooled on an ice bath and triethylsilane (80 ml, 500 mmol) was added dropwise. The solution was stirred at room temperature and the reaction was monitored by GC. When the reaction was completed (after 3 hours) the solution was neutralised with sodium hydrogen carbonate (s), diluted with dichloromethane and washed with water. The organic phase was dried with magnesium sulphate, filtered and concentrated. The obtained oil was purified by silica gel flash column chromatography (pentane:ethyl acetate 5:1, 4:1, 3:1) which gave 32 g, 74% (from free lyxose) of the reduced compound.

NMR data 400 MHz (CDCl₃): ¹H, δ 2.06 (s, 3H), 2.07 (s, 3H), 2.11 (s, 3H), 3.36-3.41 (m, 1H), 3.64 (dd, J=2.4, 12.2 Hz, 1H), 3.87 (m, 1H), 4.03 (m, 1H), 5.10-5.15(m, 2H), 5.28-5.31 (m, 1H).

### 1,5-Anhydro-3,4-O-cyclohexylidene-L-arabinitol (3)

A solution of 1-deoxy-2,3,4-tri-O-acetyl-L-lyxopyroanose (20.8 g, 80 mmol) in methanol (125 ml) was treated with a catalytic amount of 1M methanolic sodium methoxide. After stirring for 1 hour at room temperature tic (ethyl acetate:methanol 3:1) indicated complete conversion into a lower migrating spot. The solution was neutralised with Dowex H⁺, filtered and concentrated, which gave a colourless oil.

The oil was suspended in dichloromethane (70 ml) and cyclohexanone diethyl ketal (41 g, 240 mmol) was added followed by p.toluenesulphonic acid until acidic pH. After a few minutes the suspension became a clear solution that was stirred at room temperature. After 18 hours, when tic (pentane:ethyl acetate 1:2) indicated complete conversion into a higher migrating spot, the solution was neutralised with triethyl amine, concentrated and the residue was purified by silica gel flash column chromatography (toluene:ethyl acetate 3:2, 1:1) which gave 9.6 g, 56% of the title compound as white crystals.

NMR data 400 MHz (CDCl₃): ¹H, δ 1.38-1.43 (m, 2H), 1.56-1.75 (m, 8H), 2.43 (d, J=4.9 Hz, 1H), 3.28 (m, 1H), 3.75 (dd, J=3.9, 12.7 Hz, 1H), 3.82-3.94 (m, 3H), 4.05 (t, J=5.4 Hz, 1H), 4.22 (m, 1H). ¹³C, δ 23.9, 24.3, 25.2, 35.7, 38.3, 67.8, 68.7, 69.1, 71.9, 77.5, 110.5.

### 1,5-Anhydro-3,4-O-cyclohexylidene-L-ribulose (4)

A solution of dimethyl sulphoxide (2.65 ml, 37.3 mmol) in dichloromethane (30 ml) was added dropwise at −60° C. under nitrogen to a stirred solution of oxalyl chloride (1.79 ml, 20.5 mmol) in dichloromethane (30 ml) during a period of 15 min. To this solution a solution of 2,3-O-cyclohexylidene-1-deoxy-L-lyxopyroanose (4 g, 18.7 mmol) in dichloromethane (20 ml) was added dropwise during a period of 5 min. A white suspension was obtained and additional dichloromethane was added twice (10+30 ml). The temperature was allowed to rise to −25° C. where the suspension became a colourless solution. The temperature was again lowered to −45° C. and a solution of triethyl amine (12.9 ml, 93.3 mmol) in dichloromethane (20 ml) was added. After 10 min, when tic (toluene:ethyl acetate 1:1) indicated complete conversion of the alcohol into the ketone, the reaction mixture was poured into water (100 ml), the water layer was extracted once with dichloromethane (50 ml), the combined organic phases were dried with sodium sulphate, filtered and concentrated. Flash column chromatography on silica gel (eluent pentane:diethyl ether 1:1) of the residue gave a colourless solid. 3.4 g, 86%.

The oxidation was also performed by the Dess-Martin procedure:

A suspension of 2,3-O-cyclohexylidene-1-deoxy-L-lyxopyroanose (0.5 g, 2.33 mmol) and Dess-Martin periodinane (1.39 g, 3.29 mmol) in dichloromethane (5 ml) was stirred for 10 min then “wet dichloromethane” (46 □l water in 10 ml dichloromethane) was added dropwise during 15 min. After 1 h tic (toluene:ethyl acetate 1:1) indicated complete conversion of the starting material into a higher migrating spot. The reaction mixture was diluted with diethyl ether (100 ml) and washed with an aqueous solution of sodium hydrogen carbonate/sodium thiosulphate 1:1 (50 ml), dried with sodium sulphate, filtered and concentrated. Purification of the residue by flash column chromatography on silica gel (eluent pentane:diethyl ether 1:1) gave the title compound, 0.42 g, 84%, as a crystalline solid.

NMR data 400 MHz (CDCl₃): ¹H, δ 1.39-1.43 (m, 2H), 1.56-1.72 (m, 8H), 3.92-4.07 (m, 3H), 4.18-4.23 (m, 1H), 4.45 (d, J=6.8 Hz, 1H), 4.64-4.67 (m, 1H). ¹³Cδ 23.9, 24.1, 25.1, 35.3, 36.8, 68.5, 74.1, 75.1, 76.3, 112.4, 205.0.

### 1,5-Anhydro-4-deoxy-4-ethylidene-2,3-O-cyclohexylidene-D-erythro-pentitol (5)

Potassium-t-butoxide (3.41 g, 30.4 mmol) was added in one portion to a stirred suspension of ethyltriphenylphosphonium bromide (11.9 g, 32.0 mmol) in THF (60 ml) at −10° C. under nitrogen. The obtained orange-red mixture was allowed to reach room temperature, then cooled again to −10° C. and a solution of 1,5-anhydro-3,4-O-cyclohexylidene-L-ribulose (3.4 g, 16.0 mmol) in THF (40 ml) was added dropwise. The mixture was allowed to attain room temperature. 20 minutes after final addition, when tic (toluene:ethyl acetate 1:1) indicated complete conversion of the starting material into a higher migrating spot, the reaction mixture was partitioned between diethyl ether (400 ml) and water (200 ml). The organic layer was washed with water (1×200 ml) and brine (1×200 ml), dried with sodium sulphate, filtered and concentrated into a 10-ml residue. The residue was purified by flash column chromatography on silica gel (eluent pentane:ethyl acetate 95:5, 9:1) and appropriate fractions were carefully concentrated (bath temperature 25° C.) into a 10 g solution that was used directly in the next step.

### 1,5-Anhydro-4-deoxy-4-ethyl-2,3-O-cyclohexylidene-D-ribitol (6)

The above solution was diluted with ethyl acetate (30 ml), Pd/C ( 10%, 0.2 g) was added and the mixture was hydrogenated at atmospheric pressure. Additional Pd/C was added (0.16 g+0.20 g) after 40 and 90 minutes. After 100 minutes tic indicated almost complete consumption of the starting material. The reaction mixture was filtered through celite, concentrated into a liquid (5 ml) and purified by flash column chromatography on silica gel (eluent pentane:ethyl acetate 95:5, 9:1). Appropriate fractions were concentrated to 2.08 g and this solution was used directly in the next step.

NMR data 400 MHz (CDCl₃): ¹H, δ 0.98 (t, 3H), 1.31-1.74 (m, 12H), 1.82-1.92 (m, 1H), 3.18-3.26 (m, 2H), 3.64-3.68 (m, 1H), 3.84 (dd, J=6.4, 11.4 Hz, 1H), 4.08-4.14 (m, 1H), 4.27-4.29 (m, 1H). ¹³C, δ 11.3, 20.9, 24.0, 24.3, 25.3, 35.7, 38.3, 38.7, 67.7, 68.3, 70.8, 72.6, 109.5.

### 1,5-Anhydro-4-deoxy-4-ethyl-D-ribitol (7)

The above 1,5-anhydro-4-deoxy-4-ethyl-2,3-O-cyclohexylidene-D-ribitol was dissolved in aqueous acetic acid (80%, 25 ml) and the solution was stirred at 70° C. After 18 hours, when tic (pentane ethyl acetate 9:1 and 1:1) indicated almost complete consumption of the starting material (˜5% left), the solution was concentrated. Purification of the residue by flash column chromatography on silica gel (eluent pentane:ethyl acetate 1:1, 2:3) gave 0.91 g 39% (from the keto compound) of a colourless solid.

NMR data 400 MHz (CDCl₃): ¹H, δ 0.94 (t, 3H), 1.24-1.42 (m, 2H), 1.58-1.67 (m, 1H), 3.35 (t, 1H), 3.43 (t, 1H), 3.56 (dd, 1H), 3.67-3.71 (m, 2H). ¹³C, δ 11.4, 20.1, 42.1, 66.1, 66.3, 68.3, 68.7.

### 1,5-anhydro-2-O-benzyl-4-deoxy-4-ethyl-D-ribitol (8)

Sodium hydride (60%, 0.27 g, 6.84 mmol) was added in one portion, at room temperature, under nitrogen, to a stirred solution of 1,5-anhydro-4-deoxy-4-ethyl-D-ribitol (0.5 g, 3.42 mmol) in dimethylformamide (7 ml). After 30 minutes benzyl bromide (0.53 ml, 4.45 mmol) was added dropwise during 30 minutes. After 20 minutes, when tic (p.ether:ethyl acetate 4:1) indicated complete conversion of the diol, methanol (1 ml) was added and the mixture was stirred for 20 minutes. The reaction mixture was diluted with ethyl acetate (100 ml), washed with water (3×50 ml), dried with sodium sulphate, filtered and concentrated. Purification of residue by flash column chromatography on silica gel (eluent pentane:ethyl acetate 9:1, 4:1) gave 0.52 g, 64% of a colourless solid.

NMR data 400 MHz (CDCl₃): ¹H, δ 0.94 (t, 3H), 1.25-1.36 (m, 1H), 1.37-1.48 (m, 1H), 1.54-1.62 (m, 1H), 2.14 (s, 1H), 3.40 (t, 1H), 3.51-3.56 (m, 3H), 3.72-3.79 (m, 1H), 4.13 (s, 1H), 4.58 (d, J=11.7 Hz, 1H), 4.63 (d, J=11.7 Hz, 1H), 7.29-7.38 (m, 5H). ¹³C, δ 11.5, 20.1, 42.0, 64.1, 66.5, 66.6, 71.1, 75.6, 127.9, 128.2, 128.8, 138.1.

### 1,5-Anhydro-3-azido-2-O-benzyl-3,4-dideoxy-4-ethyl-D-xylitol (9)

Methanesulphonyl chloride (0.34 g, 2.96 mmol) was added to a stirred solution of 1,5-anhydro-2-O-benzyl-4-deoxy-4-ethyl-D-ribitol (0.28 g, 1.18 mmol) in pyridine (5 ml). The reaction mixture was warmed to 50° C. and stirred for one hour. Dichloromethane (100 ml) was added and the reaction mixture was washed successively with 1M aqueous sulphuric acid (2×50 ml), 1M aqueous sodium hydrogen carbonate, dried with sodium sulphate, filtered and concentrated. The residue was dissolved in dimethylformamide (10 ml) and sodium azide (0.31 g, 4.74 mmol) was added. The obtained mixture was stirred at 80° C. over night, diluted with ethyl acetate (100 ml), washed with water (3×50 ml), dried with sodium sulphate, filtered and concentrated. Purification of residue by flash column chromatography on silica gel (eluent toluene:ethyl acetate 95:5) gave 0.25 g, 81% of a colourless oil.

NMR data 400 MHz (CDCl₃): ¹H, δ 0.90 (t, 3H), 1.12-1.24 (m, 1H), 1.44-1.54 (m, 1H), 1.69-1.79 (m, 1H), 3.01 (t, 1H), 3.08-3.16 (m, 2H), 3.44-3.50 (m, 1H), 3.92 (dd, J=4.9, 11.7 Hz, 1H), 4.04 (ddd, J=1.0, 4.9, 11.2 Hz, 1H), 4.62 (d, J=11.7 Hz, 1H), 4.71 (d, J=11.2 Hz, 1H) 7.29-7.37 (m, 5H). ¹³C, δ 11.3, 22.0, 42.4, 68.5, 69.2, 70.9, 73.1, 78.2, 128.2, 128.2, 128.7, 138.0.

### 1,5-anhydro-3-[(tert-butoxycarbonyl)amino]-3,4-dideoxy-4-ethyl-D-xylitol (10)

Pd/C (10%, 30 mg) was added to solution of 1,5-anhydro-3-azido-2-O-benzyl-3,4-dideoxy-4-ethyl-D-xylitol (88 mg, 0.34 mmol) and di-tert-butyl dicarbonate (77 mg, 0.35 mmol) in ethyl acetate (4 ml) and the mixture was stirred under hydrogen. After 18 hours, when tic (pentane:ethyl acetate 9:1, ninhydride) indicated complete consumption of the starting material, the mixture was filtered through celite and concentrated. The residue was purified by flash column chromatography on silica gel (eluent toluene:ethyl acetate 4:1) which gave a colourless solid that still contained a benzyl group according to ¹H-nmr. The solid was dissolved in ethyl acetate:ethanol 1:1 and hydrogenated over Pd/C (10% 20 mg). After 1 hour, when tic (toluene ethyl acetate 1:1, ninhydride) indicated complete conversion of the starting material into a lower migrating spot, the mixture was filtered through celite and concentrated. Purification of residue by flash column chromatography on silica gel (eluent toluene:ethyl acetate 1:1, 2:3) gave 59 mg, 71% of the desired monool as a colourless solid. This monool is N-extended and capped as described herein and then oxidised to the corresponding pyranone. Alternatively the monool is first oxidised and then N-extended and capped.

NMR data (CDCl₃): ¹H, δ 0.90 (t, 3H), 1.12-1.24 (m, 1H), 1.42-1.52 (m, 10H), 1.59-1.70 (m, 1H), 3.05-3.16 (m, 2H), 3.26-3.30 (m, 1H), 3.43-3.48 (m, 2H), 3.96-4.05 (m, 2H). ¹³C, δ 11.5, 21.2, 28.5, 42.4, 59.2, 71.4, 71.8, 72.7.

Alternative method for the preparation of 5-methyl pyranones as building blocks and intermediates towards 5-functionalised pyranones

### 2-Benzyloxycarbonylamino-4-hydroxy-3-methyl-butyric acid tert-butyl ester

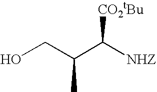

2-Benzyloxycarbonylamino-4-hydroxy-3-methyl-butyric acid tert-butyl ester was prepared following procedures reported by J. E. Baldwin et al (Tetrahedron 1995, 51(42), 11581).

### (4-Methyl-2-oxo-tetrahydro-furan-3-yl)-carbamic acid benzyl ester

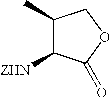

2-Benzyloxycarbonylamino-4-hydroxy-3-methyl-butyric acid tert-butyl ester (1.00 g, 3 mmol) was dissolved in TFA (30 mL). This solution was stirred for 45 minutes and then concentrated in vacuo. The residual TFA was removed azeotropically with toluene. This residue was purified by flash column chromatography to yield the title compound as a crystalline solid (750 mg, 80%), MS (ES⁺) 250 (M+H).

### [3-Hydroxy-1-(methoxy-methyl-carbamoyl)-2-methyl-propyl]-carbamic acid benzyl ester 4

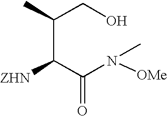

The lactone ring of (4-methyl-2-oxo-tetrahydro-furan-3-yl)-carbamic acid benzyl ester can be opened using N,O-dimethylhydroxylamine hydrochloride in the presence of Me₃Al to give the title compound.

### [3-tert-Butoxy-1-(methoxy-methyl-carbamoyl)-2-methyl-propyl]-carbamic acid benzyl ester 5

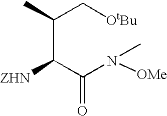

The primary alcohol of [3-hydroxy-1-(methoxy-methyl-carbamoyl)-2-methyl-propyl]-carbamic acid benzyl ester can be protected using tert-butyl-2,2,2-trichloroacetimidate and boron trifluoride etherate to give the title compound.

### (3-tert-Butoxy-1-formyl-2-methyl-propyl)-carbamic acid benzyl ester 6

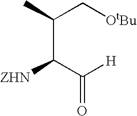

The Weinreb amide function of [3-tert-butoxy-1-(methoxy-methyl-carbamoyl)-2-methyl-propyl]-carbamic acid benzyl ester can be reduced using lithium aluminium hydride in ether to provide the title compound.

### 2-Benzyloxycarbonylamino-4-tert-butoxy-3-methyl-butyric acid 7

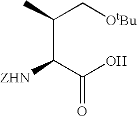

(3-tert-butoxy-1-formyl-2-methyl-propyl)-carbamic acid benzyl ester in tert-butyl alcohol in the presence of 2-methyl-2-butene can be oxidised using a solution of sodium chlorite and monobasic sodium phosphate in water to give the title compound.

### [3-tert-Butoxy-1-(2-diazo-acetyl)-2-methyl-propyl]-carbamic acid benzyl ester 9

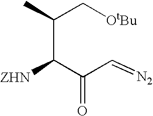

Activation of 2-benzyloxycarbonylamino-4-tert-butoxy-3-methyl-butyric acid with isobutyl chloroformate and 4-methylmorpholine, and subsequent treatment of the activated acid with diazomethane allows for the preparation of the title compound.

### (3-Methyl-5-oxo-tetrahydro-pyran-4-yl)-carbamic acid benzyl ester 10

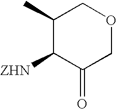

Cyclisation of tert-butoxy-1-(2-diazo-acetyl)-2-methyl-propyl]-carbamic acid benzyl ester using lithium chloride in aqueous acetic acid gives the title compound. The CBz protecting group is readily replaced with Boc or Fmoc etc by conventional protecting group manipulation.

### Alternative Route Towards Chiral β-Alkyl Serines

Following the chemistry detailed in scheme 3. Exemplified by the synthesis of (2S,3S)-β-hydroxynorvaline (15) (also termed of (2S,3S)-β-ethylserine)

### (a) Tri-acetone-D-mannitol

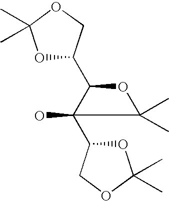

D-Mannitol (49.5 g, 0.27 mol) was suspended in acetone (600 mL, 99.9% purity). To the suspension H₂SO₄ (4.95 mL) was added and the mixture shaken at 21° C. overnight. The solution was then filtered and the clear solution neutralised with a saturated solution of NaHCO₃ until pH=6. The solvent was concentrated in vacuo, affording tri-acetone-D-mannitol as a white solid, yield 78 g, 96%. Electrospray-MS m/z 303 (MH⁺).

### (b) 3,4-Isopropylidine-D-mannitol

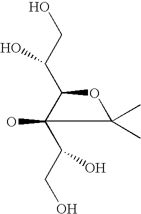

Tri-acetone-D-mannitol (78 g, 0.26 mol) was dissolved in the minimum amount of 70% acetic acid (400 mL) and stirred in water bath at 42.7° C. for 1.5 hrs. The solvent was quickly evaporated in vacuo to give 3,4-Isopropylidine-D-mannitol as a colourless oil, yield 57.6 g, 99.8%. Electrospray-MS m/z 223 (MH⁺).

### (c) 1,2,5,6-tetra-O-benzyl-3,4-O-isopropylidine-D-mannitol (35)

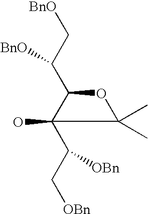

3,4-Isopropylidine-D-mannitol (57.64 g, 0.26 mol) was dissolved in benzylchloride (543 mL). To the stirred solution powdered KOH (500 g) was added and the solution heated in an oil bath at 133° C. for 2 hrs. The mixture was allowed to cool to room temperature and poured into a 300 mL beaker. Ice and water (1400 mL) were carefully added, the mixture extracted with DCM (800 mL) and the aqueous phase further extracted with DCM (300 mL). The organic extracts were dried over sodium sulphate and the filtered solution concentrated in vacuo. The residue was purified by flash chromatography over silica gel eluting with EtOAc/heptane (1:15 to 1:10, v/v) to afford compound (35) as a colourless oil, yield 77 g, 51%.

Electrospray-MS m/z 583 (MH⁺). Analytical HPLC Rt=29.16 mins (91.8%). □_{H} (500 MHz, CDCl₃) 1.35 (6H, s, C(CH₃)₂), 3.62 (2H, dd, J 6, 10, 2×CHOC), 3.75 (4H, m, 2×CH₂OBn), 4.15-4.20 (1H, m, CHOBn), 4.46 (1H, dd, J 12.5, 14.5, CHOBn), 4.77 (4H, d, J 11.5, 4×CH_{2A}C₆H₅), 4.73 (4H, d, J 11.5, 4×CH_{2B}C₆H₅) and 7.25-7.34 (20H, m, 4×C₆H₅).

### (d) 1,2,5,6-Tetra-O-benzyl-D-mannitol (36)

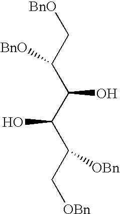

In a 2000 mL flask fitted with a condenser compound (35) (41.11 g, 0.071 mol) was dissolved in 70% acetic acid (700 mL) and the solution stirred at 100° C. in an oil bath for 1.5 hrs. After concentration in vacuo, the residue was purified by flash chromatography over silica gel eluting with EtOAc/heptane (3:7, v/v) to afford compound (36) as a pale yellow oil, yield 21.8 g, 57%.

Electrospray-MS m/z 543 (MH⁺). Analytical HPLC Rt=25.8 (100%). δ_{H} (500 MHz, CDCl₃) 3.01 (2H, d, J 6.0, 2×OH), 3.65-3.70 (2H, m, 2×CHOBn), 3.72-3.78 (4H, m, 2×CH₂OBn), 3.93-3.97 (2H, m, 2×CHOH), 4.55 (4H, s, 2×CH₂C₆H₅), 4.73 (2H, d, J 11.5, 2×CH_{2A}C₆H₅), 4.77 (2H, d, J 11.5, 2×CH_{2B}C₆H₅) and 7.25-7.34 (20H, m, 4×C₆H₅).

### (e) (2R)-2,3-Di-O-benzylglyceraldehyde (37)

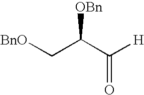

Compound (36) (10.78 g, 0.02 mol) was dissolved in anhydrous toluene (150 mL). While vigorously stirring lead tetraacetate (9.83 g, 0.023 mol, 1.1 eq) was added as a solid and the mixture stirred for 3 hrs at room temperature. The mixture was then filtered and the filtered concentrated in vacuo to afford compound (37) as a colourless oil, yield 10.2 g, 95%.

δ_{H} (500 MHz, CDCl₃) 3.75-3.83 (2H, m, CH₂OBn), 3.97 (1H, t, J 4, CHOBn), 4.55 (2H, d, J 5.5, 2×CH_{2A}C₆H₅), 4.70 (2H, d, J 12, 2×CH_{2B}C₆H₅), 7.20-7.40 (10H, m, 2×C₆H₅) and 9.70 (1H, s, CHO).

### (f) (2S)—N-(2,3-Dibenzyloxypropylidene)benzylamine (38)

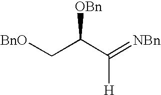

Benzylamine (4.06 mL, 0.037 mol, 1 eq) was dissolved in anhydrous diethyl ether (150 mL) and the solution cooled to 0° C. To a solution of compound (37) (9.9 g, 0.037 mol, 1 eq) in anhydrous diethyl ether (100 mL) at 0° C., was added anhydrous magnesium sulphate (7.3 g) and the solution transferred via cannula under argon to the solution of the amine. After stirring for 3 hrs the reaction mixture was concentrated in vacuo to give compound (38) as a crude colourless oil, yield 12.2 g, 96%.

δ_{H} (500 MHz, CDCl₃) 3.75-3.83 (2H, m, CH₂OBn), 4.17-4.25 (1H, m, CHOBn), 4.57 (2H, s, NCH₂C₆H₅), 4.62 (2H, m, 2×CH_{2A}C₆H₅), 4.70 (2H, m, CH_{2B}C₆H₅), 7.20-7.40 (15H, m, 3×C₆H₅) and 7.70 (1H, m, CHN).

### (g) (1R,2S)—N-Benzyl-2,3-dibenzyloxy-1-phenyl-1-propylamine (39)

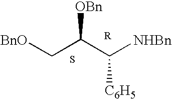

Phenylmagnesium bromide (29.17 mL, 0.087 mol, 3.0M, 2.5 eq) was dissolved in anhydrous diethyl ether (124 mL) and the solution cooled to 0° C. under argon. A solution of compound (38) (12.5 g, 0.035 mol) in anhydrous diethyl ether (140 mL) was transferred via cannula to the solution of the phenylmagnesium bromide and the reaction mixture stirred at room temperature for 2 hrs. The solution was poured into an aqueous solution of NH₄Cl (200 mL) and extracted with tert-butyl methyl ether (2×100 mL). The combined extracts, dried over anhydrous sodium sulphate, were concentrated in vacuo. The crude oil obtained was purified by flash chromatography over silica gel eluting with EtOAc/heptane (1:4, v/v) to afford compound (39) as a pale yellow oil, yield 8.5 g, 56%. Electrospray-MS m/z 438 (MH⁺). Analytical HPLC Rt=24.0 mins (98%).

δ_{H} (500 MHz, CDCl₃) 2.45 (1H, br s, NH), 3.32 (1H, dd, J 10, 4.5, CH_{2A}OBn), 3.43 (1H, d, J 13, C₆H₅CH_{2A}NH) 3.50-3.54 (1H, dd, J 10, 3, CH_{2B}OBn), 3.55-3.61 (1H, d, J 13, C₆H₅CH_{2B}NH), 3.65-3.78 (1H, m, CHOBn), 3.90 (1H, d, J 7, C₆H₅CHNH), 4.40 (2H, s, OCH₂C₆H₅), 4.62 (1H, d, J 11, OCH_{2A}C₆H₅), 4.70 (1H, d, J 11, OCH_{2B}C₆H₅) and 7.18-7.40 (20H, m, 4×C₆H₅).

### (h) (1R,2S)—N-Benzyl-tert-butoxycarbonyl-2,3-dibenzyloxy-1-phenyl-1-propylamine (40)

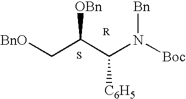

Compound (39) (9.26 g, 0.02 mol) was dissolved in dioxane (66 mL) and diisopropylamine (0.37 mL, 0.0021 mol, 0.11 eq) was added. To the stirred solution di-tert-butyl dicarbonate (11.25 g, 0.0516 mol, 2.6 eq) was added as a solid and the solution stirred at 50° C. in an oil bath overnight. The mixture was treated with tert-butyl methyl ether (300 mL), washed with 1.0M KHSO₄ aqueous solution (60 mL) and the organic extracts were dried over anhydrous sodium sulphate and concentrated in vacuo. The crude oil was purified by flash chromatography over silica gel eluting with EtOAc/heptane (1:9, v/v) to afford compound (40) as a colourless oil, yield 7.7 g, 71%. Electrospray-MS m/z 538 (MH⁺). Analytical HPLC Rt=30.0 mins (95%).

δ_{H} (500 MHz, CDCl₃) 1.30 (9H, s, C(CH₃)₃), 3.44 (1H, dd, J 10, 4.5, CH_{2A}OBn), 3.61 (1H, dd, J 10, 2, CH_{2B}OBn), 4.30 (1H, m, CH_{2A}N) 4.37 (2H, d, J 12, OCH_{2A}C₆H₅), 4.43 (2H, d, J 12, OCH_{2B}C₆H₅), 4.50-4.63 (1H, m, CH_{2B}N), 4.85 (1H, m, CHOBn) 5.25 (1H, d, J 9, C₆H₅CHN) and 7.00-7.45 (20H, m, 4×C₆H₅).

### (i) (1R,2S)—N-tert-Butoxycarbonyl-2,3-hydroxy-1-phenyl-1-propylamine (41)

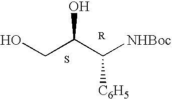

Compound (40) (7.67 g, 0.014 mol) was dissolved in anhydrous methanol (80 mL). After having flushed the flask with argon, 20% Pd(OH)₂/C (10.00 g, Degussa type, E101 NE/W, wet) was carefully added and the mixture stirred under H₂ for 48 hrs. The mixture was carefully filtered through a pad of Celite and the catalyst washed with a solution of aqueous methanol (10:100 H₂O:CH₃OH, v/v). The filtered solution was concentrated in vacuo and the residue purified by flash chromatography over silica gel eluting with EtOAc/heptane (3:1, v/v) to afford compound (41) as a colourless oil, yield 2.7 g, 72%. Electrospray-MS m/z 268 (MH⁺). Analytical HPLC Rt=15.3 mins (100%).

δ_{H} (500 MHz, CDCl₃) 1.44 (9H, s, C(CH₃)₃), 2.6 (2H, br s, OH), 3.56 (2H, d, J 5.5, CH₂OH), 3.97 (1H, s, C₆H₅CHNH), 4.83 (1H, s, C₆H₅CHCHOH), 5.28 (1H, d, J 8, NH) and 7.20-7.45 (5H, m, C₆H₅).

### (j) (1R,2S)—N-tert-Butoxycarbonyl-3-tert-butyldimethylsilyloxy-2-hydroxy-1-phenyl-1-propylamine (42)

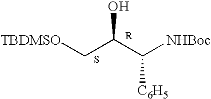

Compound (41) (2.67 g, 0.01 mol) was dissolved in anhydrous DMF (60 mL) and stirred under argon. Imidazole (1.5 g, 0.022 mol, 2.2 eq) was added followed by the addition of TBDMSCI (1.66 g, 0.011 mol, 1.1 eq). The reaction mixture was stirred overnight at room temperature. The mixture was diluted with ether (240 mL), washed with saturated NH₄Cl (120 mL) and H₂O (40 mL) and the aqueous layer extracted with ether (4×100 mL). The combined extracts were dried over anhydrous sodium sulphate, filtered and concentrated in vacuo. Purification of the residue by flash chromatography over silica gel eluting with EtOAc/heptane (3:1, v/v) afforded compound (42) as a colourless oil, yield 3.31 g, 87%. Electrospray-MS m/z 382 (MH⁺).

δ_{H} (500 MHz, CDCl₃) 0.05 (3H, s, CH_{3A}SiCH₃), 0.06 (3H, s, CH₃SiCH_{3B}), 0.89 (9H, s, Si(CH₃)₃), 1.39 (9H, br s, C(CH₃)₃), 2.45 (1H, br s, OH), 3.51 (1H, dd, J 10, 7, TBDMSOCH_{2A}), 3.65 (1H, dd, J 10, 4.5, TBDMSOCH_{2B}), 3.85 (1H, m, CHOH), 4.66 (1H, m, C₆H₅CHNH), 5.45 (1H, br s, NH) and 7.23-7.35 (5H, m, C₆H₅).

### (k) (1R,2S)—N-tert-butoxycarbonyl-3-tert-butyldimethylsilyloxy-2-mesyloxy-1-phenyl-1-propylamine (43)

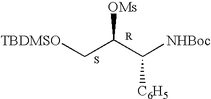

Compound (42) (1.30 g, 3.40 mmol, 1.0 eq) was dissolved in anhydrous DCM (30 mL). To the solution TEA (0.57 mL, 4.09 mmol, 1.2 eq) was added and the mixture was cooled to 0° C. in an ice-water bath. At this temperature and under argon, a solution of MsCl (0.32 ml, 4.09 mmol, 1.2 eq) in anhydrous DCM (3 mL) was added. The mixture was stirred for 1.5 hrs. The reaction mixture was treated with water (20 mL) and extracted with DCM (20 mL). The aqueous phase was further extracted with DCM (4×60 mL) and the combined organic layers were dried over anhydrous sodium sulphate and concentrated in vacuo. The residue was purified by flash chromatography over silica gel eluting with EtOAc/heptane (1:3, v/v) affording compound (43) as a colourless oil, yield 1.30 g, 83%. Electrospray-MS m/z 460 (MH⁺). Analytical HPLC Rt: 27.1 mins (98%).

δ_{H} (500 MHz, CDCl₃) 0.06 (3H, s, CH_{3A}SiCH₃), 0.07 (3H, s, CH₃SiCH_{3B}), 0.91 (9H, s, Si(CH₃)₃), 1.42 (9H, br s, C(CH₃)₃), 2.54 (3H, s, SO₂CH₃), 3.77 (2H, d, J 6.0, TBDMSOCH₂), 4.7 (1H, m, CHOH), 5.1 (1H, m, C₆H₅CHNH), 5.4 (1H, br s, NH) and 7.26-7.38 (5H, m, C₆H₅).

### (l) (1R,2R)—N-tert-Butoxycarbonyl-2,3-epoxy-1-propylamine (44)

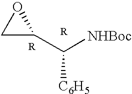

Compound (43) (3.79 g, 8.26 mmol, 1.0 eq) was dissolved in THF anhydrous (78 mL) and the solution cooled to 0° C. in an ice water bath. TBAF (16.52 mL, 1.0M sol in THF, 16.52 mmol, 2 eq) was added dropwise via syringe and once the addition was complete the ice bath was removed. The reaction mixture was stirred at room temperature overnight and then treated with water (40 mL), extracted with diethyl ether (40 mL) and the aqueous phase further extracted with diethyl ether (3×75 mL). The combined extracts were dried over anhydrous sodium sulphate, filtered and concentrated in vacuo. The residue was purified by flash chromatography over silica gel eluting with TBME/heptane (1:6 to 2:1, v/v) affording compound (44) a white solid, yield 1.0 g, 48%.Electrospray-MS m/z 250 (MH⁺).

δ_{H} (500 MHz, CDCl₃) 1.42 (9H, s, C(CH₃)₃), 2.50 (1H, dd, J 5, 2.2, CHCH_{2A}O), 2.76 (1H, dd, J 5, 4, CHCH_{2B}O), 3.20-3.30 (1H, m, CHCH₂O), 4.72 (1H, br s, C₆H₅CHCHO), 5.00(1H, br s, NH) and 7.27-7.38(5H, m, C₆H₅).

### (m) (1R,2S)—N-tert-butoxycarbonyl-2-hydroxy-1-phenyl-1-butylamine (45)

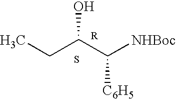

Copper(I)iodide (0.574 g, 3.01 mmol, 5 eq) was dispersed in anhydrous diethyl ether (17 mL). After cooling the suspension to −35° C. under argon, CH₃Li in diethyl ether (3.76 mL, 1.6M, 6.02 mmol, 10 eq) was added dropwise. After stirring at −35° C. for 30 mins a solution of compound (44) (0.15 g, 0.60 mmol, 1.0 eq) dissolved in diethyl ether (1.5 mL) was added dropwise to the solution of the organocuprate and the reaction mixture was stirred at −35° C. for 1.5 hrs. Ethyl acetate (12.5 mL) was added followed by the careful addition of a saturated solution of NH₄Cl (10 mL) and water (3 mL). The mixture was allowed to warm up to room temperature and the organic phase extracted. The aqueous phase was further extracted with ethyl acetate (3×15 mL) and the combined extracts dried over anhydrous sodium sulphate, filtered and concentrated in vacuo. The crude oil was purified by flash chromatography over silica gel eluting with TBME/heptane (2:3, v/v) affording compound (45) as a white solid, yield 0.14 g, 88%. Electrospray-MS m/z 266 (MH⁺). Analytical HPLC Rt=17.6 mins (100%).

δ_{H} (500 MHz, CDCl₃) 0.97 (3H, t, J 7.5, CH₃CH₂), 1.10-1.25 (1H, m, CH₃CH_{2A}), 1.25-1.50 (1H, m, CH₃CH_{2B}), 1.50 (9H, s, C(CH₃)₃), 3.78 (1H, br s, CHOH), 4.73 (1H, br s, C₆H₅CHNH), 5.28 (1H, br s, NH) and 7.25-7.38 (5H, m, C₆H₅).

### (n) (1R,2S)—N-tert-Butoxycarbonyl-2-tert-butoxy-1-phenyl-1-butylamine (46)

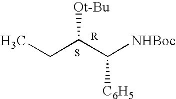

In a sealed tube, compound (45) (0.114 g, 0.43 mmol) was dissolved in anhydrous DCM (11 mL). Whilst stirring was maintained, the tube was immersed in a dry ice-acetone bath and cooled to −60° C. Isobutylene (11 mL) was condensed into the tube and methyltriflate (55 □L) was carefully added. The tube was capped tightly and the bath removed to allow the reaction to proceed at room temperature for 4 days. The tube was cooled to −60° C., the lid removed and then the bath removed to allow the excess of isobutylene to slowly evaporate whilst warming up to room temperature. At about 10° C., TEA (0.7 mL) was added to neutralise the excess acid. The residue obtained after removal of the solvents in vacuo was purified by flash chromatography over silica gel eluting with EtOAc/heptane (2:8, v/v) affording compound (46) as a white solid, yield 0.02 g, 14% Electrospray-MS m/z 322 (MH⁺). Analytical HPLC Rt=24.1 mins (90%).

δ_{H} (500 MHz, CDCl₃) 0.84 (3H, t, J 7.5, CH₃CH₂), 1.15-1.30 (1H, m, CH₃CH_{2A}) 1.24 (9H, s, CHOC(CH₃)₃), 1.35-1.40 (1H, m, CH₃CH_{2B}), 1.41 (9H, s, CO₂C(CH₃)₃), 3.72 (1H, m, CHO(CH₃)₃), 4.78 (1H, m, C₆H₅CHNH), 5.15 (1H, br s, NH) and 7.22-7.38 (5H, m, C₆H₅).

### (o) (2S,3S)—N-tert-Butoxycarbonyl-□-tert-butoxy-norvaline (47)

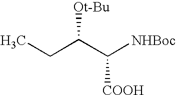

Compound (46) (0.024 g, 0.074 mmol, 1 eq), was dissolved in a mixture of CCl₄/CH₃CN/H₂O (1:1:2, v/v/v, 2.4 mL). To the stirred biphasic solution NaHCO₃ (0.104 g, 1.25 mmol, 16.9 eq) was added as a solid, followed by the careful addition of NaIO₄ (0.284 g, 1.33 mmol, 18 eq). After 10 minutes RuCl₃.3H₂O (1.5 mg, 7.23 □mol, 0.1 eq) was added and the reaction mixture stirred for 48 hrs. The solution was treated with EtOAc (15 mL) and acidified to pH=3 by dropwise addition of citric acid (10%). The organic phase was further extracted with EtOAc (3×15 mL) and the combined extracts were dried over anhydrous magnesium sulphate, filtered and concentrated in vacuo. The crude residue was purified by flash chromatography over silica gel eluting with a gradient of MeOH/CH₃Cl (0.1:10 to 1.0:10, v/v) to give compound (47) as a white solid, yield 0.009 g, 42%.

Electrospray-MS m/z 290 (MH⁺).

### (p) (2S,3S)-β-Hydroxy-norvaline (15)

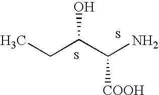

Compound (47) (9 mg, 0.03 mmol) was dissolved in a solution HCl in dioxane (1 mL, 4.0M). After stirring for 3 hrs at room temperature, the solvent was removed in vacuo and the residue was lyophilised using CH₃CN/H₂O (4:1, v/v) to yield (2S,3S)-□-hydroxynorvaline (15) as a white solid, 3.0 mg, 75%.Electrospray-MS m/z 134 (MH⁺).

□_{H} (500 MHz; CD₃OD) 1.00 (3H, t, J 7.5, CH₃CH₂), 1.50-1.65 (2H, m, CH₃CH₂), 3.88-3.95 (1H, m, CHOH) and 3.98 (1H, d, J 3, C₆H₅CHNH₂).

### Chemistry Towards P2 Hybrid Aminoacids

The general chemistry depicted in scheme 4 will shortly be published in full in the academic literature, by its inventors CS Dexter and RFW Jackson at the University of Newcastle, England.

### (a) General Procedure for the Zinc Coupling Reactions

### (b) Zinc Activation

Zinc dust (150 mg, 2.3 mmol, 3.0 eq, Aldrich) was weighed into a 25 mL round bottom flask with a side arm and fitted with a three way tap. The zinc powder was heated with a heat gun under vacuum and the flask was flushed with nitrogen and evacuated and flushed a further three times. With the flask filled with nitrogen, dry DMF (1 mL) was added. Trimethylsilylchloride (30 μl, 0.23 mmol, 0.3 eq) was added and the zinc slurry was vigorously stirred for a further 30 mins.

### (c) Zinc Insertion; N-(tert-Butoxycarbonyl)-3-iodozinc-L-alanine methyl ester (61)

N-(tert-Butoxycarbonyl)-3-iodo-L-alanine methyl ester (247 mg, 0.75 mmol, 1.0 eq) dissolved in dry DMF (0.5 mL) was added dropwise, via cannula, to the activated zinc slurry at 0° C. prepared as described above. The reaction mixture was then allowed to warm up to room temperature and stirred for 1 hr to give the organozinc reagent.

### (d) CuBr.SMe₂ Preparation

Whilst the zinc insertion reaction was in progress, CuBr.SMe₂ (20 mg, 0.1 mmol, 0.13 eq) was weighed into a 25 ml round bottom flask fitted with a three way tap and dried “gently” with a heat gun under vacuum until CuBr.SMe₂ changed appearance from a brown powder to give a light green powder. Dry DMF (0.5 mL) was then added followed by addition of the electrophile (either 1-bromo-2-methylbut-2-ene, toluene-4-sulfonic acid-(E)-2-methyl-but-2-enyl ester or 1-bromo-2,3-dimethylbut-2-ene) (1.0 mmol, 1.3 eq). The reaction mixture was then cooled to −15° C.

### (e) Coupling Reaction

Stirring of the organozinc reagent solution was stopped to allow the zinc powder to settle and the supernatant was carefully removed via cannula (care taken to avoid transferring too much zinc powder) and added dropwise to the solution of electrophile and copper catalyst. The cooling bath was removed and the solution was stirred at room temperature overnight. Ethyl acetate (20 mL) was added and stirring was continued for a further 15 mins. The reaction mixture was transferred to a separating funnel and a further aliquot of EtOAc (30 mL) was added. The organic phase was washed successively with 1M Na₂S₂O₃ (20 mL), water (2×20 mL), brine (40 mL), dried over sodium sulphate and filtered. The solvent was removed in vacuo and the crude product purified by flash chromatography on silica gel as described.

### (f) Hydrogenation of Alkene

The alkene (1.0 mmol) was dissolved in ethanol (10 mL), 10% palladium on carbon (80 mg) added and hydrogen introduced. Once the reaction had been deemed to have reached completion, the hydrogen was removed, the reaction filtered through Celite and the catalyst washed with ethanol (30 mL). The combined organic filtrate was concentrated in vacuo and the alkane used directly in the subsequent reaction.

### (g) Saponification of Methyl Ester

The methyl ester (1.0 mmol) was dissolved in THF (6 mL) and whilst stirring, a solution of LiOH (1.2 mmol, 1.2 eq) in water (6 mL) was added dropwise. Once the reaction was deemed to have reached completion, the THF was removed in vacuo and diethyl ether (10 mL) added to the residue. The reaction mixture was then acidified with 1.0M HCl until pH=3. The organic phase was then removed and the aqueous layer extracted with diethyl ether (2×10 mL). The combined organic extracts were dried over magnesium sulphate, filtered and the solvent removed in vacuo to give the carboxylic acid used directly in the subsequent reaction.

### (h) Removal of N-Boc Protecting Group

The N-Boc protected material (1.0 mmol) was dissolved in DCM (2 mL) and cooled to 0° C. Trifluoroacetic acid (2 mL) was added dropwise and when the reaction was deemed to have reached completion, the solvents were removed in vacuo to yield the amine used directly in the subsequent reaction. Alternatively, the N-Boc protected material (1.0 mmol) was cooled to 0° C. and 4M HCl in dioxane (5 mL) added dropwise and when the reaction was deemed to have reached completion, the solvents were removed in vacuo to yield the amine used directly in the subsequent reaction.

### (i) Fmoc Protection of Amine

The amine (1.0 mmol) in 1,4-dioxane (2 mL) was cooled to 0° C. and 10% sodium carbonate (2.2 mmol, 2.2 eq, 2 mL) added. The biphasic reaction mixture was stirred vigorously and Fmoc-Cl (1.1 mmol, 1.1 eq) added. Once the reaction was deemed to have reached completion, diethyl ether (10 mL) added and the reaction mixture acidified to pH=3 with 1M HCl. The organic phase was removed and the aqueous layer extracted with diethyl ether (2×10 mL). The combined organic extracts were dried over sodium sulphate, filtered, the solvent removed in vacuo and the residue purified by flash chromatography over silica gel.

### EXAMPLE SYNTHESIS 1

### Preparation of 2S-2-(9H-fluoren-9-ylmethoxycarbonylamino)-4,4-dimethylhexanoic acid (68)

The following scheme explains how optically pure (S)-2-tert-Butoxycarbonylamino-4,4-dimethyl-hex-5-enoic acid methyl ester (62) was prepared and isolated.

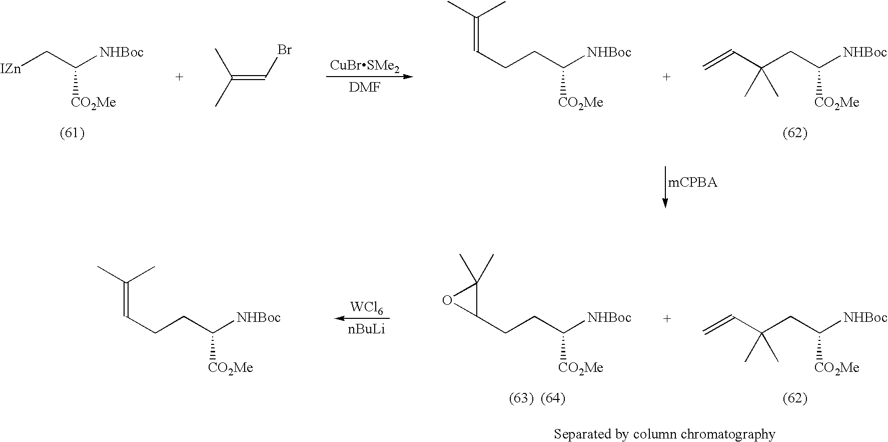

(a) 2S-2-tert-Butoxycarbonylamino-4,4-dimethyl-hex-5-enoic acid methyl ester (62), 2S-2-tert-butoxycarbonylamino-4-(2S-3,3-dimethyl-oxiranyl)-butyric acid methyl ester (63) and 2S-2-tert-butoxycarbonylamino-4-(2R-3,3-dimethyl-oxiranyl)-butyric acid methyl ester (64)

Following the general procedure for zinc coupling reactions, 1-bromo-3-methylbut-2-ene (115 μL, 1.0 mmol) was coupled to compound (61) (247 mg, 0.75 mmol) in the presence of CuBr.SMe₂ (20 mg, 0.1 mmol) to give a residue which was purified by flash column chromatography over silica gel eluting with EtOAc/40:60 petroleum ether (1:9, v/v). Fractions were pooled and reduced in vacuo to give a mixture of regioisomers (2:1 formal SN2′ vs SN2), inseparable by column chromatography, as a colourless oil, yield 190 mg, 93%.

To a mixture of regioisomers (190 mg, 0.7 mmol) in chloroform (3 mL) was added dropwise over 5 mins, 3-chloroperbenzoic acid (156 mg, 85% pure, 0.8 mmol, 1.1 eq) in chloroform (2 mL). The reaction mixture was stirred at room temperature for a further 2 hr. The reaction mixture was then washed successively with 1M Na₂S₂O₅ (5 mL), saturated sodium bicarbonate solution (5 mL) and brine (10 mL). The organic phase was dried over sodium sulfate, filtered, the solvent removed in vacuo and the residue was purified by flash chromatography over silica gel eluting with EtOAc/40:60 petroleum ether (2:8, v/v). Three products were obtained; compound (62) was eluted first and further elution afforded an inseparable mixture of compound (63) and compound (64). Fractions of the initial component were pooled and reduced in vacuo to give 2S-2-tert-butoxycarbonylamino-4,4-dimethyl-hex-5-enoic acid methyl ester (62) as a clear oil, yield 93 mg, 49%. Electrospray-MS m/z 272 (MH⁺). Analytical HPLC Rt=21.45 mins (95%), HRMS C₁₀H₁₇O₄N requires M, 215.1158, found: M⁺-C₄H₈ 215.1152 (□-2.8 ppm); IR (cap. film)/cm⁻¹ 3369 (s), 3084 (m), 2965 (s), 1748 (s), 1715 (s), 1517 (s), 1167 (s), 1007 (s), 914 (s)

δ_{H} (500 MHz; CDCl₃) 1.06 (6H, s, CH₂═CHC(CH₃)₂), 1.42 (9H, s, C(CH₃)₃) 1.55 (1H, dd, J 14, 9, CH₂═CHC(CH₃)₂CH_{2A}), 1.82 (1H, dd, J 14, 3, CH₂═CHC(CH₃)₂CH_{2B}), 3.69 (3H, s, OCH₃), 4.30 (1H, m, NHCHCO₂CH₃), 4.83 (1H, br d, J 7, NH), 4.97 (2H, m, CH₂═CH) and 5.78 (1H, dd, Jₜᵣₐₙₛ 17.5, J_{cis} 11, CH₂═CH) δ_{C}(125 MHz; CDCl₃) 26.93 (CH₂═CHC(CH₃)₂), 28.34 (C(CH₃)₃), 36.33 (CH₂═CHC(CH₃)₂CH₂), 45.06 (CH₂═CHC(CH₃)₂), 51.25 (NHCHCO₂CH₃), 52.15 (OCH₃), 79.77 (C(CH₃)₃), 111.39 (CH₂═CH), 146.87 (CH₂═CH), 154.97 (NHCO₂Bu^{t}) and 174.04 (CO₂CH₃).

### (b) 2S-2-tert-Butoxycarbonylamino-4,4-dimethyl-hexanoic acid methyl ester (65)

Following the general procedure for alkene hydrogenation, compound (62) (93 mg, 0.3 mmol) yielded compound (65) as a colourless oil, yield 90 mg, 96% and used directly in the subsequent reaction. Electrospray-MS m/z 274 (MH⁺). Analytical HPLC Rt=22.55 mins (100%).

### (c) 2S-2-tert-Butoxycarbonylamino-4,4-di methyl-hexanoic acid (66)

Following the general procedure for methyl ester saponification, compound (65) (90 mg, 0.3 mmol) gave compound (66) as crystals, yield 79 mg, 92% and used directly in the subsequent reaction. Electrospray-MS m/z 260 (MH⁺). Analytical HPLC Rt=20.90 mins (100%).

### (d) 2S-2-Amino-4,4-dimethyl-hexanoic acid trifluoroacetic acid salt (67)

Following the general procedure of N-Boc removal using TFA, compound (66) (79 mg, 0.3 mmol) gave compound (67) as a solid, yield 80 mg, 96% and used directly in the subsequent reaction. Electrospray-MS m/z 274 (MH⁺).

### (e) 2S-2-(9H-Fluoren-9-ylmethoxycarbonylamino)-4,4-dimethyl-hexanoic acid (68)

Following the general procedure for Fmoc protection of an amine, compound (67) (80 mg, 0.3 mmol) gave on purification by flash chromatography over silica gel eluting with CHCl₃/CH₃OH (100:0 to 96:4, v/v) 2S-2-(9H-fluoren-9-ylmethoxycarbonylamino)-4,4-dimethyl-hexanoic acid (68) as a solid, yield 60 mg, 54%. Electrospray-MS m/z 382(MH⁺). Analytical HPLC Rt=23.63 mins (100%); [α]_{D}¹⁷−18.4 (c 0.25 in EtOH)

δ_{H} (500 MHz, CDCl₃) 0.88 (3H, t, J 7, CH₃CH₂), 0.95 (6H, s, CH₃CH₂C(CH₃)₂), 1.31 (2H, m, CH₃CH₂), 1.46 (1H, dd, J 14.5, 10, CH₃CH₂C(CH₃)₂CH_{2A}), 1.85 (1H, br d, J 14.5, CH₃CH₂C(CH₃)₂CH_{2B}), 4.21_(1H, t, J 6.5, CH-Fmoc), 4.41 (3H, m, NHCHCO₂H and CH₂O), 5.02 (1H, br d, J 8, NH-Fmoc), 7.29 (2H, m, H-2′ and H-7′), 7.38 (2H, m, H-3′ and H-6′), 7.58 (2H, m, H-1′ and H-8′) and 7.74 (2H, d, J 7, H-4′ and H-5′).

### EXAMPLE SYNTHESIS 2

### Preparation of 2S,4RS-2-(9H-Fluoren-9-ylmethoxycarbonylamino)-4,5-dimethyl-hexanoic acid (74)

Optically pure 2S,4S-2-tert-Butoxycarbonylamino-4,5-dimethyl-hex-5-enoic acid methyl ester (69) and 2S,4R-2-tert-Butoxy-carbonylamino-4,5-dimethyl-hex-5-enoic acid methyl ester (70) were obtained directly after zinc coupling reaction by flash chromatography.

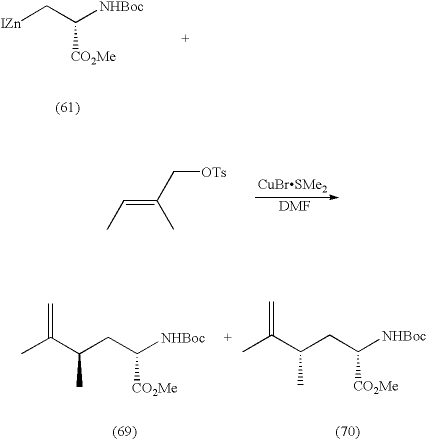

### (a) 2S,4S-2-tert-Butoxycarbonylamino-4,5-dimethyl-hex-5-enoic acid methyl ester (69) and 2S,4R-2-tert-butoxy-carbonylamino-4,5-dimethyl-hex-5-enoic acid methyl ester (70)

Following the general procedure for zinc coupling reactions, toluene-4-sulfonic acid (E)-2-methyl-but-2-enyl ester (1.45 mL, 1.0 mmol) was coupled to compound (61) (247 mg, 0.75 mmol) in the presence of CuBr.SMe₂ (20 mg, 0.10 mmol) to give a residue which was purified by flash chromatography over silica gel eluting with EtOAc/40:60 petroleum ether (1:9, v/v) to give two diastereoisomers. Analytical HPLC Rt=22.49 mins (60%) and Rt=22.52 mins (40%). Fractions of the first eluted component were pooled to give one of the diastereoisomers obtained as a colourless oil, yield 36 mg, 18%. Next a mixture of the diastereomers as a colourless oil, yield 75 mg, 37% was obtained. Pure fractions containing the later eluted component were pooled to give the other diastereoisomer as a colourless oil, yield 19 mg, 9%. (The stereochemistry at the 4 position was not investigated). Spectral data obtained for the fast running diastereomer: Electrospray-MS m/z 272 (MH⁺); [α]_{D}²⁰+12.3 (c 1.06 in CHCl₃); IR (cap. film)/cm⁻¹ 3382 (s), 3070 (m), 2966 (s), 1746 (s), 1716 (s), 1616 (w), 1507 (s), 886 (m)

δ_{H} (500 MHz, CDCl₃) 1.06 (3H, d, J 7, CH₃CH), 1.45 (9H, s, C(CH₃)₃), 1.58 (1H, m, CH₃CH), 1.68 (3H, s, CH₃C═CH₂), 1.85 (1H, m, CH_{2A}CH), 1.97 (1H, m, CH_{2B}CH), 3.73 (3H, s, OCH₃), 4.29 (1H, m, NHCHCO₂CH₃), 4.72 (1H, s, CH_{2A}═CH), 4.95 (1H, d, J 1.5, CH_{2B}═CH) and 5.04 (1H, d, J7, NH) δ_{C} (125 MHz, CDCl₃) 18.61 (CH₃C═CH₂), 21.64 (CH₃CH), 28.32 (C(CH₃)₃), 30.79 (CH₃CHCH₂), 38.06 (CH₂CHNH), 52.00 (NHCHCO₂CH₃), 52.22 (OCH₃), 79.53 (C(CH₃)₃), 110.19 (CH₂═C(CH₃)), 144.62 (CH₂═C(CH₃)), 155.18 (OCONH) and 173.30 (CO₂CH₃).

Spectral data obtained for the slow running diastereoismer: Electrospray-MS m/z 272 (MH⁺); [α]_{D}²⁰+16.0 (c 0.60 in CHCl₃); IR (cap. film)/cm⁻¹ 3369 (s), 3073 (m), 2969 (s), 1747 (s), 1717 (s), 1617 (w), 1517 (s), 893 (m)

δ_{H} (500 MHz, CDCl₃) 1.04 (3H, d, J 7, CH₃CH), 1.44 (9H, s, C(CH₃)₃), 1.55 (1H, m, CH₃CH), 1.67 (3H, s, CH₃C═CH₂), 1.91 (1H, m, CH_{2A}CH), 2.37 (1H, m, CH_{2B}CH), 3.73 (3H, s, OCH₃), 4.26 (1H, m, NHCHCO₂CH₃), 4.75 (1H, d, J 1.5, CH_{2A}═CH), 4.79 (1H, d, J 1.5, CH_{2B}═CH) and 5.46 (1H, d, J 6.1, NH) δ_{C} (125 MHz, CDCl₃) 18.51 (CH₃C═CH₂), 20.14 (CH₃CH), 28.31 (C(CH₃)₃), 30.55 (CH₃CHCH₂), 37.64 (CH₂CHNH), 52.17 (NHCHCO₂CH₃), 52.22 (OCH₃), 79.74 (C(CH₃)₃), 111.27 (CH₂═C(CH₃)), 147.94 (CH₂═C(CH₃)), 155.36 (OCONH) and 173.83 (CO₂CH₃).

These diastereoisomers were not separated routinely and used as a mixture in subsequent reactions.

### (b) 2S,4RS-2-tert-Butoxycarbonylamino-4,5-dimethyl-hexanoic acid methyl ester (71)

Following the general procedure for alkene hydrogenation, compounds (69) and compound (70) (130 mg, 0.48 mmol) yielded a mixture of two diastereoisomers (71) which were not separated, obtained as a colourless oil, yield 128 mg, 98%. Analytical HPLC Rt 22.49 mins, electrospray-MS m/z 274 (MH⁺).

### (c) 2S,4RS-2-tert-Butoxycarbonylamino-4,5-dimethyl-hexanoic acid (72)

Following the general procedure for methyl ester saponification, compounds (71) (128 mg, 0.47 mmol) gave a inseparable mixture of compounds (72) as a colourless oil, yield 106 mg, 87%. Electrospray-MS m/z 260 (MH⁺). Analytical HPLC Rt=20.65 mins (100%).

### (d) 2S,4RS-2-Amino-4,5-dimethyl-hexanoic acid trifluoroacetic acid salt (73)

Following the general procedure of N-Boc removal using TFA, compounds (72) (106 mg, 0.41 mmol) gave an inseparable mixture of compounds (73) as a solid, yield 107 mg, 96% and used directly in the subsequent reaction. Electrospray-MS m/z 160 (MH⁺).

### (e) 2S,4RS-2-(9H-Fluoren-9-ylmethoxycarbonylamino)-4,5-dimethyl-hexanoic acid (74)

Following the general procedure for Fmoc protection of an amine, compounds (73) (107 mg, 0.39 mmol) gave on purification by flash chromatography over silica gel eluting with CHCl₃/CH₃OH (100:0 to 95:5, v/v) 2S,4RS-2-(9H-fluoren-9-ylmethoxycarbonylamino)-4,5-dimethyl-hexanoic acid (74) as a solid, yield 60 mg, 40% as a mixture of two diastereoisomers. Analytical HPLC Rt=23.83 mins (40%) and Rt=24.06 mins (60%). First eluted diastereomer: Electrospray-MS m/z 382 (MH⁺). Later eluted diastereomer: Electrospray-MS m/z 382 (MH⁺).

### EXAMPLE SYNTHESIS 3

### Preparation of 2S,5RS-2-(9H-Fluoren-9-ylmethoxycarbonylamino)-5,6-dimethyl-heptanoic acid (80) and 2S-2-(9H-Fluoren-9-ylmethoxycarbonylamino)-4,4,5-trimethyl-hexanoic acid (84)

(S)-2-tert-butyloxycarbonylamino-5,6-dimethyl-hept-5-enoic methyl ester (75) and (S)-2-tert-butyloxycarbonylamino-4,4,5-trimethyl-hex-5-enoic methyl ester (76) were obtained directly after zinc coupling reaction by flash chromatography.

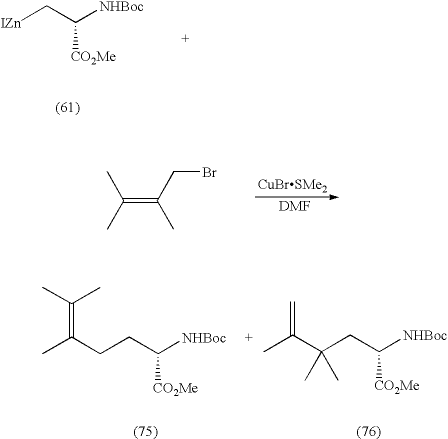

### (a) 2S-2-tert-Butyloxycarbonylamino-5,6-dimethyl-hept-5-enoic methyl ester (75)

### and 2S-2-tert-butyloxycarbonylamino-4,4,5-trimethyl-hex-5-enoic methyl ester (76)

Following the general procedure for zinc coupling reactions, 1-bromo-2,3-dimethylbut-2-ene (163 mg, 1.0 mmol) was coupled to compound (61) (247 mg, 0.75 mmol) in presence of CuBr.SMe₂ (20 mg, 0.10 mmol) to give a residue which on purification by flash chromatography over silica gel eluting with EtOAc/40:60 petroleum ether (1:9) gave two regioisomers. The first eluted component compound (75) as a colourless oil, yield 60 mg, 28% and the second eluted component was compound (76) as a colourless oil, yield 51 mg, 24%.

Spectral data obtained for compound (75); Electrospray-MS m/z 285 (MH⁺). Analytical HPLC Rt=22.85 mins (100%); HRMS C₁₅H₂₇NO₄ requires M, 285.1940, found: M⁺285.1954 (□−4.9 ppm); [α]_{D}²²+26.1 (c 1.01 in CH₂Cl₂); elemental analysis C₁₅H₂₇NO₄ (req) % C 63.1, % H 9.5, % N 4.9, (fnd) % C 62.4, % H 9.6, % N 5.3; IR (cap. film)/cm⁻¹ 3366 (s), 3154 (m), 2978 (s), 1744 (s), 1718 (s), 1506 (s), 1366 (s), 1164 (s)

δ_{H} (500 MHz, CDCl₃) 1.45 (9H, s, C(CH₃)₃), 1.62 (9H, m, (CH₃)₂═C(CH₂)), 1.87 (1H, m, CH_{2A}CH₂CH), 2.03 (1H, m, CH_{2B}CH₂CH), 2.09 (1H, dd, J 6, 10.5, CH₂CH_{2A}CH), 2.12 (1H, dd, J 6.5, 10.5, CH₂CH_{2B}CH), 3.74 (3H, s, OCH₃), 4.29 (1H, m, NHCHCO₂CH₃) and 5.02 (1H, d, J7, NH) δ_{C} (125 MHz, CDCl₃) 18.19 ((CH₃)₂C═C(CH₃)), 20.00 ((CH₃)_{2cis}C═C(CH₃)), 20.61 ((CH₃)₂ₜᵣₐₙₛC═C(CH₃)), 28.33 (C(CH₃)₃), 30.07 (CH₂CH₂CH), 30.92 (CH₂CH₂CH), 52.20 (NHCHCO₂CH₃), 53.47 (OCH₃), 80.00 (C(CH₃)₃), 95.90 ((CH₃)₂C═C(CH₃)), 96.49 ((CH₃)₂C═C(CH₃), 155.33 (OCONH) and 173.42 (CO₂CH₃).

Spectral data obtained for compound (76); Electrospray-MS m/z 285 (MH⁺). Analytical HPLC Rt=22.91 mins (100%); HRMS C₁₁H₁₉NO₄ requires M 229.1314, found: M⁺-C₄H₈ 229.1309 (□−2.2 ppm); [α]_{D}²³+4.8 (c 1.01 in CH₂Cl₂); elemental analysis C₁₅H₂₇NO₄ (req) % C 63.1, % H 9.5, % N 4.9, (fnd) % C 62.5, % H 9.5, % N; IR (cap. film)/cm⁻¹ 3368 (s), 3091 (m), 2934 (s), 1748 (s), 1717(s), 1516(s)

δ_{H}(500 MHz, CDCl₃) 1.10 (3H, s, (CH₃)_{2A}C), 1.12 (3H, s, (CH₃)_{2B}C), 1.43 (9H, s, C(CH₃)₃), 1.60 (1H, m, CH_{2A}CH), 1.74 (3H, s, CH₃C═CH₂), 1.92 (1H, dd, J 14.5, 4, CH_{2B}CH), 3.70 (3H, s, OCH₃), 4.24 (1H, m, NHCHCO₂CH₃), 4.79 (1H, s, CH_{2A}═C(CH₃)), 4.82 (1H, s, CH_{2B}═C(CH₃)) and 4.83 (1H, br d, J 11, NH) δ_{C}(125 MHz, CDCl₃) 19.38 (CH₃), 27.19 (CH₃), 27.61 (CH₃), 28.34 (C(CH₃)₃), 38.50 (CH₂CH), 38.95 ((CH₃)₂C), 51.34 (NHCHCO₂CH₃), 52.13 (OCH₃), 79.71 (C(CH₃)₃), 110.95 (CH₂═C(CH₃)), 150.62 (CH₂═C(CH₃)), 155.00 (OCONH) and 174.24 (CO₂CH₃).

### (b) 2S,5RS-2-tert-Butoxycarbonylamino-5,6-dimethyl-heptanoic acid methyl ester (77)

Following the general procedure for alkene hydrogenation, 2S-2-tert-butyloxycarbonylamino-5,6-dimethyl-hept-5-enoic methyl ester (75) (60 mg, 0.21 mmol) yielded compound (77) as a colourless oil, yield 54 mg, 89%. Electrospray-MS m/z 288 (MH⁺). Analytical HPLC Rt=24.06 mins (100%).

### (c) 2S,5RS-2-tert-Butoxycarbonylamino-5,6-dimethyl-heptanoic acid (78)

Following the general procedure for methyl ester saponification, compounds (77) (54 mg, 0.19 mmol) gave compounds (78) as a colourless oil, yield 54 mg, 100%. Electrospray-MS m/z 274 (MH⁺). Analytical HPLC Rt=21.44 mins (100%).

### (d) 2S,5RS-2-Amino-5,6-dimethyl-heptanoic acid hydrochloride salt (79)

Following the general procedure of N-Boc removal using 4M HCl in dioxane, compounds (78) (54 mg, 0.20 mmol) gave compounds (79) as a solid, yield 40 mg, 97%. Electrospray-MS m/z 174 (MH⁺).

### (e) 2S,5RS-2-(9H-Fluoren-9-ylmethoxycarbonylamino)-5,6-dimethyl-heptanoic acid (80)

Following the general procedure for Fmoc protection of an amine, compounds (79) (40 mg, 0.19 mmol) gave on purification by flash chromatography over silica gel eluting with CHCl₃/CH₃OH (100:0 to 95:5, v/v) 2S,5RS-2-(9H-fluoren-9-ylmethoxycarbonylamino)-5,6-dimethyl-heptanoic acid (80) as a solid, yield 27 mg, 36%. Electrospray-MS m/z 395 (MH⁺). Analytical HPLC Rt=24.52 mins (100%), HRMS C₂₄H₂₉O₄NNa requires M 418.1994, found: MNa⁺, 418.1993. (□−0.38 ppm)

δ_{H} (500 MHz; CDCl₃) 0.73 (6H, m, (CH₃)₂CH), 0.82 (3H, d, J 6.5, (CH₃)₂CHCH(CH₃)), 1.23 (1H, m, (CH₃)₂CHCH(CH₃)CH_{2A}), 1.39 (1H, m, (CH₃)₂CHCH(CH₃)CH_{2B}), 1.55 (2H, m, (CH₃)₂CHCH(CH₃) and (CH₃)₂CHCH(CH₃)CH₂CH_{2A}), 1.63 (1H, m, (CH₃)₂CHCH(CH₃)), 1.90 (1H, m, (CH₃)₂CHCH(CH₃)CH₂CH_{2B}), 4.18 (1H, t, J 6.5, CH-Fmoc), 4.40 (3H, m, NHCHCO₂H and CH₂O), 5.30 (1H, br d, J 8, NH-Fmoc), 7.27 (2H, m, H-2′ and H-7′), 7.37 (2H, m, H-3′ and H-6′), 7.56(2H, m, H-1′ and H-8′) and 7.75 (2H, d, J 7, H-4′ and H-5′)

δ_{C} (125 MHz; CDCl₃) 14.91 (CH₃)₂CHCH(CH₃)), 17.49 and 17.73 ((CH₃)_{2A}CH), 19.93 and 20.05 ((CH₃)_{2B}CH), 28.08 ((CH₃)₂CH), 29.26 and 29.44 ((CH₃)₂CHCH(CH₃)CH₂CH₂), 30.04 and 30.17 ((CH₃)₂CHCH(CH₃)CH₂CH₂), 31.38 and 31.68 ((CH₃)₂CHCH(CH₃)), 37.89 and 38.07 (NHCHCO₂H), 46.88 (CH-1′), 66.84 (CH₂O), 119.72 (CH-5′ and CH-10′), 124.80 (CH-4′ and CH-11′), 126.81 (CH-6′ and CH-9′), 127.46 (CH-3′ and CH-12′), 141.05 (C-7′ and C-8′), 143.47 (C-2′ and C-13′) and 155.89 (OCONH). The quaternary signal for the carboxylic acid was not observed.

### (f) 2S-2-tert-Butoxycarbonylamino-4,4,5-trimethyl-hexanoic acid methyl ester (81)

Following the general procedure for alkene hydrogenation, 2S-2-tert-butyloxycarbonylamino-4,4,5-trimethyl-hex-5-enoic methyl ester (76) (51 mg, 0.18 mmol) yielded compound (81) as a colourless oil, yield 46 mg, 90%. Electrospray-MS m/z 288 (MH⁺). Analytical HPLC Rt=22.91 mins (100%).

### (g) 2S-2-tert-Butoxycarbonylamino-4,4,5-trimethyl-hexanoic acid (82)

Following the general procedure for methyl ester saponification, compound (81) (46 mg, 0.16 mmol) gave compound (82) as a colourless oil, yield 44 mg, 100%. Electrospray-MS m/z 274 (MH⁺).

### (h) 2S-2-Amino-4,4,5-trimethyl-hexanoic acid hydrochloride salt (83)

Following the general procedure of N-Boc removal using 4M HCl in dioxane, compound (82) (44 mg, 0.16 mmol) gave compound (83) as a solid, yield 33 mg, 99%. Electrospray-MS m/z 174 (MH⁺).

### (i) 2S-2-(9H-Fluoren-9-ylmethoxycarbonylamino)-4,4,5-trimethyl-hexanoic acid (84)

Following the general procedure for Fmoc protection of an amine, compound (83) (33 mg, 0.16 mmol) gave on purification by flash chromatography over silica gel eluting with CHCl₃/CH₃OH (100:0 to 95:5, v/v) 2S-2-(9H-fluoren-9-ylmethoxycarbonylamino)-4,4,5-trimethyl-hexanoic acid (84) as a solid, yield 20 mg, 32%. Electrospray-MS m/z 396 (MH⁺). Analytical HPLC Rt=24.28 mins (100%), HRMS C₂₄H₂₉O₄NNa requires M 418.1994, found: MNa⁺, 418.1993. (□−0.38 ppm)

δ_{H} (500 MHz; CDCl₃) 0.93 (9H, m, (CH₃)₂CHC(CH₃)_{2A}), 0.98 (3H, s, (CH₃)₂CHC(CH₃)_{2B}), 1.48 (1H, dd, J 14, 10, (CH₃)₂CHC(CH₃)₂CH_{2A}), 1.57 (1H, m, (CH₃)₂CH), 1.91 (1H, d, J 14, (CH₃)₂CHC(CH₃)₂CH_{2B}), 4.21 (1H, t, J 6.5, CH-Fmoc), 4.40 (3H, m, NHCHCO₂H and CH₂O), 5.10 (1H, br d, J 7.5, NH-Fmoc), 7.27 (2H, m, H-2′ and H-7′), 7.36 (2H, m, H-3′ and H-6′), 7.57 (2H, m, H-1′ and H-8′) and 7.74 (2H, d, J 7, H-4′ and H-5′) δ_{C} (125 MHz; CDCl₃) 17.01 ((CH₃)_{2A}CH), 17.16 ((CH₃)_{2B}CH), 23.69 ((CH₃)₂CHC(CH₃)_{2A}), 24.27 ((CH₃)₂CHC(CH₃)_{2B}), 35.27 ((CH₃)₂CHC(CH₃)₂), 35.73 ((CH₃)₂CH), 41.88 ((CH₃)₂CHC(CH₃)₂CH₂), 46.93 (CH-1′), 54.20 (NHCHCO₂H), 66.79 (CH₂O), 119.70 (CH-5′ and CH-10′), 124.78 (CH-4′ and CH-11′), 126.79 (CH-6′ and CH-9′), 127.44 (CH-3′ and CH-12′), 141.05 (C-7′ and C-8′), 143.61 (C-2′ and C-13′) and 155.68 (OCONH). The quaternary signal for the carboxylic acid was not observed.

### General Solid Phase Procedures

Molecules were assembled using the furanone and pyranone building blocks and novel protected aminoacids described earlier, by solid phase procedures on Chiron multipins following the protocols detailed below.

### Preparation of Building Block-Linker Constructs

### General Method for the Synthesis of dihydro-3(2H)-furanone or pyranone—Linker Constructs—See Scheme 5 Above

Dihydro-3(2H)-furanone (18, 24-28), (1.0 eq) was dissolved in a mixture of ethanol/water (7:1 v/v, 10 mL per mmole compound) containing sodium acetate trihydrate (1.5 eq). 4-[[(hydrazinocarbonyl)amino]methyl]-cyclohexanecarboxylic acid trifluoro acetate (mw 329.3, 1.0 eq) (see Murphy, A. M., et al, J. Am. Chem. Soc, 114, 3156-3157, 1992) was added and the mixture heated under reflux for 2 hrs. The mixture was then cooled, poured into dichloromethane (100 mL per mmole compound) and water (100 mL) added. The organic layer was separated, backwashed with saturated brine (100 mL). The organic layer was dried (Na₂SO₄), filtered and evaporated in vacuo to yield a white solid. Yield 85-105% crude weight. Constructs (29-34) were used without further purification

### Preparation of Crown Assembly

The compounds were synthesised in parallel fashion using the appropriately loaded Fmoc-Building block-linker-DA/MDA derivatised macrocrowns (see above) loaded at approximately 3.5-9.1 μmoles per crown. Prior to synthesis each crown was connected to its respective stem and slotted into the 8×12 stem holder. Coupling of the amino acids employed standard Fmoc amino acid chemistry as described in ‘Solid Phase Peptide Synthesis’, E. Atherton and R. C. Sheppard, IRL Press Ltd, Oxford, UK, 1989.

### Removal of Nα-Fmoc Protection

A 250 mL solvent resistant bath is charged with 200 mL of a 20% piperidine/DMF solution. The multipin assembly is added and deprotection allowed to proceed for 30 minutes. The assembly is then removed and excess solvent removed by brief shaking. The assembly is then washed consecutively with (200 mL each), DMF (5 minutes) and MeOH (5 minutes, 2 minutes, 2 minutes) and left to air dry for 15 minutes.

### Quantitative UV Measurement of Fmoc Chromophore Release

A 1 cm path length UV cell is charged with 1.2 mL of a 20% piperidine/DMF solution and used to zero the absorbance of the UV spectrometer at a wavelength of 290 nm. A UV standard is then prepared consisting of 5.0 mg Fmoc-Asp(OBut)-Pepsyn KA (0.08 mmol/g) in 3.2 mL of a 20% piperidine/DMF solution. This standard gives Abs₂₉₀=0.55-0.65 (at room temperature). An aliquot of the multipin deprotection solution is then diluted as appropriate to give a theoretical Abs₂₉₀=0.6, and this value compared with the actual experimentally measured absorbance showing the efficiency of previous coupling reaction.

### Standard Coupling of Amino Acid Residues

Coupling reactions are performed by charging the appropriate wells of a polypropylene 96 well plate with the pattern of activated solutions required during a particular round of coupling. Macrocrown standard couplings were performed in DMF (500 μl).

### Coupling of an Amino-Acid Residue to Appropriate Well

Whilst the multipin assembly is drying, the appropriate N_{α}-Fmoc amino acid pfp esters (10 equivalents calculated from the loading of each crown) and HOBt (10 equivalents) required for the particular round of coupling are accurately weighed into suitable containers. Alternatively, the appropriate N_{α}-Fmoc amino acids (10 equivalents calculated from the loading of each crown), desired coupling agent e.g. HBTU (9.9 equivalents calculated from the loading of each crown) and activation e.g. HOBt (9.9 equivalents calculated from the loading of each crown), NMM (19.9 equivalents calculated from the loading of each crown) are accurately weighed into suitable containers. The protected and activated Fmoc amino acid derivatives are then dissolved in DMF (500 μl for each macrocrown e.g. for 20 macrocrowns, 20×10 eq.×7 μmoles of derivative would be dissolved in 10 mL DMF). The appropriate derivatives are then dispensed to the appropriate wells ready for commencement of the ‘coupling cycle’. As a standard, coupling reactions are allowed to proceed for 6 hours. The coupled assembly was then washed as detailed below.

### Washing Following Coupling

If a 20% piperidine/DMF deprotection is to immediately follow the coupling cycle, then the multipin assembly is briefly shaken to remove excess solvent washed consecutively with (200 mL each), MeOH (5 minutes) and DMF (5 minutes) and de-protected. If the multipin assembly is to be stored or reacted further, then a full washing cycle consisting brief shaking then consecutive washes with (200 mL each), DMF (5 minutes) and MeOH (5 minutes, 2 minutes, 2 minutes) is performed.

### Addition of Capping Group

Whilst the multipin assembly is drying, the appropriate acid capping group (10 equivalents calculated from the loading of each crown), desired coupling agent e.g. HBTU (9.9 equivalents calculated from the loading of each crown) and activation e.g. HOBt (9.9 equivalents calculated from the loading of each crown), NMM (19.9 equivalents calculated from the loading of each crown) are accurately weighed into suitable containers. The acid derivatives/coupling agents are then dissolved in DMF (500 μl for each macrocrown e.g. for 20 macrocrowns, 20×10 eq. of derivative would be dissolved in 10 mL DMF) and left to activate for 5 minutes. The appropriate derivatives are then dispensed to the appropriate wells ready for commencement of the ‘capping cycle’. As a standard, capping reactions are allowed to proceed for 18 hours overnight. The capped assembly was then washed as detailed above.

### Acidolytic Mediated Cleavage of Molecule-Pin Assembly

Acid mediated cleavage protocols are strictly performed in a fume hood. A polystyrene 96 well plate (1 mL/well) is labelled and weighed to the nearest mg. Appropriate wells are then charged with a trifluoroacetic acid/water (95:5, v/v, 600 μl) cleavage solution, in a pattern corresponding to that of the multipin assembly to be cleaved.

The multipin assembly is added, the entire construct covered in tin foil and left for 2 hours. The multipin assembly in then added to another polystyrene 96 well plate (1 mL/well) containing trifluoroacetic acid/water (95:5, v/v, 600 □l) (as above) for 5 minutes.

### Work up of Cleaved Molecules

The primary polystyrene cleavage plate (2 hour cleavage) and the secondary polystyrene plate (5 minute wash) are then placed in the GeneVac evaporator and the solvents removed (minimum drying rate) for 90 minutes. The contents of the secondary polystyrene plate are transferred to their corresponding wells on the primary plate using an acetonitrile/water (50: 50 v/v/v) solution (3×150 μl) and the spent secondary plate discarded. Aliqouts (5-20 □L) are taken for analysis. The plate was covered in tin foil, pin-pricked over wells containing compounds, placed into the freezer for 1 hr, then lyophilised.

### Analysis and Purification of Molecules

The (5-20 □L) aliquots are analysed by analytical HPLC and electrospray-MS. In virtually all cases, crude purities are >90% by HPLC with the desired m/z. Sample were purified by semi-preparative reverse phase HPLC, using Vydac C₄.Appropriate fractions are combined and lyophilised in tared 10 mL glass vials, then re-weighed. Molecules were prepared on a 15-90 μmole scale, yielding 2.0-26.0 mg of purified products. The purity of each product was confirmed by analytical HPLC at >95% (215 nm UV detection) and gave the appropriate [MH]⁺ by electrospray mass spectrometry analysis.

### Loading of Macrocrowns with Constructs

### General method for the loading of multipins with Dihydro-3(2H)-Furanone—Linker Constructs (29-34)

Amino functionalised DA/MDA macrocrowns (ex Chiron Mimotopes, Australia, 9.1 μmole loading) or amino functionalised HEMA gears (ex Chiron Mimotopes, Australia, 1.3 μmole loading) were used for all loadings and subsequent solid phase syntheses.

Dihydro-3(2H)-Furanone—Linker Construct (29-34) (3 eq compared to total surface functionalisation of crowns/gears) was carboxyl activated with 2-(1H-benzotriazole-1-yl )-1,1,3,3-tetramethyluronium hexafluorophosphate (3 eq), 1-hydroxybenzotriazole (3 eq) and N-methylmorpholine (6 eq) in dimethylformamide (5 mL) for 5 mins. This mixture was added to the crowns/gears, additional DMF added to cover the reaction surface and the mixture left overnight.

Standard washing and Fmoc deprotection readings (see procedures above) indicated virtually quantitative loading.

Exemplar molecules prepared by the methods using the respective furanone, R3 amino acid and capping group in the method detailed above are shown in Table 1:

| Electrospray-MS | |
|---|---|
| m/z (MH⁺) | NAME |
| 385 | Benzofuran-2-carboxylic acid |
| | [1S-(2R-methyl-4-oxo-tetrahydro-furan- |
| | 3S-ylcarbamoyl)-cyclohexyl]-amide |
| 383 | Benzofuran-2-carboxylic acid |
| | [1-(2R-methyl-4-oxo-tetrahydro-furan-3S- |
| | ylcarbamoyl)-cyclohexyl]-amide |
| 371 | Benzofuran-2-carboxylic acid |
| | [2-cyclopropyl-1S-(2R-methyl-4-oxo-tetrahydro- |
| | furan-3S-ylcarbamoyl)-ethyl]-amide |
| 398 | N-[3-Methyl-1S-(2R-methyl-4-oxo-tetrahydro- |
| | furan-3S-ylcarbamoyl)-butyl]-4-pyrrol-1-yl-benzamide |
| 383 | Naphthalene-1-carboxylic acid |
| | [3-methyl-1S-(2R-methyl-4-oxo-tetrahydro- |
| | furan-3S-ylcarbamoyl)-butyl]-amide |
| 373 | Benzofuran-2-carboxylic acid |
| | [3-methyl-1S-(2R-methyl-4-oxo-tetrahydro-furan- |
| | 3S-ylcarbamoyl)-butyl]-amide |
| 389 | Benzo[b]thiophene-2-carboxylic acid |
| | [3-methyl-1S-(2R-methyl-4-oxo-tetrahydro-furan- |
| | 3S-ylcarbamoyl)-butyl]-amide |
| 403 | 5-Methoxy-benzofuran-2-carboxylic acid |
| | [3-methyl-1S-(2R-methyl-4-oxo-tetrahydro-furan-3S- |
| | ylcarbamoyl)-butyl]-amide |
| 401 | 5-Methoxy-benzofuran-2-carboxylic acid |
| | [3-methyl-1S-(2R-methyl-4-oxo-tetrahydro-furan-3- |
| | ylcarbamoyl)-but-3S-enyl]-amide |
| 390 | 4-Acetylamino-N-[3-methyl-1S-(2R-methyl-4- |
| | oxo-tetrahydro-furan-3S-ylcarbamoyl)- |
| | butyl]-benzamide |
| 448 | 4-Hydroxy-N-[3-methyl-1S-(2R-methyl-4-oxo- |
| | tetrahydro-furan-3S-ylcarbamoyl)-butyl]-3-morpholin- |
| | 4-ylmethyl-benzamide |
| 446 | 4-Hydroxy-N-[3-methyl-1S-(2R-methyl-4-oxo- |
| | tetrahydro-furan-3S-ylcarbamoyl)-but-3-enyl]-3- |
| | morpholin-4-ylmethyl-benzamide |
| 409 | Biphenyl-4-carboxylic acid |
| | [3-methyl-1S-(2R-methyl-4-oxo-tetrahydro- |
| | furan-3S-ylcarbamoyl)-butyl]-amide |
| 389 | 4-tert-Butyl-N-[3-methyl-1S-(2R-methyl-4- |
| | oxo-tetrahydro-furan-3S-ylcarbamoyl)- |
| | butyl]-benzamide |
| 387 | 4-tert-Butyl-N-[3-methyl-1S-(2R-methyl-4- |
| | oxo-tetrahydro-furan-3S-ylcarbamoyl)-but-3- |
| | enyl]-benzamide |
| 390 | 4-Guanidino-N-[3-methyl-1S-(2R-methyl-4-oxo- |
| | tetrahydro-furan-3S-ylcarbamoyl)-butyl]-benzamide |
| 388 | 4-Guanidino-N-[3-methyl-1S-(2R-methyl-4-oxo- |
| | tetrahydro-furan-3S-ylcarbamoyl)-but-3- |
| | enyl]-benzamide |
| 502 | 5-(2-Morpholin-4-yl-ethoxy)-benzofuran-2-carboxylic |
| | acid [3-methyl-1S-(2R-methyl-4-oxo-tetrahydro-furan- |
| | 3S-ylcarbamoyl)-butyl]-amide |
| 500 | 5-(2-Morpholin-4-yl-ethoxy)-benzofuran-2-carboxylic |
| | acid [3-methyl-1S-(2R-methyl-4-oxo-tetrahydro-furan |
| | -3S-ylcarbamoyl)-but-3-enyl]-amide |

Additional compounds of the invention were prepared as follows:

### i) Preparation of N-[3-Methyl-1-(2-methyl-4-oxo-tetrahydro-furan-3-ylcarbamoyl)-butyl]-4-pyrrolidin-1-yl-benzamide

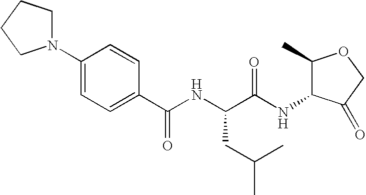

Following the procedure of Example 1 step d) except substituting “4-pyrrolidin-1-yl-benzoic acid” for the model carboxylic acid. These acids were prepared by employing two reactions: the initial esters were prepared using Buchwald type chemistry and subsequent standard hydrolysis of the esters provided the required acids.

### a. 4-Pyrrolidin-1-yl-benzoic acid methyl ester

An oven-dried reaction tube was charged with cesium carbonate (2.12 g, 6.51 mmol) that had been finely ground with a pestle and mortar under an atmosphere of argon. Tris(dibenzylideneacetone)dipalladium(0) (42.5 mg, 1.5 mol %) and (S)-(−)-2,2′-bis(diphenylphosphino)-1,1′-binaphthyl (43.4 mg, 1.5 mol %) were added and the tube charged with argon. Pyrrolidine (0.39 g, 5.58 mmol), methyl-4-bromobenzoate (1.00 g, 4.65 mmol) and toluene (10 ml) were added and the mixture heated to 100° C. with vigorous stirring until the starting material had been consumed as judged by hplc. The mixture was cooled to room temperature, diluted with ether (20 ml), filtered and concentrated. Purification by column chromatography gave the title compound (0.80 g, 84%) as a solid. MS (M+H+): 206.

### b. 4-Pyrrolidin-1-yl-benzoic acid salt

4-Pyrrolidin-1-yl-benzoic acid methyl ester (200 mg, 0.98 mmol) was dissolved in methanol (4 ml) and sodium hydroxide (39 mg, 0.98 mmol) in water (2 ml) was added. The mixture was heated to 60° C. until the starting material had been consumed as judged by hplc. The mixture was cooled to room temperature, diluted with water (20 ml), filtered and freeze dried to give the title compound (0.200 g, 97%) as a solid. MS (M+H+): 192.

An alternative procedure is described below:

4-Pyrrolidin-1-yl-benzoic acid methyl ester (200 mg, 0.98 mmol) was dissolved in concentrated hydrochloric acid: water (1:1) (4 ml). The mixture was heated at reflux until the starting material had been consumed as judged by hplc. The mixture was cooled to room temperature, diluted with water (10 ml), filtered and freeze dried to give the title compound (0.190 g, 97%) as a solid. MS (M+H+): 192.

### Preparation of N-[3-Methyl-1-(2-methyl-4-oxo-tetrahydro-furan-3-ylcarbamoyl)-butyl]-4-piperidin-1-yl-benzamide

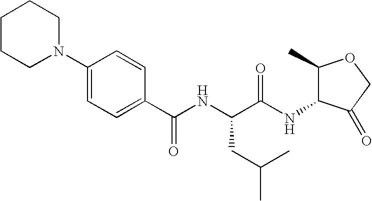

Following the procedure of Example 1 step d) except substituting “4-piperidin-1-yl-benzoic acid”

### 4-Piperidin-1-yl-benzoic acid methyl ester

Following the procedure above except substituting “piperidine” for “pyrrolidine” gave the title compound: MS (M+H+): 220.

### a. 4-Piperidin-1-yl-benzoic acid salt

Following the procedure above except substituting “4-piperidin-1-yl-benzoic acid methyl ester” for “4-pyrrolidin-1-yl-benzoic acid methyl ester” gave the title compound: MS (M+H+): 206.

### Preparation of N-[3-Methyl-1-(2-methyl-4-oxo-tetrahydro-furan-3-ylcarbamoyl)-butyl]-4-morpholin-4-yl-benzamide

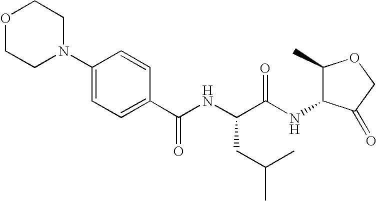

Following the procedure of Example 1 step d) except substituting “4-morpholin-4-yl-benzoic acid”

### 4-Morpholin-4-yl-benzoic acid methyl ester

Following the procedure above except substituting “morpholine” for “pyrrolidine” gave the title compound: MS (M+H+): 222.

### a. 4-Morpholin-4-yl-benzoic acid salt

Following the procedure above except substituting “4-morpholin-4-yl-benzoic acid methyl ester” for “4-pyrrolidin-1-yl-benzoic acid methyl ester” gave the title compound: MS (M+H+): 208.

### Preparation of N-[3-Methyl-1-(2-methyl-4-oxo-tetrahydro-furan-3-ylcarbamoyl)-butyl]-4-(4-methyl-piperazin-1-yl)-benzamide

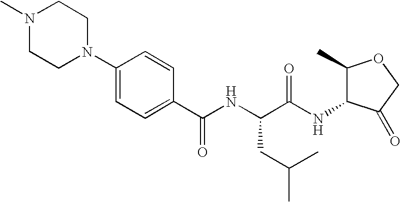

Following the procedure of Example 1 step d) except substituting “4-methyl-piperazin-1-yl-benzoic acid” for

### 4-Methyl-piperazin-1-yl-benzoic acid methyl ester

Following the procedure above except substituting “4-methyl-piperazine” for “pyrrolidine” gave the title compound: MS (M+H+): 235.

### a. 4-Methyl-piperazin-1-yl-benzoic acid salt

Following the procedure above except substituting “4-methyl-piperazin-1-yl-benzoic acid methyl ester” for “4-pyrrolidin-1-yl-benzoic acid methyl ester” gave the title compound: MS (M+H+): 221.

### Preparation of N-[3-Methyl-1-(2-methyl-4-oxo-tetrahydro-furan-3-ylcarbamoyl)-butyl]-4-(2-morpholin-4-yl-ethylamino)-benzamide

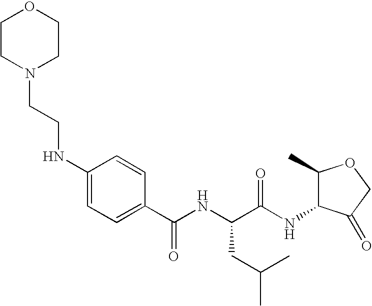

Following the procedure of Example 1 step d) except substituting “4-(2-morpholin-4-yl-ethylamino)-benzoic acid”

### a. 4-(2-Morpholin-4-yl-ethylamino)-benzoic acid methyl ester

Following the procedure above except substituting “2-morpholin-4-yl-ethylamine” for “pyrrolidine” gave the title compound: MS (M+H+): 265.

### b. 4-(2-Morpholin-4-yl-ethylamino)-benzoic acid salt

Following the procedure above except substituting “4-(2-Morpholin-4-yl-ethylamino)-benzoic acid methyl ester” for “4-pyrrolidin-1-yl-benzoic acid methyl ester” gave the title compound: MS (M+H+): 251.

### Preparation of N-[3-Methyl-1-(2-methyl-4-oxo-tetrahydro-furan-3-ylcarbamoyl)-butyl]-4-piperazin-1-yl-benzamide

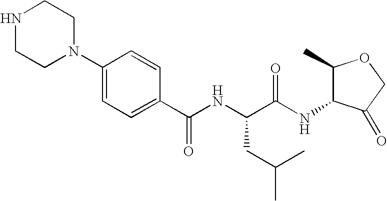

Following the procedure of Example 1 step d) except substituting “4-(4-carboxy-phenyl)-piperazine-1-carboxylic acid tert-butyl ester”.

### a. 4-(4-Methoxycarbonyl-phenyl)-piperazine-1-carboxylic acid tert-butyl ester

Following the procedure above except substituting “piperazine-1-carboxylic acid tert-butyl ester” for “pyrrolidine” gave the title compound: MS (M+H+): 321.

### b. 4-(4-Carboxy-phenyl)-piperazine-1-carboxylic acid tert-butyl ester salt

Following the procedure above except substituting “4-(4-methoxycarbonyl-phenyl)-piperazine-1-carboxylic acid tert-butyl ester” for “4-pyrrolidin-1-yl-benzoic acid methyl ester” gave the title compound: MS (M+H+): 307.

### Preparation of N-[3-Methyl-1-(2-methyl-4-oxo-tetrahydro-furan-3-ylcarbamoyl)-butyl]-3-pyrrolidin-1-yl-benzamide

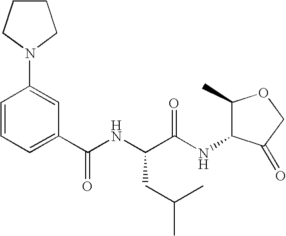

Following the procedure of Example 1 step d) except substituting “3-pyrrolidin-1-yl-benzoic acid” for “2-benzofuran-carboxylic acid.”

### a. 3-Pyrrolidin-1-yl-benzoic acid methyl ester

Following the procedure above except substituting “methyl-3-bromobenzoate” for “methyl-4-bromobenzoate” gave the title compound: MS (M+H+): 206.

### b. 3-Pyrrolidin-1-yl-benzoic acid salt

Following the procedure above except substituting “3-pyrrolidin-1-yl-benzoic acid methyl ester” for “4-pyrrolidin-1-yl-benzoic acid methyl ester” gave the title compound: MS (M+H+): 192.

### Preparation of N-[3-Methyl-1-(2-methyl-4-oxo-tetrahydro-furan-3-ylcarbamoyl)-butyl]-3-piperidin-1-yl-benzamide

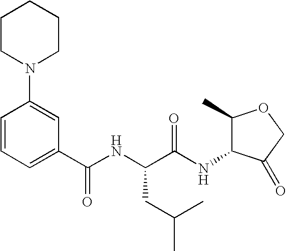

Following the procedure of Example 1 step d) except substituting “3-piperidin-1-yl-benzoic acid” for “2-benzofuran-carboxylic acid.”

### a. 3-Piperidin-1-yl-benzoic acid methyl ester

Following the procedure above except substituting “piperidine” for “pyrrolidine” and “methyl-3-bromobenzoate” for “methyl-4-bromobenzoate” gave the title compound: MS (M+H+): 220.

### b. 3-Piperidin-1-yl-benzoic acid salt

Following the procedure above except substituting “3-piperidin-1-yl-benzoic acid methyl ester” for “4-pyrrolidin-1-yl-benzoic acid methyl ester” gave the title compound: MS (M+H+): 206.

### Preparation of N-[3-Methyl-1-(2-methyl-4-oxo-tetrahydro-furan-3-ylcarbamoyl)-butyl]-3-morpholin-4-yl-benzamide

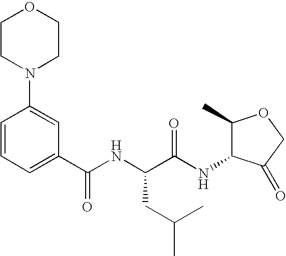

Following the procedure of Example 1 step d) except substituting “3-morpholin-4-yl-benzoic acid”.

### a. 3-Morpholin-4-yl-benzoic acid methyl ester

Following the procedure above except substituting “morpholine” for “pyrrolidine” and “methyl-3-bromobenzoate” for “methyl-4-bromobenzoate” gave the title compound: MS (M+H+): 222.

### b. 3-Morpholin-4-yl-benzoic acid salt

Following the procedure above except substituting “3-morpholin-4-yl-benzoic acid methyl ester” for “3-pyrrolidin-1-yl-benzoic acid methyl ester” gave the title compound: MS (M+H+): 208.

### Preparation of N-[3-Methyl-1-(2-methyl-4-oxo-tetrahydro-furan-3-ylcarbamoyl)-butyl]-3-(4-methyl-piperazin-1-yl)-benzamide

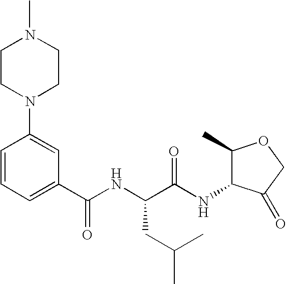

Following the procedure of Example 1 step d) except substituting “3-methyl-piperazin-1-yl-benzoic acid” for “2-benzofuran-carboxylic acid.”

### a. 3-Methyl-piperazin-1-yl-benzoic acid methyl ester

Following the procedure above except substituting “4-methyl-piperazine” for “pyrrolidine” and “methyl-3-bromobenzoate” for “methyl-4-bromobenzoate” gave the title compound: MS (M+H+): 235.

### b. 3-Methyl-piperazin-1-yl-benzoic acid salt

Following the procedure above except substituting “3-methyl-piperazin-1-yl-benzoic acid methyl ester” for “4-pyrrolidin-1-yl-benzoic acid methyl ester” gave the title compound: MS (M+H+): 221.

### Preparation of N-[3-Methyl-1-(2-methyl-4-oxo-tetrahydro-furan-3-ylcarbamoyl)-butyl]-3-(2-morpholin-4-yl-ethylamino)-benzamide

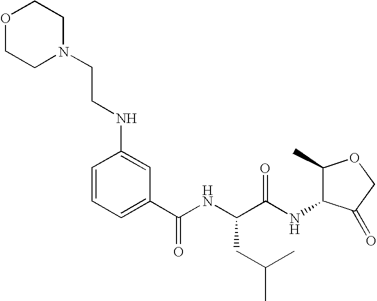

Following the procedure of Example 1 step d) except substituting “3-(2-morpholin-4-yl-ethylamino)-benzoic acid” for “2-benzofuran-carboxylic acid.”

### a. 3-(2-Morpholin-4-yl-ethylamino)-benzoic acid methyl ester

Following the procedure above except substituting “2-morpholin-4-yl-ethylamine” for “pyrrolidine” and “methyl-3-bromobenzoate” for “methyl-4-bromobenzoate” gave the title compound: MS (M+H+): 265.

### b. 3-(2-Morpholino-4-yl-ethylamino)-benzoic acid salt

Following the procedure above except substituting “3-(2-Morpholin-4-yl-ethylamino)-benzoic acid methyl ester” for “4-pyrrolidin-1-yl-benzoic acid methyl ester” gave the title compound: MS (M+H+): 251.

### Preparation of N-[3-Methyl-1-(2-methyl-4-oxo-tetrahydro-furan-3-ylcarbamoyl)-butyl]-3-piperazin-1-yl-benzamide

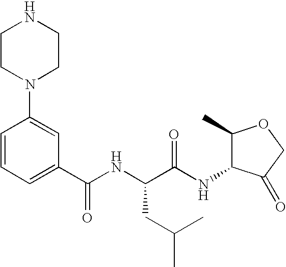

Following the procedure of Example 1 step d) except substituting “4-(3-carboxy-phenyl)-piparazine-1-carboxylic acid tert-butyl ester”

### b. 3-(4-Methoxycarbonyl-phenyl)-piperazine-1-carboxylic acid tert-butyl ester

Following the procedure above except substituting “piperazine-1-carboxylic acid tert-butyl ester” for “pyrrolidine” and “methyl-3-bromobenzoate” for “methyl-4-bromobenzoate” gave the title compound: MS (M+H+): 321.

### b. 4-(4-Carboxy-phenyl)-piperazine-1-carboxylic acid tert-butyl ester salt

Following the procedure above except substituting “4-(4-methoxycarbonyl-phenyl)-piperazine-1-carboxylic acid tert-butyl ester” for “4-pyrrolidin-1-yl-benzoic acid methyl ester” gave the title compound: MS (M+H+): 307.

### BIOLOGICAL EXAMPLES

### Determination of Cathepsin K Proteolytic Catalytic Activity

Convenient assays for cathepsin K are carried out using human recombinant enzyme. Standard assay conditions for the determination of kinetic constants used a fluorogenic peptide substrate, typically H-D-Ala-Leu-Lys-AMC, and were determined in either 100 mM Mes/Tris, pH 7.0 containing 1 mM EDTA and 10 mM 2-mercaptoethanol or 100 mM Na acetate, pH 5.5 containing 5 mM EDTA and 20 mM cysteine. The enzyme concentration used was 5 nM. The stock substrate solution was prepared at 10 mM in DMSO. Screens were carried out at a fixed substrate concentration of 60 μM and detailed kinetic studies with doubling dilutions of substrate from 250 μM. The total DMSO concentration in the assay was kept below 3%. All assays were conducted at ambient temperature. Product fluorescence (excitation at 390 nm, emission at 460 nm) was monitored with a Labsystems Fluoroskan Ascent fluorescent plate reader. Product progress curves were generated over 15 minutes following generation of AMC product.

### Inhibition Studies

Potential inhibitors are screened using the above assay with variable concentrations of test compound. Reactions were initiated by addition of enzyme to buffered solutions of substrate and inhibitor. Kᵢ values were calculated according to equation 1

$${v}_{0} = \frac{VS}{{K}_{M} ( 1 + \frac{I}{{K}_{i}} ) + S} \qquad ( 1 )$$

where v₀ is the velocity of the reaction, V is the maximal velocity, S is the concentration of substrate with Michaelis constant of K_{M}, and I is the concentration of inhibitor.

In this assay the compounds depicted in Table I have a Kᵢ values at pH 7 in the range 10 nM to 250 nM and are thus have utility in the treatment or prophylaxis of disorders in which cathepsin K is implicated, such as osteoporosis, gingival diseases such as gingivitis and periodontitis, Paget's disease, hypercalcaemia of malignancy, metabolic bone disease, osteoarthritis, rheumatoid arthritis, and metastatic neoplastias.

### Cloning and Expression of Falcipain II

### Generation of Falcipain 2

### Cloning

The deoxyoligonucleotide primers:

| | |
|---|---|
| 5′CGCGGATCCGCCACCATGGAATTAAACAGATTTGCCGAT-3′ | (SEQ ID NO.: 1) |
| and | |
| 5′CGCGTCGACTTAATGATGATGATGATGATGTTCAATTAATGGAATGAAT | (SEQ ID NO.: 2) |
| GCATCAGT-3′ were designed based on sequences deposited at the Sanger | |
| Centre, Cambridge, UK | |

(http://www.sanger.ac.uk/Projects/P_falciparum/blast_server.shtml). These primers were designed to amplify a portion of the cDNA sequence of the cysteinyl proteinase now known as Falcipain 2 and to include relevant terminal cloning enzymes sites and a carboxy-terminal hexahistidine coding sequence immediately upstream of the stop codon.

Polymerase chain reaction was performed with the above primers and Plasmodium falciparum phage library DNA as a template using the following conditions; 94° C. for 2 minutes then 35 cycles of 94° C. for 10 seconds, 50° C. for 1 minute, and 60° C. for 2 minutes, this was followed by a 60° C. 5 minute incubation. The 880 bp PCR amplicon was purified and phosphorylated using T4 polynucleotide kinase. This DNA was then ligated into EcoRV cleaved, dephosphorylated Bluescript II cloning vector and transformed into DH5 alpha E.coli. The DNA sequence of the plasmid inserts in isolated recombinant E.coli clones were determined using an Amersham Megabace sequencing instrument. To create an authentic ORF a three-way ligation was conducted bringing together the N-terminus of truncated falcipain-2 (NcoI/NdeI), the C-terminus of falcipain-2 (NdeI/BamH1) and the vector pQE-60 (NcoI/BamHI).

Nucleotide Sequence of TF2.10 (SEQ ID NO.: 3):
CCATGGAATTAAACAGATTTGCCGATTTAACTTATCATGAATTTAAAAA
CAAATATCTTAGTTTAAGATCTTCAAAACCATTAAAGAATTCTAAATAT
TTATTAGATCAAATGAATTATGAAGAAGTTATAAAAAAATATAGAGGAG
AAGAAAATTTCGATCATGCAGCTTACGACTGGAGATTACACAGTGGTGT
AACACCTGTAAAGGATCAAAAAAATTGTGGATCTTGCTGGGCCTTTAGT
AGTATAGGTTCCGTAGAATCACAATATGCTATCAGAAAAAATAAATTAA
TAACCTTAAGTGAACAAGAATTAGTAGATTGTTCATTTAAAAATTATGG
TTGTAATGGAGGTCTCATTAATAATGCCTTTGAGGATATGATTGAACTT
GGAGGTATATGTCCAGATGGTGATTATCCATATGTGAGTGATGCTCCAA
ATTTATGTAACATAGATAGATGTACTGAAAAATATGGAATCAAAAATTA
TTTATCCGTACCAGATAATAAATTAAAAGAAGCACTTAGATTCTTGGGA
CCTATTAGTATTAGTGTAGCCGTATCAGATGATTTTGCTTTTTACAAAG
AAGGTATTTTCGATGGAGAATGTGGTGATGAATTAAATCATGCCGTTAT
GCTTGTAGGTTTTGGTATGAAAGAAATTGTTAATCCATTAACCAAGAAA
GGAGAAAAACATTATTATTATATAATTAAGAACTCATGGGGACAACAAT
GGGGAGAAAGAGGTTTCATAAATATTGAAACAGATGAATCAGGATTAAT
GAGAAAATGTGGATTAGGTACTGATGCATTCATTCCATTAATTGAACAT
CATCATCATCATCATTAAGTCGACGCGATCGAATTCCTGCAGCCCGGGG
ATCC
Coding for the Protein Sequence (SEQ ID NO.: 4):
MELNRFADLTYHEFKNKYLSLRSSKPLKNSKYLLDQMNYEEVIKKYRGE
ENFDHAAYDWRLHSGVTPVKDQKNCGSCWAFSSIGSVESQYAIRKNKLI
TLSEQELVDCSFKNYGCNGGLINNAFEDMIELGGICPDGDYPYVSDAPN
LCNIDRCTEKYGIKNYLSVPDNKLKEALRFLGPISISVAVSDDFAFYKE
GIFDGECGDELNHAVMLVGFGMKEIVNPLTKKGEKHYYYIIKNSWGQQW
GERGFINIETDESGLMRKCGLGTDAFIPLIEHHHHHH.

The TF2.10 insert was excised from the pQE-60 vector using the restriction enzymes NcoI and BamHI, ligated into NcoI/BamHI cut expression vector pET-11D and transformed into DH5 alpha E.coli. The presence of a recombinant expression plasmid (pET-TF2.10) in an isolated E.coli colony was confirmed by restriction enzyme digest of plasmid DNA. BL21(DE3) E.coli were transformed with pET-TF2.10 and used for expression of the recombinant cysteinyl proteinase.

### Protein Expression

pET-TF2.10-Transformed BL21(DE3) E.coli (BLTF2.10) were grown up overnight at 200 rpm, 37° C. in Luria broth containing 100 μg/ml ampicillin. Fresh medium was then inoculated and grown to an OD_{600 nm} of 0.8 before protein expression was induced using 1 mM IPTG. Induction was performed for 3 hours at 200 rpm, 37° C. then the bacterial cells harvested by centrifugation and stored at −80° C. until protein purification performed.

### Protein Purification and Refolding

An E.coli cell pellet equivalent to 250 ml culture was lysed by resuspension in solubilisation buffer (6M guanidine hydrochloride, 20 mM Tris-HCl, 250 mM NaCl, 20 mM imidazole, pH8.0) for 30 minutes at room temperature. After centrifugation at 12000 g for 10 minutes at 4° C. the cleared lysate was applied to 1 ml nickel-NTA agarose, and agitated for 1 hour at room temperature.

### Protein Refolding Method 1

The protein bound to nickel-NTA was batch washed with 6M guanidine hydrochloride, 20 mM Tris-HCl, pH 8.o, 250 mM NaCl then 8M urea, Tris-HCl, pH 8.0, 500 mM NaCl then 8M urea, Tris-HCl, pH 8.0 including 30 mM imidazole and protein elution performed using 8M urea, Tris-HCl, pH 8.0 with 1 M imidazole. The eluted protein was then diluted 100 fold in refolding buffer (100 mM Tris-HCl, 1 mM EDTA, 20% glycerol, 250 mM L-arginine, 1 mM reduced glutathione, 0.1 mM oxidised glutatione, pH8.0) and left stirring overnight at 4° C. The protein could then be concentrated either by filter centrifugation or repurification using a nickel-agarose column (after dialysis to remove the EDTA).

### Protein Refolding Method 2

The protein bound to nickel-NTA was batch washed with 8M urea, Tris-HCl, 500 mM NaCl, pH 8.0 then 8M urea, Tris-HCl, pH 8.0 including 20 mM imidazole, then 2M urea, Tris-HCl, pH 8.0. The protein was then refolded on the column by the addition of 100 mM Tris-HCl, pH8.0, 250 mM L-arginine, 1 mM reduced glutathione, 0.1 mM oxidised glutatione with incubation at 4° C. and protein elution performed using, 100 mM Tris-HCl, pH 8.0 with 0.5 M imidazole.

Immediately active (mature) proteinase was obtained using protein refolding method 1 and concentrating the dilute refolded enzyme by filter centrifugation. This method, however, did result in a large degree of enzyme loss due to autoproteolysis. Both concentrating the protein refolded using method 1 by nickel column purification and using refolding method 2 resulted in greater recovery of the enzyme in its stable inactive pro-form. The pro-form could also be used to generate mature active falcipain 2, after incubation at 37° C.

The C-tagged construct outlined above enables rapid concentration and recovery after protein refolding and circumvents problems with autoproteolysis. Unlike the N-tagged constructs described in Shenai et al J Biol Chem 275 37 29000-29010m, the constructs described here can be refolded on the surface of an insoluble matrix, for instance bound to a purification column. Additionally the proregion can act as an enzyme inactivating sequence analogous to the native enzyme making work with the enzyme more predictable ie the stable inactive enzyme can be controllably activated when needed. An N-terminal tag would tend to prevent this normal functioning of the enzyme, as the proregion of the enzyme ought to be able to fold independently to direct the folding state of the mature enzyme domain. The tag described herein allows affinity purification to increase yields and enhance opportunities to isolate stable proforms of the enzyme.

Accordingly there is described an enzymatically active falcipain 2 construct comprising a covalently bonded C-terminal tag. The C-terminal tag may comprise polyhistidine, for example 4-8 residues, preferably 6. The construct described above has the tag at the C terminal of glutamic acid residue, but other constructs can shorten the enzyme by up to 10, for example 6-8 or 2-4 residues and retain a useful screening activity. Preferred constructs comprise the sequence enumerated above, optionally truncated at the C terminal as described herein.

### Determination of Falcipain 2 Proteolytic Catalytic Activity

Convenient assays for falcipain 2 are carried out using recombinant enzyme prepared above. Alternatively, falcipain is assayed as described in Sijwali et al Prot Exp Purif 22, 128-134 (2001). Standard assay conditions for the determination of kinetic constants used a fluorogenic peptide substrate, typically Boc-Val-Leu-Lys-AMC, and were determined in either 100 mM Mes/Tris/acetate, pH 7.0 containing 1 M NaCl and 10 mM 2-mercaptoethanol or 100 mM Na phosphate, pH 5.5 containing 1 M NaCl and 10 mM 2-mercaptoethanol. The enzyme concentration used was 2 nM. The stock substrate solution was prepared at 10 mM in DMSO. Screens were carried out at a fixed substrate concentration of 80 μM and detailed kinetic studies with doubling dilutions of substrate from 250 μM. The total DMSO concentration in the assay was kept below 3%. All assays were conducted at ambient temperature. Product fluorescence (excitation at 390 nm, emission at 460 nm) was monitored with a Labsystems Fluoroskan Ascent fluorescent plate reader. Product progress curves were generated over 15 minutes following generation of AMC product.

### Inhibition Studies

Potential inhibitors were screened using the above assay with variable concentrations of the compounds in the table below. These compounds were prepared on solid phase using the methodology outlined above. Reactions were initiated by addition of enzyme to buffered solutions of substrate and inhibitor. Kᵢ values were calculated according to equation 1

$${v}_{0} = \frac{VS}{{K}_{M} ( 1 + \frac{I}{{K}_{i}} ) + S} \qquad ( 1 )$$

where v₀ is the velocity of the reaction, V is the maximal velocity, S is the concentration of substrate with Michaelis constant of K_{M}, and I is the concentration of inhibitor.

The compounds depicted in the table below above showed Kᵢ values (at pH 7) between 0.5 μM and 2.7 μM and are thus useful in the prophylaxis or treatment of parasite infections or infestations, such as malaria.

Naphthalene-2-carboxylic-acid-[3-methyl-1S-(2R-methyl-4-oxo-tetrahydro-furan-3S-ylcarbamoyl)-butyl]-amide

Benzofuran-2-carboxylic-acid-[3-methyl-1S-(2R-methyl-4-oxo-tetrahydro-furan-3S-ylcarbamoyl)-butyl]-amide

5-Methoxy-benzofuran-2-carboxylic-acid-[3-methyl-1S-(2R-methyl-4-oxo-tetrahydro-furan-3S-ylcarbamoyl)-butyl]-amide

4-Acetylamino-N-[3-methyl-1S-(2R-methyl-4-oxo-tetrahydro-furan-3S-ylcarbamoyl)-butyl]-benzamide

4-Hydroxy-N-[3-methyl-1S-(2R-methyl-4-oxo-tetrahydro-furan-3S-ylcarbamoyl)-butyl]-3-morpholin-4-ylmethyl-benzamide

Biphenyl-4-carboxylic-acid-[3-methyl-1S-(2R-methyl-4-oxo-tetrahydro-furan-3S-ylcarbamoyl)-butyl]-amide

4-tert-Butyl-N-[3-methyl-1S-(2R-methyl-4-oxo-tetrahydro-furan-3S-ylcarbamoyl)-butyl]-benzamide

Benzothiazole-5-carboxylic-acid-[3-methyl-1S-(2R-methyl-4-oxo-tetrahydro-furan-3S-ylcarbamoyl)-butyl]-amide

1. A compound of the formula (IV): 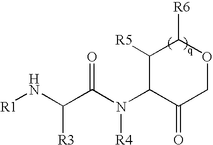
where:
R1=R′C(O),
R′=a phenyl substituted with at least one member of the group consisting of a pyrrolidinyl group, piperidinyl group, morpholinyl group and piperazinyl group and wherein said phenyl is optionally substituted with H, C1-7alkyl, C3-6cycloalkyl, OH, SH, NH₂, NHC1-3alkyl, N(C1-3alkyl)₂ or halogen
R4=H, C1-7-alkyl, Ar—C1-7-alkyl, Ar, C3-7-cycloalkyl; C2-7alkenyl\overline{,};
R3=C1-7-alkyl, C2-C7 alkenyl, C3-7-cycloalkyl, Ar—C1-7-alkyl, Ar;
R5=C1-7-alkyl, halogen, Ar—C1-7-alkyl, C0-3-alkyl-CONR3R4 or R^{iv};
R^{iv}═ 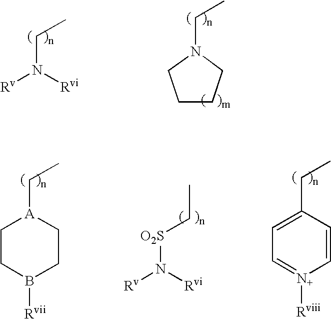
where n=1-3, m=1-3;
R^{v}, R^{vi}═H, C1-7-alkyl;
A=N, CH; B═N, O, S, CH;
R^{vii}=absent when B═O, S; or R^{vii}═H, C1-7-alkyl when B═N, CH;
R^{viii}═O, C1-7-alkyl;
R₆═H, C1-7-alkyl, Ar—C1-7-alkyl, C1-3-alkyl-SO2-R^{ix}, C1-3-alkyl-C(O)—NHR^{ix} or CH₂XAr,
R^{ix} is C1-7-alkyl. ArC1-7-alkyl or C3-C6-cycloaklyl;
q is 0 or 1;
wherein
each C1-3 alkyl or C1-7-alkyl (used alone or in composite expressions) is optionally substituted by one or two halogens and/or a heteroatom S, O, NH, in which a heteroatom located at a chain terminus is optionally substituted with one or 2 hydrogen atoms or an S heteroatom is optionally oxidised to the sulphone;
each C3-6 or C3-7 cycloalkyl comprises a C1-7-alkyl which additionally contains a C3-6 or C3-7 carbocyclic ring, respectively; or the C3-6 or C3-7 cycloalkyl is spiro bound to the adjacent carbon without an intervening C1-C7;
each Ar—C1-7-alkyl comprises a phenyl, pyrazolyl, pyridyl, imidazolyl, oxazolyl, isoxazolyl, thiazinolyl, isothiazinolyl, thiazolyl, oxadiazolyl, 1,2,3-triazolyl, 1,2,4-triazolyl, furanyl or thienyl aromatic ring (Ar) attached through a C1-7-alkyl, which aromatic ring Ar is optionally substituted with halogen, C1-3-alkyl, OH, OC1-3-alkyl, SH, SC1-3-alkyl or amine;
and pharmaceutically acceptable salts thereof.

2. A compound according to claim 1, wherein R4 and/or R6 is hydrogen.

3. A compound according to claim 1, wherein the R′ bicyclic ring is selected from naphthyl, quinolyl, benzofuranyl, benzothienyl, indolyl, indolinyl.

4. A compound according to claim 3, wherein the linkage is the 2 position of the R′ ring.

5. A compound according to claim 1, wherein R′ is substituted with morpholine or N-methylpiperidine linked through an alkyl or alkylether linkage.

6. A compound according to claim 1, wherein R1 is R′C(O).

7. A compound according to claim 1, wherein R3 is 2-methylprop-1-enyl, benzyl or especially i-butyl.

8. A compound according to claim 1, wherein the stereochemistry at R3 corresponds to a natural or non natural L-amino acid.

9. A compound according to claim 1, wherein R5 is CH₃, C₂H₅, CH₂Ar, CH₂CONH₂, (CH₂)₂CONH₂, CH₂OH

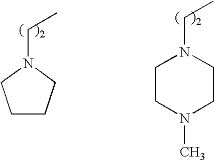

10. A compound according to claim 9, wherein R5 is CH₃, CH₂CH₃, or CH₂OH.

11. A compound according to claim 1, wherein R5 and the C4 bond both have (R) stereochemistry.

12. A compound according to claim 1, wherein R5 and the C4 bond both have (S) stereochemistry.

13. A compound according to claim 1, wherein q is 1.

14. A compound according to claim 1, wherein q is 0.

15. A compound according to claim 1, wherein R′ is a monocyclic ring substituted with a cyclic substituent.

16. A compound according to claim 15, wherein the monocyclic ring is pyridyl, pyrimidinyl or phenyl which is preferably substituted in the 3 or 4 position.

17. A compound according to claim 15, wherein the cyclic substituent is non-aromatic.

18. A compound according to claim 17, wherein the non-aromatic cyclic substituent is selected from the group consisting of pyrrolidine-1-yl, piperidine-1-yl, morpholin-4-yl, 4-methylpiperazin-1-yl, 2-morpholin-4-yl-ethylamino, and piperazin-1-yl.

19. A method for the treatment of disorders dependent upon the activity of cathepsin K comprising the administration of a compound as defined in claim 1 to a mammal in need thereof.

20. A method according to claim 19, wherein the disorder is a bone disorder such as periodontitis or osteoarthritis.

21. A method according to claim 19, wherein the disorder is a cartilage or matrix degradation disorder such as osteoarthritis or rheumatoid arthritis.

22. A method according to claim 19, wherein the disorder is a neoplasia.

23. A method for the treatment of a parasite infection comprising the administration of a compound as defined in claim 1 to a mammal in need thereof.

24. A method for the control of parasites comprising the administration of a compound as defined in claim 1 to an invertebrate vector and/or to a locus prone to infestation of such a vector.

25. A method for the preparation of a compound as defined in claim 1, comprising the steps of manipulating the protecting groups on a suitably protected carbohydrate derivative to effect deoxygenation at the anomeric postion, introducing the R5 substituent via a ketone functionality, for example by Wittig chemistry, introducing the 4-amino group by further manipulation of the C4 secondary alcoholn functionality to provide a protected 4-amino-5-substituted pyranol, N-extending the amine function using peptide chemistry and adding the R′C(═O) or R′S(═O)₂ capping group, further comprising the step of oxidising the pyranol before or after the N-terminal extension and/or capping.
